SEQUENCE LISTING (3 sequences)

SEQ ID NO: 1 - protein, 17 aa, Artificial Sequence
  Synthetic Polypeptide
KKKYPGGSTPVSSANMM

SEQ ID NO: 2 - DNA, 26 bp, Artificial Sequence
  Synthetic Polynucleotide
gctagcactagtatgagcgggtttaa

SEQ ID NO: 3 - DNA, 26 bp, Artificial Sequence
  Synthetic Polynucleotide
taatatgcggccgcttagtcaaaggt

(19) United States
(12) Patent Application Publication
(10) Pub. No.: US 2017/0166558 A1
(43) Pub. Date: Jun. 15, 2017

(54) O-GLCNAC TRANSFERASE (OGT) INHIBITORS AND USES THEREOF

(71) Applicants: President and Fellows of Harvard College, Cambridge, MA (US); The UNITED STATES OF AMERICA, as represented by the Secretary, Department of Health and Human, Bethesda, MD (US)

(72) Inventors: Suzanne Walker Kahne, Brookline, MA (US); Rodrigo Fermin Ortiz Meoz, Collegeville, PA (US); Craig Joseph Thomas, Gaithersburg, MD (US); Damien Yves Duveau, Germantown, MD (US)

(73) Assignees: President and Fellows of Harvard College, Cambridge, MA (US); The UNITED STATES OF AMERICA, as represented by the Secretary, Department of Health and Human, Bethesda, MD (US)

(21) Appl. No.: 15/323,206

(22) PCT Filed: Jul. 1, 2015
(86) PCT No.: PCT/US15/38792

§ 371 Date: Dec. 30, 2016

Related U.S. Application Data
(60) Provisional application No. 62/019,528, filed on Jul. 1, 2014.

(51) Int. Cl. C07D 409/14; C07D 401/12; C07D 405/12; C07D 409/12; C07D 215/227

(52) U.S. Cl. CPC C07D 409/14; C07D 409/12; C07D 215/227; C07D 405/12; C07D 401/12

(57) ABSTRACT

The present invention provides inhibitors of O-GlcNAc transferase. Typically, the inhibitors are quinolinone-6-sulfonamides. The invention also provides pharmaceutical compositions thereof and methods for using the same in diabetes and complications thereof, metabolic diseases, neurodegenerative diseases, proliferative diseases (e.g., cancers), autoimmune diseases, and inflammatory diseases.

### RELATED APPLICATION

The present application claims priority under 35 U.S.C. §119(e) to U.S. provisional patent application, U.S. Ser. No. 62/019,528, filed Jul. 1, 2014, which is incorporated herein by reference.

### FIELD OF INVENTION

The present application relates to OGT inhibitors. The present invention also provides compositions of the OGT inhibitors and methods of treating OGT-associated diseases and conditions.

### BACKGROUND OF THE INVENTION

The hexosamine biosynthetic pathway (HSP) is a minor branch of the glycolytic pathway, diverting 3-5% of cellular glucose toward the synthesis of UDP-GlcNAc, which is either transported to the Golgi and used in the synthesis of complex glycans or remains in the cytoplasm where it is the substrate for O-GlcNAc transferase (OGT). OGT is the sole known enzyme to catalyze the glycosylation of serine and threonine residues on many nuclear and cytoplasmic proteins (termed O-GlcNAcylation). This post-translational modification is dynamic and is a general mechanism, like protein phosphorylation, of signal transduction.

Excess flux through the HSP has been implicated in both the early (insulin resistance) and late (nephropathy, microvascular damage) stages of diabetes mellitus, both in vivo and in vitro. Diabetes involves a deficiency in the availability and/or utilization of insulin. Insulin is a hormone produced by the pancreas and is necessary for cells to utilize glucose. Insulin resistance is a condition in which muscle, fat, and liver cells do not use insulin properly. As a result, the pancreas produces more insulin, which is also not used properly. Eventually, the pancreas cannot keep up with the body's need for insulin, and excess glucose builds up in the bloodstream. Thus, in insulin resistance, there may be high levels of blood glucose and high levels of insulin circulating in the bloodstream at the same time.

Experiments have shown that insulin resistance due to increased hexosamine flux is caused by hyper O-GlcNAcylation. Diabetics have increased production of two adipokines directly responsible for vascular injury, plasminogen activator inhbitor-1 (PAI-1) and transforming growth factor β1 (TGF-β1). Transcription of both of these proteins is decreased in cell culture when levels of O-GlcNAcylation are decreased. The molecular mechanism for this is known; increased transcription is mediated by the O-GlcNAcylation state of the transcription factor Sp1.

OGT activity and O-GlcNAcylation have also been implicated in other disease states, such as neurodegenerative diseases, cancer, autoimmune diseases, and inflammatory diseases. Accordingly, there is a need to find OGT inhibitors useful as therapeutic agents.

### SUMMARY OF THE INVENTION

The invention relates in part to compounds that inhibit O-GlcNAc transferase (OGT) activity. Compounds of the invention inhibit O-GlcNAcylation by OGT. O-GlcNAcylation is the glycosylation of serine and/or threonine residues on nuclear and cytoplasmic proteins that is catalyzed by OGT. Compounds of the invention are useful for the treatment of diseases, disorders, and conditions associated with hyper or aberrant O-GlcNAcylation (e.g., diabetes and complications thereof, cancers, neurodegenerative diseases, autoimmune diseases, and inflammatory diseases).

In one aspect, inventive compounds are generally of Formula (I):

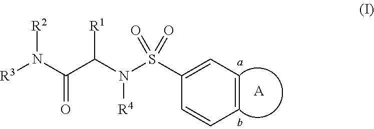

or a pharmaceutically acceptable salt thereof,

wherein

Ring A is of the formula

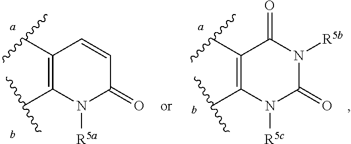

wherein a and b indicate the points of attachment to the phenyl ring;

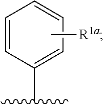

R¹ is n-butyl, thiophene, —CH₂-Ph, cyclohexyl, or of the formula:

R^{1a} is hydrogen, halogen, —OR⁰, or optionally substituted C₁₋₄ alkyl;

R⁰ is hydrogen or C₁₋₄ alkyl; each of R² and R³ is independently hydrogen, optionally substituted C₁₋₄ alkyl, optionally substituted thiophenyl-C₁₋₄alkylene, or optionally substituted furanyl-C₁₋₄ alkylene;

R⁴ is hydrogen, optionally substituted C₁₋₆ alkyl, or a nitrogen protecting group;

each of R^{5a}, R^{5b}, and R^{5c} is independently hydrogen, optionally substituted C₁₋₆ alkyl, or a nitrogen protecting group;

R¹ and R⁴ may optionally be taken together with the intervening nitrogen to form an optionally substituted heteroaryl or optionally substituted heterocycle;

R² and R³ may optionally be taken together with the intervening nitrogen to form an optionally substituted six-membered heterocycle.

In another aspect, the present invention provides methods of treatment comprising administering an inventive compound to a subject. The compounds of the invention or pharmaceutical compositions thereof may be used to treat any disease including diabetes and complications thereof, insulin resistance, neurodegenerative diseases such as Alzheimer's disease, cancer, autoimmune diseases, and inflammatory diseases. The compounds of the invention may be used to treat disease in humans and other animals including domesticated and experimental animals. The inventive compounds may also be used as probes of biological pathways.

In yet another aspect, the present invention provides pharmaceutical compositions comprising the inventive compounds. The composition typically comprises a therapeutically effective amount of an inventive compound to inhibit OGT and/or treat diabetes and complications thereof, insulin resistance, neurodegenerative diseases such as Alzheimer's disease, cancer, autoimmune diseases, and inflammatory diseases. The pharmaceutical compositions may optionally include a pharmaceutically acceptable excipient. Any mode of administration including oral, parenteral, and topical administration of the inventive compound or pharmaceutical composition thereof may be used.

In another aspect, the present invention provides kits comprising a compound of Formula (I), or a pharmaceutically acceptable salt, solvate, hydrate, polymorph, co-crystal, tautomer, stereoisomer, isotopically labeled derivative, or prodrug thereof, or a pharmaceutical composition thereof. The kits of the invention may include a single dose or multiple doses of a compound of Formula (I), or a pharmaceutically acceptable salt, solvate, hydrate, polymorph, co-crystal, tautomer, stereoisomer, isotopically labeled derivative, or prodrug thereof, or a pharmaceutical composition thereof. The provided kits may be useful for the treatment of proliferative diseases, inflammatory diseases, autoimmune diseases, autoinflammatory diseases, and metabolic diseases. In certain embodiments, the kits described herein further include instructions for administering the compound of Formula (I), or the pharmaceutically acceptable salt, solvate, hydrate, polymorph, co-crystal, tautomer, stereoisomer, isotopically labeled derivative, or prodrug thereof, or the pharmaceutical composition thereof. The kits may also include packaging information describing the use or prescribing information for the subject or a health care professional. Such information may be required by a regulatory agency such as the U.S. Food and Drug Administration (FDA). The kit may also optionally include a device for administration of the compound or composition, for example, a syringe for parenteral administration.

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987. The disclosure is not intended to be limited in any manner by the exemplary listing of substituents described herein.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, Ind. 1972). The disclosure additionally encompasses compounds described herein as individual isomers substantially free of other isomers, and alternatively, as mixtures of various isomers.

When a range of values is listed, it is intended to encompass each value and sub-range within the range. For example “C₁₋₆” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆.

The term “aliphatic” includes both saturated and unsaturated, straight chain (i.e., unbranched), branched, acyclic, cyclic, or polycyclic aliphatic hydrocarbons, which are substituted or unsubstituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, the term “alkyl” includes straight, branched and cyclic alkyl groups. An analogous convention applies to other generic terms such as “alkenyl”, “alkynyl”, and the like. Furthermore, the terms “alkyl”, “alkenyl”, “alkynyl”, and the like encompass both substituted and unsubstituted groups. In certain embodiments, “lower alkyl” is used to indicate those alkyl groups (cyclic, acyclic, substituted, unsubstituted, branched or unbranched) having 1-6 carbon atoms.

In certain embodiments, the alkyl, alkenyl, and alkynyl groups employed in the disclosure contain 1-20 aliphatic carbon atoms. In certain other embodiments, the alkyl, alkenyl, and alkynyl groups employed in the disclosure contain 1-10 aliphatic carbon atoms. In yet other embodiments, the alkyl, alkenyl, and alkynyl groups employed in the disclosure contain 1-8 aliphatic carbon atoms. In still other embodiments, the alkyl, alkenyl, and alkynyl groups employed in the disclosure contain 1-6 aliphatic carbon atoms. In yet other embodiments, the alkyl, alkenyl, and alkynyl groups employed in the disclosure contain 1-4 carbon atoms. Illustrative aliphatic groups thus include, but are not limited to, for example, methyl, ethyl, n-propyl, isopropyl, cyclopropyl, —CH₂-cyclopropyl, vinyl, allyl, n-butyl, sec-butyl, isobutyl, tert-butyl, cyclobutyl, —CH₂-cyclobutyl, n-pentyl, sec-pentyl, isopentyl, tert-pentyl, cyclopentyl, —CH₂-cyclopentyl, n-hexyl, sec-hexyl, cyclohexyl, —CH₂-cyclohexyl moieties and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl (propargyl), 1-propynyl, and the like.

The term “alkyl” refers to a radical of a straight-chain or branched saturated hydrocarbon group having from 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl). Additional examples of alkyl groups include n-heptyl (C₇), n-octyl (C₈), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₆ alkyl, e.g., —CH₃). In certain embodiments, the alkyl group is a substituted C₁₋₁₀ alkyl (such as substituted C₁₋₆ alkyl, e.g., —CF₃).

“Alkenyl” refers to a radical of a straight-chain or branched hydrocarbon group having from 2 to 20 carbon atoms, one or more carbon-carbon double bonds, and no triple bonds (“C₂₋₂₀ alkenyl”). In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, i.e., unsubstituted (an “unsubstituted alkenyl”) or substituted (a “substituted alkenyl”) with one or more substituents. In certain embodiments, the alkenyl group is unsubstituted C₂₋₁₀ alkenyl. In certain embodiments, the alkenyl group is substituted C₂₋₁₀ alkenyl. In an alkenyl group, a C═C double bond for which the stereochemistry is not specified (e.g., —CH═CHCH₃ or

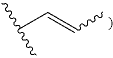

may be an (E)- or (Z)-double bond.

“Alkynyl” refers to a radical of a straight-chain or branched hydrocarbon group having from 2 to 20 carbon atoms, one or more carbon-carbon triple bonds, and optionally one or more double bonds (“C₂₋₂₀ alkynyl”). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently optionally substituted, i.e., unsubstituted (an “unsubstituted alkynyl”) or substituted (a “substituted alkynyl”) with one or more substituents. In certain embodiments, the alkynyl group is unsubstituted C₂₋₁₀ alkynyl. In certain embodiments, the alkynyl group is substituted C₂₋₁₀ alkynyl.

“Carbocyclyl” or “carbocyclic” refers to a radical of a non-aromatic cyclic hydrocarbon group having from 3 to 10 ring carbon atoms (“C₃₋₁₀ carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclic ring, as defined above, is fused with one or more aryl or heteroaryl groups wherein the point of attachment is on the carbocyclic ring, and in such instances, the number of carbons continue to designate the number of carbons in the carbocyclic ring system. Unless otherwise specified, each instance of a carbocyclyl group is independently optionally substituted, i.e., unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is substituted C₃₋₁₀ carbocyclyl.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl.

“Heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged, or spiro ring system, such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclic ring, as defined above, is fused with one or more carbocyclyl groups wherein the point of attachment is either on the carbocyclyl or heterocyclic ring, or ring systems wherein the heterocyclic ring, as defined above, is fused with one or more aryl or heteroaryl groups, wherein the point of attachment is on the heterocyclic ring, and in such instances, the number of ring members continue to designate the number of ring members in the heterocyclic ring system. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered, non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiiranyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, and dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Aryl” refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups, wherein the radical or point of attachment is on the aryl ring, and in such instances, the number of carbon atoms continue to designate the number of carbon atoms in the aryl ring system. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

“Aralkyl” refers to a substituted or unsubstituted alkyl group substituted by a substituted or unsubstituted aryl group. In certain embodiments, the aralkyl is substituted or unsubstituted benzyl. In certain embodiments, the aralkyl is benzyl. In certain embodiments, the aralkyl is substituted or unsubstituted phenethyl. In certain embodiments, the aralkyl is phenethyl.

“Heteroaryl” refers to a radical of a 5-10 membered, monocyclic or bicyclic 4n+2 aromatic ring system (e.g., having 6 or 10 pi electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5-14 membered heteroaryl. In certain embodiments, the heteroaryl group is substituted 5-14 membered heteroaryl.

Exemplary 5-membered heteroaryl groups containing one heteroatom include, without limitation, pyrrolyl, furanyl, and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

“Heteroaralkyl” is a subset of alkyl and heteroaryl and refers to a substituted or unsubstituted alkyl group substituted by a substituted or unsubstituted heteroaryl group.

“Unsaturated” or “partially unsaturated” refers to a group that includes at least one double or triple bond. A “partially unsaturated” ring system is further intended to encompass rings having multiple sites of unsaturation, but is not intended to include aromatic groups (e.g., aryl or heteroaryl groups). Likewise, “saturated” refers to a group that does not contain a double or triple bond, i.e., contains all single bonds.

Alkyl, alkenyl, alkynyl, carbocyclyl, heterocyclyl, aryl, and heteroaryl groups, which are divalent linking groups, are further referred to using the suffix -ene, e.g., alkylene, alkenylene, alkynylene, carbocyclylene, heterocyclylene, arylene, and heteroarylene.

An atom, moiety, or group described herein may be unsubstituted or substituted, as valency permits, unless otherwise provided expressly. The term “optionally substituted” refers to substituted or unsubstituted.

A group is substituted or unsubstituted unless expressly provided otherwise. The term “optionally substituted” refers to being substituted or unsubstituted. In certain embodiments, alkyl, alkenyl, alkynyl, carbocyclyl, heterocyclyl, aryl, and heteroaryl groups are substituted or unsubstituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety. In certain embodiments, the substituent is a carbon atom substituent. In certain embodiments, the substituent is a nitrogen atom substituent. In certain embodiments, the substituent is an oxygen atom substituent. In certain embodiments, the substituent is a sulfur atom substituent.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb)}₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc}; each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;

each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;

each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;

each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl and 5-10 membered heteroaryl, or two R^{if} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and

each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C_{1-6 alkyl})₃, —OSi(C₁₋₆ alkyl)₃ —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, 5-10 membered heteroaryl; or two geminal Rgg substituents can be joined to form ═O or ═S; wherein X⁻ is a counterion.

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS (═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb}))₂, wherein R^{aa}, R^{bb}, and R^{cc} are as defined herein.

A “counterion” or “anionic counterion” is a negatively charged group associated with a cationic quaternary amino group in order to maintain electronic neutrality. Exemplary counterions include halide ions (e.g., F⁻, Cl⁻, Br⁻, I⁻), NO₃⁻, ClO₄⁻, OH⁻, H₂PO₄⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

“Halo” or “halogen” refers to fluorine (fluoro, —F), chlorine (chloro, —Cl), bromine (bromo, —Br), or iodine (iodo, —I).

“Acyl” refers to a moiety selected from the group consisting of —C(═O)R^{aa}, —CHO, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —C(═O)NR^{bb}SO₂R^{aa}, —C(═S)N(R^{bb})₂, —C(═O)SR^{aa}, or —C(═S)SR^{aa}, wherein R^{aa} and R^{bb} are as defined herein.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂0R^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on a nitrogen atom is a nitrogen protecting group (also referred to as an amino protecting group). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitrophenylacetamide, o-nitrophenoxyacetamide, acetoacetamide, (N-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, and o-(benzoyloxymethyl)benzamide.

Nitrogen protecting groups such as carbamate groups (e.g., —C(═O)OR^{aa}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluorenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(ptoluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl] amine (MMTr), N-9-phenylfluorenylamine (PhF), N-2,7-dichloro-9-fluorenylmethyleneamine, N-ferrocenylmethylamino (Fcm), N-2-picolylamino oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen atom substituents include, but are not limited to, —R^{aa}, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{cc} are as defined herein. In certain embodiments, the oxygen atom substituent present on an oxygen atom is an oxygen protecting group (also referred to as a hydroxyl protecting group). Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference. Exemplary oxygen protecting groups include, but are not limited to, methyl, t-butyloxycarbonyl (BOC or Boc), methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on an sulfur atom is a sulfur protecting group (also referred to as a “thiol protecting group”). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)_{2,} wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

A “hydrocarbon chain” refers to a substituted or unsubstituted divalent alkyl, alkenyl, or alkynyl group. A hydrocarbon chain includes (1) one or more chains of carbon atoms immediately between the two radicals of the hydrocarbon chain; (2) optionally one or more hydrogen atoms on the chain(s) of carbon atoms; and (3) optionally one or more substituents (“non-chain substituents,” which are not hydrogen) on the chain(s) of carbon atoms. A chain of carbon atoms consists of consecutively connected carbon atoms (“chain atoms” or “carbon units”) and does not include hydrogen atoms or heteroatoms. However, a non-chain substituent of a hydrocarbon chain may include any atoms, including hydrogen atoms, carbon atoms, and heteroatoms. For example, hydrocarbon chain —C^{A}H(C^{B}H₂C^{C}H₃)— includes one chain atom C^{A}, one hydrogen atom on C^{A}, and non-chain substituent —(C^{B}H₂C^{C}H₃). The term “Cₓ hydrocarbon chain,” wherein x is a positive integer, refers to a hydrocarbon chain that includes x number of chain atom(s) between the two radicals of the hydrocarbon chain. If there is more than one possible value of x, the smallest possible value of x is used for the definition of the hydrocarbon chain. For example, —CH(C₂H₅)— is a C₁ hydrocarbon chain, and

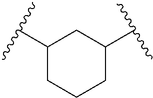 [SMILES: *C1CCCC(*)C1]

is a C₃ hydrocarbon chain. When a range of values is used, the meaning of the range is as described herein. For example, a C₃₋₁₀ hydrocarbon chain refers to a hydrocarbon chain where the number of chain atoms of the shortest chain of carbon atoms immediately between the two radicals of the hydrocarbon chain is 3, 4, 5, 6, 7, 8, 9, or 10. A hydrocarbon chain may be saturated (e.g., —(CH₂)₄—). A hydrocarbon chain may also be unsaturated and include one or more C═C and/or C≡C bonds anywhere in the hydrocarbon chain. For instance, —CH═CH—(CH₂)₂—, —CH₂—C≡C—CH₂—, and —C≡C—CH═CH— are all examples of a unsubstituted and unsaturated hydrocarbon chain. In certain embodiments, the hydrocarbon chain is unsubstituted (e.g., —C≡C— or —(CH₂)₄—). In certain embodiments, the hydrocarbon chain is substituted (e.g., —CH(C₂H₅)— and —CF₂—). Any two substituents on the hydrocarbon chain may be joined to form a substituted or unsubstituted carbocyclyl, substituted or unsubstituted heterocyclyl, substituted or unsubstituted aryl, or substituted or unsubstituted heteroaryl ring. For instance,

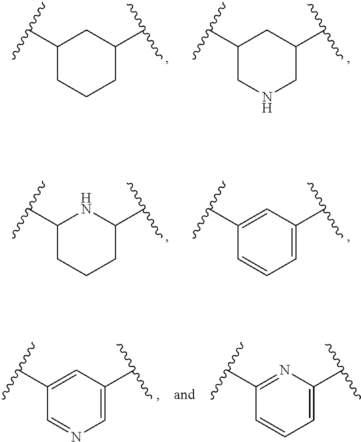 [SMILES: *C1CCCC(*)C1 ; *C1CNCC(*)C1 ; *C1CCCC(*)N1 ; *c1cccc(*)c1 ; *c1cncc(*)c1 ; *c1cccc(*)n1]

are all examples of a hydrocarbon chain. In contrast, in certain embodiments,

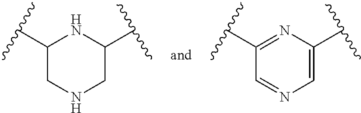 [SMILES: *C1CNCC(*)N1 ; *c1cncc(*)n1]

are not within the scope of the hydrocarbon chains described herein. When a chain atom of a Cₓ hydrocarbon chain is replaced with a heteroatom, the resulting group is referred to as a Cₓ hydrocarbon chain wherein a chain atom is replaced with a heteroatom, as opposed to a Cₓ₋₁ hydrocarbon chain. For example,

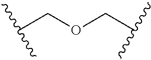 [SMILES: *COC*]

is a C₃ hydrocarbon chain wherein one chain atom is replaced with an oxygen atom.

The term “leaving group” is given its ordinary meaning in the art of synthetic organic chemistry and refers to an atom or a group capable of being displaced by a nucleophile. Examples of suitable leaving groups include, but are not limited to, halogen (such as F, Cl, Br, or I (iodine)), alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates. In some cases, the leaving group is a sulfonic acid ester, such as toluenesulfonate (tosylate, —OTs), methanesulfonate (mesylate, —OMs), p-bromobenzenesulfonyloxy (brosylate, —OBs), —OS(═O)₂(CF₂)₃CF₃ (nonaflate, —ONf), or trifluoromethanesulfonate (triflate, —OTf). In some cases, the leaving group is a brosylate, such as p-bromobenzenesulfonyloxy. In some cases, the leaving group is a nosylate, such as 2-nitrobenzenesulfonyloxy.The leaving group may also be a phosphineoxide (e.g., formed during a Mitsunobu reaction) or an internal leaving group such as an epoxide or cyclic sulfate. Other non-limiting examples of leaving groups are water, ammonia, alcohols, ether moieties, thioether moieties, zinc halides, magnesium moieties, diazonium salts, and copper moieties.

### Other Definitions

The following definitions are more general terms used throughout the present application.

The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The terms “composition” and “formulation” are used interchangeably.

A “subject” to which administration is contemplated includes, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., cynomolgus monkeys, rhesus monkeys); commercially relevant mammals such as cattle, pigs, horses, sheep, goats, cats, and/or dogs) and birds (e.g., commercially relevant birds such as chickens, ducks, geese, and/or turkeys). In certain embodiments, the animal is a mammal. The animal may be a male or female at any stage of development. The animal may be a transgenic animal or genetically engineered animal. In certain embodiments, the subject is a non-human animal. In certain embodiments, the animal is a fish or reptile. A “patient” refers to a human subject in need of treatment of a disease. The subject may also be a plant. In certain embodiments, the plant is a land plant. In certain embodiments, the plant is a non-vascular land plant. In certain embodiments, the plant is a vascular land plant. In certain embodiments, the plant is a seed plant. In certain embodiments, the plant is a cultivated plant. In certain embodiments, the plant is a dicot. In certain embodiments, the plant is a monocot. In certain embodiments, the plant is a flowering plant. In some embodiments, the plant is a cereal plant, e.g., maize, corn, wheat, rice, oat, barley, rye, or millet. In some embodiments, the plant is a legume, e.g., a bean plant, e.g., soybean plant. In some embodiments, the plant is a tree or shrub.

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The terms “condition,” “disease,” and “disorder” are used interchangeably.

An “effective amount” of a compound described herein refers to an amount sufficient to elicit the desired biological response, i.e., treating the condition. As will be appreciated by those of ordinary skill in this art, the effective amount of a compound described herein may vary depending on such factors as the desired biological endpoint, the pharmacokinetics of the compound, the condition being treated, the mode of administration, and the age and health of the subject. An effective amount encompasses therapeutic and prophylactic treatment.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent.

A “prophylactically effective amount” of a compound described herein is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

As used herein, the terms “O-GlcNAcylation-associated disease or disorder” and “OGT-associated disease or disorder” include, but are not limited to diseases and disorders in which there is abnormal OGT activity and/or abnormal levels of O-GlcNAcylation. As used herein, the term “OGT activity” means OGT-mediated O-GlcNAcylation. An abnormal level of OGT activity and/or O-GlcNAcylation may be a level that is higher than a normal level or may be a level that is lower than a normal level, wherein a “normal” level is the level in a subject who does not have a disease or disorder associated with OGT activity or O-GlcNAcylation. Examples of diseases and disorders associated with OGT activity and/or O-GlcNAcylation levels include, but are not limited to neurodegenerative disorders such as Alzheimer's disease; cancer; metabolic diseases such as diabetes mellitus, insulin resistance, and complications of diabetes; and other OGT-associated diseases.

As used herein, the term “neurodegenerative disorders” refer to a type of neurological disease marked by the loss of nerve cells, including, but not limited to, Alzheimer's disease, Parkinson's disease, amyotrophic lateral sclerosis, tauopathies (including frontotemporal dementia), and Huntington's disease.

As used herein, the term “metabolic disease” refers to any disease or disorder that involves an alteration in the normal metabolism of carbohydrates, lipids, proteins, nucleic acids, or a combination thereof. A metabolic disease is associated with either a deficiency or excess in a metabolic pathway resulting in an imbalance in metabolism of nucleic acids, proteins, lipids, and/or carbohydrates. Factors affecting metabolism include, and are not limited to, the endocrine (hormonal) control system (e.g., the insulin pathway, the enteroendocrine hormones including GLP-1, PYY or the like), the neural control system (e.g., GLP-1 in the brain), or the like. Examples of metabolic diseases include, but are not limited to, diabetes (e.g., type 1 diabetes, type 2 diabetes, gestational diabetes), hyperglycemia, hyperinsulinemia, insulin resistance, and obesity.

As used herein, the term “complication of diabetes” is used to mean a disorder that is associated with diabetes. Non-limiting examples of complications of diabetes include microvascular damage, insulin resistance, vascular damage, nephropathy, skin ulcers, circulatory damage, diabetic nephropathy, diabetic retinopathy, macro-vascular disease, micro-vascular disease, cardiac dysfunction, and diabetic neuropathy.

The term “diabetic” as used herein, means a subject who, at the time the sample is taken, has a primary deficiency of insulin. The term diabetic includes, but is not limited to, individuals with juvenile diabetes (Type 1 diabetes), adult-onset diabetes (Type 2 diabetes), gestational diabetes, and any other conditions of insulin deficiency. The terms “diabetic” and “diabetes” are terms of art, known and understood by those practicing in the medical profession, a formal definition of which can be found in Harrison's Principles of Medicine (Harrisons, Vol 14, Principles of Internal Medicine, Eds. Fauci, A. S., E. Braunwald, K. J. Isselbacher, J. D. Wilson, J. B. Martin, D. L. Kasper, S. L. Hauser, D. L. Longo, McGraw-Hill, New York, 1999).

Subjects with blood glucose levels that are higher than normal but not yet in the range associated with a diagnosis of diabetes may be considered to have “pre-diabetes.” Pre-diabetes is also known in the art as “impaired fasting glucose” (IFG) or “impaired glucose tolerance” (IGT). Subjects with pre-diabetes have a higher risk of developing type 2 diabetes, which is also known as adult-onset diabetes or noninsulin-dependent diabetes.

“Insulin resistance,” as used herein, is a condition in which the tissues of the body fail to respond normally to insulin. DeFronzo, R. A. J. Cardiomuscular Pharmacology 20 (Suppl. 11): S1-S16 (1992). Insulin resistance manifests itself in pathologically elevated endogenous insulin and glucose levels and predisposes one who suffers from said resistance to the development of a cluster of abnormalities, including some degree of impaired glucose tolerance, an increase in plasma triglycerides and low density lipoprotein cholesterol (LDL) levels, a decrease in high-density lipoprotein cholesterol (HDL) levels, high blood pressure, hyperuricemia, a decrease in plasma fibrinolytic activity, an increase in cardiovascular disease and atherosclerosis. Reaven, G. M. Physiol-Rev. 75(3): 473-86 (1995).

“Cancer” as used herein refers to an uncontrolled growth of cells which interferes with the normal functioning of the bodily organs and systems. Cancers which migrate from their original location and seed vital organs can eventually lead to the death of the subject through the functional deterioration of the affected organs. Carcinomas are malignant cancers that arise from epithelial cells and include adenocarcinoma and squamous cell carcinoma. Sarcomas are cancer of the connective or supportive tissue and include osteosarcoma, chondrosarcoma and gastrointestinal stromal tumor. Hematopoietic cancers, such as leukemia, are able to outcompete the normal hematopoietic compartments in a subject, thereby leading to hematopoietic failure (in the form of anemia, thrombocytopenia and neutropenia) ultimately causing death. A person of ordinary skill in the art can classify a cancer as a sarcoma, carcinoma or hematopoietic cancer.

### BRIEF DESCRIPTION OF THE DRAWINGS

FIG. 1 shows that OGT catalyzes the addition of GlcNAc to proteins. FIG. 1A shows that OGT and OGA are responsible for the dynamic modification of proteins with GlcNAc. OGT adds O-GlcNAc to proteins and OGA removes this modification. FIG. 1B shows that histone H2B can be glycosylated by OGT, Glycosylation of c-Myc by OGT blocks its degradation in certain cell types.

FIG. 2 shows a fluorescent displacement screen for the discovery of an OGT inhibitor OSMI-1. FIG. 2A shows that initial screen uncovers Q6S scaffold and further study yields OGT inhibitor OSMI-1 (compound 1). FIG. 2B shows that OSMI-1 inhibits OGT glycosylation in vitro more efficiently than UDP-5S-GlcNAc. OSMI-1 is slightly more potent in vitro than than UDP-5S-GlcNAc (IC₅₀ values =3 μM+/−0.9 and 11+/−5.6 for OSMI-1 and UDP-5S-GlcNAc, respectively). FIG. 2C shows kinetic analysis of OSMI-1. OSMI-1 inhibits OGT in a competitive manner. Inhibition constant (Ki) was found to be 0.4+/−0.1 μM, approximately ten-fold below that for UDP-5S-GlcNAc (5+/−1 uM).

FIG. 3 shows that OSMI-1 can effectively inhibit OGT in mammalian cells. FIG. 3A shows immunoblotting of global O-GlcNAcylation after treatment of Chinese hamster ovary (CHO) cells with OSMI-1 and 4Ac-5SGlcNAc. CHO cells were previously used to evaluate 4Ac-5S-GlcNAc as an inhibitor. Cells were treated with concentrations of OSMI-1 and 4Ac-5S-GlcNAc ranging from 10-100 μM and, after 24 hr, cell lysates were separated by SDS-PAGE and immunoblotted with the O-GlcNAc antibody RL2. OSMI-1 reduced global O-GlcNAcylation in a dose-dependent manner (see FIG. 7), with the maximal reduction achieved at 50 μM. At this concentration, 4Ac-5S-GlcNAc appears to reduce global O-GlcNAcylation slightly more than OSMI-1. An 8 hr time course study performed with both compounds at 50 μM showed that global O-GlcNAcylation decreased more quickly with OSMI-1, most likely because 4Ac-5S-GlcNAcmust be metabolized to an active compound before it can inhibit OGT (see FIG. 8). Several other mammalian cell lines were examined, and it was found that OSMI-1 treatment substantially decreased global O-GlcNAcylation in all of them (see FIG. 9). FIG. 3B shows that biomarkers of OGT inhibition include a mass shift of Nup62 and a decrease in OGA levels. Because Nup62 is heavily O-GlcNAcylated, it shows a large mass shift (>2.5 Kd) when all ten O-GlcNAc residues are absent. Cellular Nup62 shifts to lower molecular weight following treatment of cells with either OSMI-1 or 4Ac-5S-GlcNAc, consistent with inhibition of OGT. Treatment of CHO cells with either OSMI-1 or 4Ac-5S-GlcNAc resulted in accumulation of sOGT, a short isoform of OGT while levels of full-length ncOGT remained unchanged. Concomitantly, OGA levels decreased under both conditions, most likely due to a feedback mechanism. FIG. 3C shows that OSMI-1 can decrease glycosylation of H2B-Ser112 in CHO cells. Effects of OSMI-1 on O-GlcNAcylation of histone H2B-S112 are evaluated. Histone H2B-S112 is one of the few protein-specific O-GlcNAc modifications for which an antibody is available (abcam). While total H2B levels remained unchanged, the O-GlcNAc modification was greatly reduced in OSMI-1-treated CHO cells. FIG. 3D shows that Lectins ConA, LCA and jacalin (JAC) can recognize extracellular glycans, which should not be affected by a specific inhibitor of OGT. Immunoblot of lectin ConA (left), LCA (middle) and JAC (right) show that OSMI-1 does not affect the composition of extracellular glycans. Lectins have been previously used to asses the composition of cell surface glycans. Ten different biotinylated lectins that recognize different features of N- and O-glycans (ConA, LCA, Jacalin, Pha-E, ECL, Pha-L, VVL, GSL-I, PNA and DBA) were used to visualize cell surface glycoproteins following incubation of CHO cells with either 50 μM OSMI-1 or 4Ac-5S-GlcNAc. Minimal changes were observed when ConA, PHA-L, ECL, VVL, GSL-I, PNA, or DBA was used indicating that the composition of the carbohydrate moieties recognized by these particular lectins is unaffected by OSMI-1 or 4Ac-5S-GlcNAc (see FIG. 12). For Jacalin, PHA-E and LCA, substantial changes were observed in blots of lysates from cells treated with 4Ac-5SGlcNAc, but OSMI-1-treated lysates were similar to the DMSO control. Jacalin detects the GalNAc-peptide portion of mucin-type O-glycans, and 4Ac-5SGlcNAc treatment resulted in mass shifts for several bands. These mass shifts suggest that some of the twenty enzymes that use UDP-GalNAc to initiate mucin-type O-glycan synthesis were inhibited by UDP-5SGalNAc, another product of the metabolism of 4Ac-5SGlcNAc. 4Ac-5SGlcNAc treatment also results in loss of signal intensity for the lectins PHA-E (see FIG. 12) and LCA, which recognizes core-fucosylated N-Glycans.

FIG. 4 shows that OGT inhibition decreases cell proliferation and c-Myc stability in LNCaP cells. Effects of OSMI-1 are evaluated on a human cancer cell line. OGT is upregulated in metastatic prostate cancer and high O-GlcNAcylation levels are correlated with a poor clinical prognosis. LNCaP cells, derived from a castration resistant metastatic prostate tumor, depend on abundant c-myc expression for rapid growth. Because it was previously shown that OGT knockdown reduces c-Myc abundance by destabilizing the protein, LNCaP cells were treated with 50 μM OSMI-1 for 6 and 48 hr. FIG. 4A shows cell viability of LNCaP cells treated for 48 hrs with 50 μM of indicated compounds. Treatment of LNCaP cells with OSMI-1 leads to a decrease in cell viability. FIG. 4B shows that treatment of LNCaP cells with OSMI-1 decreases c-Myc stability 6 and 48 hours post-treatment.

FIG. 5 shows in vitro study of both OSMI-1 and UDP-SS-GlcNAc. FIG. 5A shows Michaelis-Menten plot of OSMI-1. FIG. 5B shows Michaelis-Menten plot of UDP-SS-GlcNAc. FIG. 5C shows Vₘₐₓ and Kₘ values from Michaelis-Menten plot. FIG. 5D shows Kᵢ and standard error values of OSMI-1 and UDP-SS-GlcNAc from non-linear regression analysis fit competitive inhibition.

FIG. 6 shows computational modeling of OSMI-1 using the IFD protocol. Analysis of the docked model revealed similar interactions of F868, V895, H901 and R904 with the beta lactam moiety of OSMI-1 and uracil of UDP-SS-GlcNAc.

FIG. 7 shows that OSMI-1 inhibits global O-GlcNAcylation in a dose-dependent manner. CHO cells were grown to 50-60% confluency and treated with OGT inhibitors Ac4-5S-GlcNAc (left) or OSMI-1 (right) at the indicated concentrations for 24 hours. After treatment, cells were harvested and cell lysates were immunoblotted with the indicated antibodies. Both Ac4-5S-GlcNAc and OSMI-1 are able to reduce global O-GlcNAcylation in a dose-dependent manner. In the case of OSMI-1, maximum reduction is achieved when cells are treated with 50 μM OSMI-1; there was not observable benefit to treatment with higher concentrations due to the limited aqueous solubility of OSMI-1.

FIG. 8 shows that OSMI-1 reduces global O-GlcNAcylation rapidly in cells. CHO cells were grown to 50-60% confluency and treated with OGT inhibitors Ac-5S-GlcNAc (left) or OSMI-1 (right), at 50 μM for the indicated amount of time. After treatment, cells were harvested and cell lysates were immunoblotted with the indicated antibodies. Both Ac4-5S-GlcNAc and OSMI-1 reduce global O-GlcNAcylation levels after 8 hours; however, only OSMI-1, which does not require metabolic processing to become active, is able to reduce O-GlcNAc levels after just two hours.

FIG. 9 shows that OSMI-1 can inhibit O-GlcNAcylation in various mammalian cell types. FIG. 9A shows that OSMI-1 reduces O-GlcNAcylation in several mammalian cell types. CHO, HEK-293 HeLa and D283-Med cells (left to right) grown to 50-60% confluency were treated with either DMSO vehicle alone or 50 μM OGT inhibitor, Ac4-5SGlcNAc or OSMI-1, for 24 hours. After treatment, cells were harvested and cell lysates were immunoblotted with the indicated antibodies. Both Ac4-5SGlcNAc and OSMI-1 reduce global O-GlcNAc levels in all four lines examined. FIG. 9B shows that PC3-ML cells were grown at 70% confluency and treated with DMSO, 25 uM or 50 uM OGT inhibitor OSMI-1 for indicated time points. After treatment, cells were harvested and cell lysates were immunoblotted with the indicated antibodies. The results show that both Ac-SS-GlcNAc and OSMI-1 are able to reduce global O-GlcNAcylation levels at 24 hours in all cell types tested

FIGS. 10A and 10B show that OSMI-1 is not cytotoxic and does not trigger apoptotic signaling. CHO cells were plated and grown in a 96-well plate to 50-60% confluency and subsequently treated with vehicle (DMSO), 50 μM OSMI-1 (0.5% final vehicle concentration) or 10 μM Staurosporine for the indicated time. After treatment, cells were incubated and analyzed with ApoTox-Glo reagent (Promega) according to the manufacturer's instructions. Cell viability decreases with time for cells treated with OSMI-1 without a concurrent increase in cytotoxicity or apoptotic signaling. Staurosporine is a positive control for apoptotic signaling.

FIG. 11 shows that OSMI-1 is not cytotoxic and does not trigger apoptotic signaling at early time points. CHO cells were plated and grown in a 96-well plate to 50-60% confluency and subsequently treated with vehicle (DMSO), 50 μM OSMI-1 (0.5% final vehicle concentration) or 30 μg/mL Digitonin for 15 minutes. After treatment, cells were incubated and analyzed with ApoTox-Glo reagent (Promega) according to the manufacturer's instructions. OSMI-1 does not exhibit cytotoxicity (middle panel) or trigger apoptotic signaling (right panel) in CHO cells after a 15 min incubation. Digitonin is a positive control for cytotoxicity.

FIG. 12 shows the effect of OGT inhibitors on cell surface glycans as demonstrated. CHO cells were grown to 50-60% confluency and treated with vehicle (DMSO), 50 uM OGT inhibitors Ac-SS-GlcNAc, or OSMI-1 for 24 hours. After treatment, cells were harvested and cell lysates were immunoblotted with the indicated lectins. For the tested lectins, which recognize a range of glycan structures, there were no major changes upon OSMI-1 treatment. Notably, Ac4-5SGlcNAc treatment results in decreased signal intensity of the lectin PHA-E, left, which recognizes galactosylated N-glycans with bisecting N-acetylglucosamines⁶⁶. While lectin specificity is not always fully characterized, the general consensus in the literature appears to be as follows: ECL has been reported to predominantly recognize Galβ1-4-GlcNAc motifs⁶⁷, PHA-L recognizes bisecting GlcNAcs or β1-6 linkages⁶⁸, GSL-1 recognizes α-Gal and α-GalNAc motifs, and both PNA and DBA have been implicated in β-GalNAc recognition⁶⁹. Lectin specificity, including that shown in FIG. 15, was compiled from the Consortium for Functional Glycomics (www.functionalglycomics.org).

FIG. 13 shows the identification and optimization of OSMI-1. FIG. 13A shows several Q6S -containing hits were identified in a high-throughput screen²⁷ and their IC₅₀ values against sOGT are shown. A 1,280-member library of commercially available Q6S-containing molecules was subsequently screened using an FP displacement assay, and selected hits are shown. Q6S derivatives bearing a phenylglycine residue were some of the best hits in a secondary radiometric capture assay. This scaffold was optimized through medicinal chemistry to OSMI-1. FIG. 13B shows the synthetic route to OSMI-1.

FIG. 14 shows that OSMI-1 inhibits OGT in vitro. FIG. 14A shows that OSMI-1 inhibits OGT activity in a dose-dependent manner. FIG. 14B shows that, when using fixed saturating concentrations of GST-Nup62 (protein acceptor), the Vₘₐₓ changes as a function of OSMI-1 concentration, suggesting that it is not competitive with respect to UDP-GlcNAc (See also FIG. 16).

FIG. 15 shows that OSMI-1 inhibits OGT in vivo and does not significantly perturb cell-surface glycan structures. FIG. 15A shows lysates from CHO cells, untreated or treated with either OSMI-1 or Ac4-5SGlcNAc, at 50 μM, immunoblotted for global O-GlcNAc. A full RL2 blot is shown in FIG. 17B. FIG. 15B shows that markers of OGT inhibition include a mass shift of Nup62 and a decrease in OGA levels while OGT levels remain unchanged. FIG. 15C shows that lectins ConA, LCA and jacalin (JAC) can recognize extracellular glycan structures, which should not be affected by a specific inhibitor of OGT. In FIG. 15D, lysates from cells, untreated or treated, at 50 μM, with either OSMI-1 or Ac4-5SGlcNAc, were probed with lectins ConA (left), LCA (middle) and JAC (right).

FIG. 16 shows the In vitro inhibition of OGT by OSMI-1 and UDP-5SGlcNAc. FIG. 16A shows a radiometric peptide capture assay, with UDP-¹⁴C-GlcNAc as the sugar donor, afforded the IC₅₀ values listed. This assay used 6μM UDP-GlcNAc and 18 μM GST-Nup62. FIG. 16B shows a luminescence-based coupled enzyme assay (UDP-Glo; Promega), employing UDP-GlcNAc at 40 μM and 125 μM peptide substrate, CKII3K. FIG. 16C shows the use of various UDP-GlcNAc concentrations (2, 5, 20, 40, 80, 160 and 320 μM) in the UDP-Glo assay showed little change in IC₅₀ value of OSMI-1. FIG. 16D shows the IC₅₀ values, obtained from FIG. 16C, plotted as a function of donor concentration. Values are listed as mean IC₅₀ (95% CI), with curved solid lines demarking the 95% CI for the linear regression. The equation of the line of best fit is also shown. A dashed line, with its corresponding equation is also shown. This line is derived from Equation 1, below, with Kᵢ being obtained as the Y-intercept from the aforementioned linear regression (had OSMI-1 been a competitive inhibitor) and a Kₘ [for UDP-GlcNAc] of 2.3 μM⁴⁶. A robust variable-slope non-linear fit was used to obtain IC₅₀ values and Hill slopes in all cases. It is noteworthy that a different aliquot of OSMI-1 was used for the experiments in FIGS. 16C and 16D, which likely accounts for the 2-fold change in potency relative to the above stated 40 μM IC₅₀.

FIG. 17 shows full length RL2 blots displaying molecular weight markers. FIG. 17A shows CHO, HEK-293 HeLa and D283-Med cells (left to right) grown to 50-60% confluency (CHO, HeLa, or D283-Med) or >80% confluency (HEK-293) that were treated with DMSO for 24 hours. After treatment, cells were harvested and cell lysates were immunoblotted with RL2. Markers shown were used to assign molecular weights on RL2 blots shown in FIGS. 15A, 7, 8, and 9A. FIG. 17B presents the full RL2 blot of that shown in FIG. 15A.

FIG. 18 shows that PG34, a close structural analog of OSMI-1, does not inhibit OGT. FIG. 18A shows a comparison of OSMI-1 and PG34 in terms of molecular structure, cellular permeability (as determined by PAMPA) and in vitro potency (IC₅₀ values determined by radiometric capture assay). PG34, while similar in structure to OSMI-1, is a poor OGT inhibitor in vitro. FIG. 18B shows that PG34 does not detectably inhibit OGT in cells. CHO cells grown to 50-60% confluency and treated with PG34 at the indicated concentrations for 24 hours. After treatment, cells were harvested and cell lysates were immunoblotted with the indicated antibodies. At concentrations of PG34 up to 50 μM, there was no reduction in global O-GlcNAc levels. FIG. 18C shows that both OSMI-1 and PG34 have similar effects on cell viability, cytotoxicity, and apoptotic signaling. CHO cells were plated in a 96-well plate (5,000 cells/well) and grown for 24 hours. Cells were then treated with either DMSO alone or increasing concentrations (0.5-50 μM) of OSMI-1 or PG34 and grown for an additional 24 hours. After treatment, cells were incubated with ApoTox-Glo reagent (Promega) and analyzed according to the manufacturer's instructions. All measurements were performed in triplicate and results are normalized to represent fold-change from DMSO-only-treated cells. Neither compound displayed an effect on apoptotic signaling, but at the highest concentration both compounds have a moderate effect on cell viability. Bars represent the change at the following concentrations: 0, 0.5, 1.0, 5.0, 10.0, 25.0 and 50.0 μM (left to right), and with 0 and 50 μM being marked with ticks. For clarity, this is only shown for the first group of data.

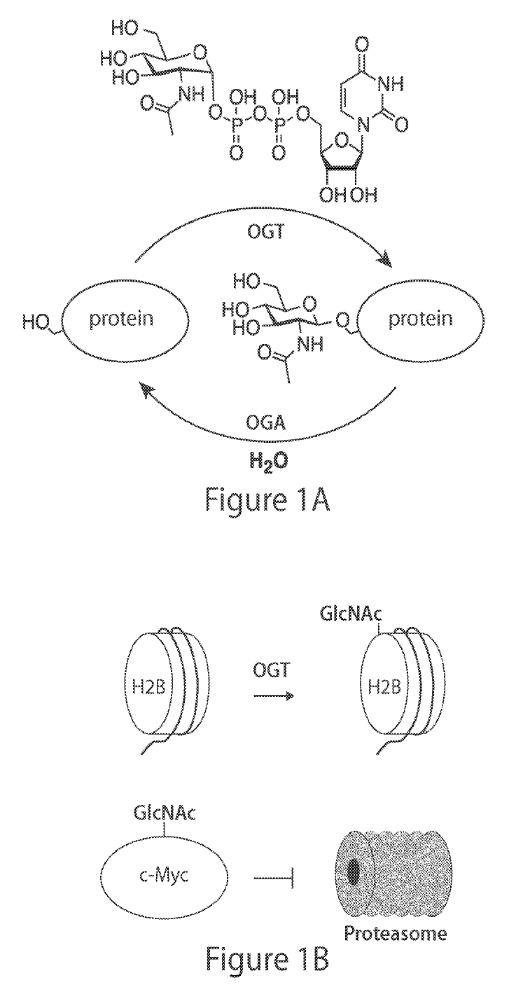

### DETAILED DESCRIPTION OF CERTAIN EMBODIMENTS OF THE INVENTION

The present invention provides compounds of Formula (I), which have been found to be inhibitors of O-GlcNAc transferase. The inventive compounds typically include a quinolinone or quinazolinedione core as shown herein. The compounds of the present invention are useful in the treatment of OGT-related diseases or disorders. Specifically, the compounds are useful in the treatment of metabolic diseases such as diabetes and complications thereof, neurological diseases, proliferative diseases such as cancers, and autoimmune diseases, and inflammatory diseases. The present invention also provides pharmaceutical compositions, kits, and methods of using the inventive compounds for the treatment of various diseases. Methods of preparing the compounds of Formula (I) are also provided.

### Compounds

Compounds of the present invention are provided as shown in Formula (I) In certain embodiments, the compounds have an IC₅₀ of less than approximately 100 μM, e.g., less than approximately 10 μM, less than approximately 1 μM, less than approximately 0.1 μM, or less than approximately 0.01 μM. The inventive compounds may be useful in the treatment of a variety of diseases. In certain embodiments, the compounds are useful in the treatment of metabolic diseases such as diabetes, and complications thereof, and insulin resistance. Certain compounds are also useful in treating neurological diseases, such as neurodegenerative diseases. In certain embodiments, the compounds are useful in the treatment of proliferative disease including certain types of cancers and benign neoplasms. In other embodiments, the compounds are useful in treating autoimmune diseases or inflammatory diseases.

In certain embodiments, the invention provides a compound of Formula (I):

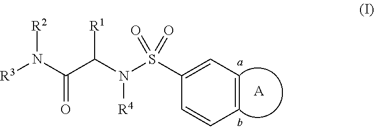

or a pharmaceutically acceptable salt thereof,
wherein

Ring A is of the formula

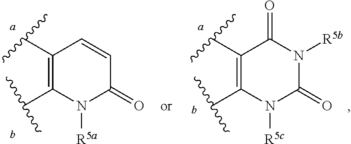

wherein a and b indicate the points of attachment to the phenyl ring;

R¹ is n-butyl, thiophene, —CH₂-Ph, cyclohexyl, or of the formula:

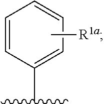

R^{1a} is hydrogen, halogen, —OR⁰, or optionally substituted C₁₋₄ alkyl;

R⁰ is hydrogen or C₁₋₄ alkyl;

each of R² and R³ is independently hydrogen, optionally substituted C₁₋₄ alkyl, optionally substituted thiophenyl-C₁₋₄ alkylene, optionally substituted phenyl-C₁₋₄ alkylene, or optionally substituted furanyl-C₁₋₄ alkylene;

R⁴ is hydrogen, optionally substituted C₁₋₆ alkyl, or a nitrogen protecting group;

each of R^{5a}, R^{5b}, and R^{5c} is independently hydrogen, optionally substituted C₁₋₆ alkyl, or a nitrogen protecting group;

R¹ and R⁴ may optionally be taken together with the intervening nitrogen to form an optionally substituted heteroaryl or optionally substituted heterocycle;

R² and R³ may optionally be taken together with the intervening nitrogen to form an optionally substituted six-membered heterocycle.

In certain embodiments, the invention provides a compound of the following formula:

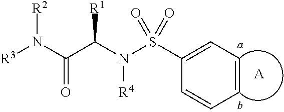

or a pharmaceutically acceptable salt thereof.

In certain embodiments, the invention provides a compound of the following formula:

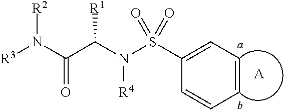

or a pharmaceutically acceptable salt thereof.

In certain embodiments, the compound of Formula (I) is of one of the following compounds:

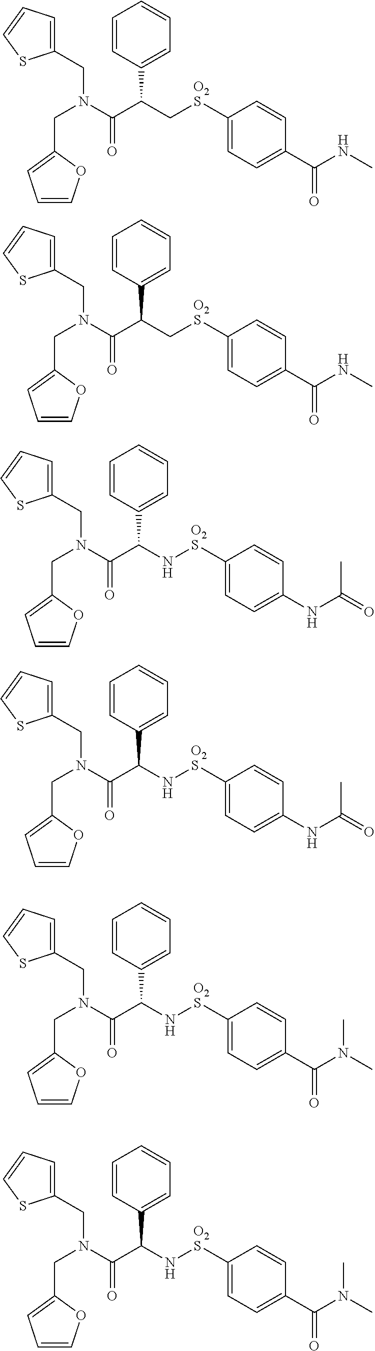

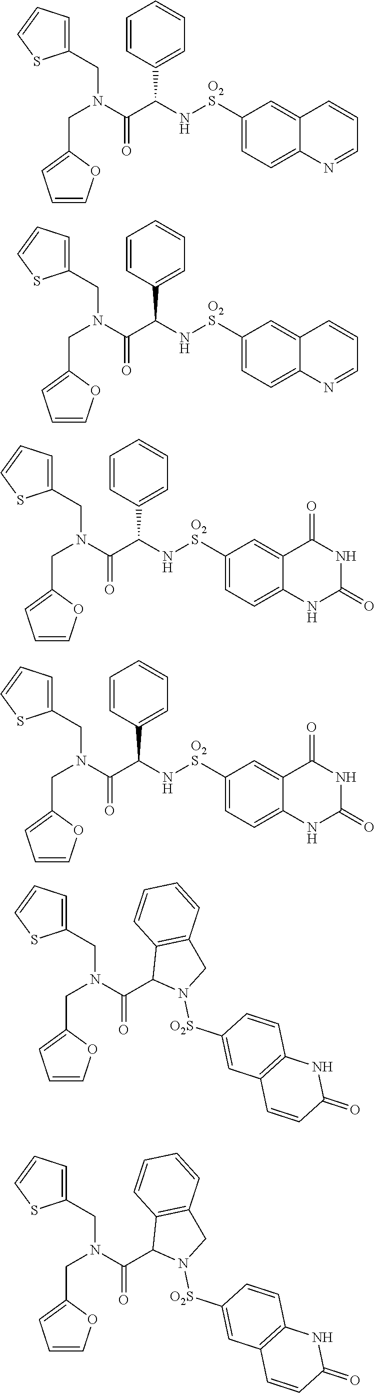

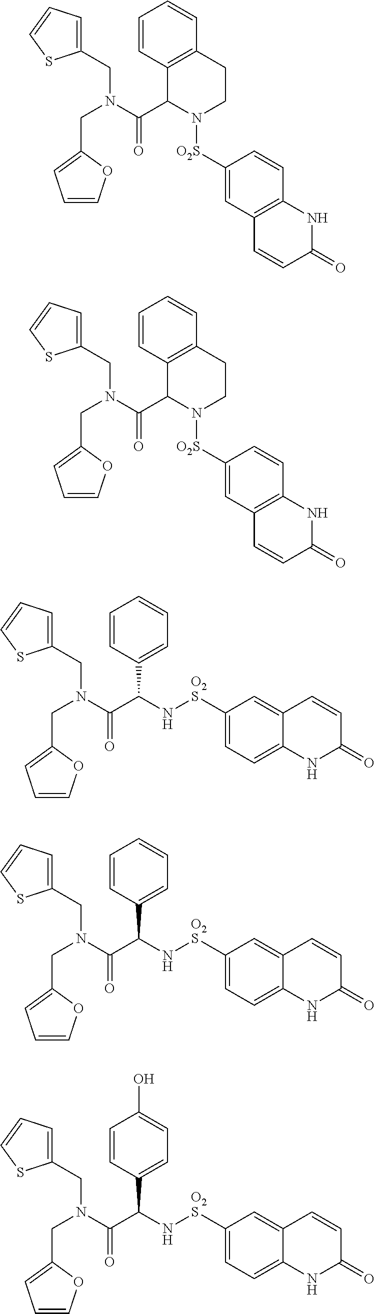 [SMILES: O=C(C1c2ccccc2CCN1S(=O)(=O)c1ccc2[nH]c(=O)ccc2c1)N(Cc1ccco1)Cc1cccs1 ; O=C(C1c2ccccc2CCN1S(=O)(=O)c1ccc2[nH]c(=O)ccc2c1)N(Cc1ccco1)Cc1cccs1 ; O=C([C@@H](NS(=O)(=O)c1ccc2[nH]c(=O)ccc2c1)c1ccccc1)N(Cc1ccco1)Cc1cccs1 ; O=C([C@H](NS(=O)(=O)c1ccc2[nH]c(=O)ccc2c1)c1ccccc1)N(Cc1ccco1)Cc1cccs1 ; O=C([C@H](NS(=O)(=O)c1ccc2[nH]c(=O)ccc2c1)c1ccc(O)cc1)N(Cc1ccco1)Cc1cccs1]

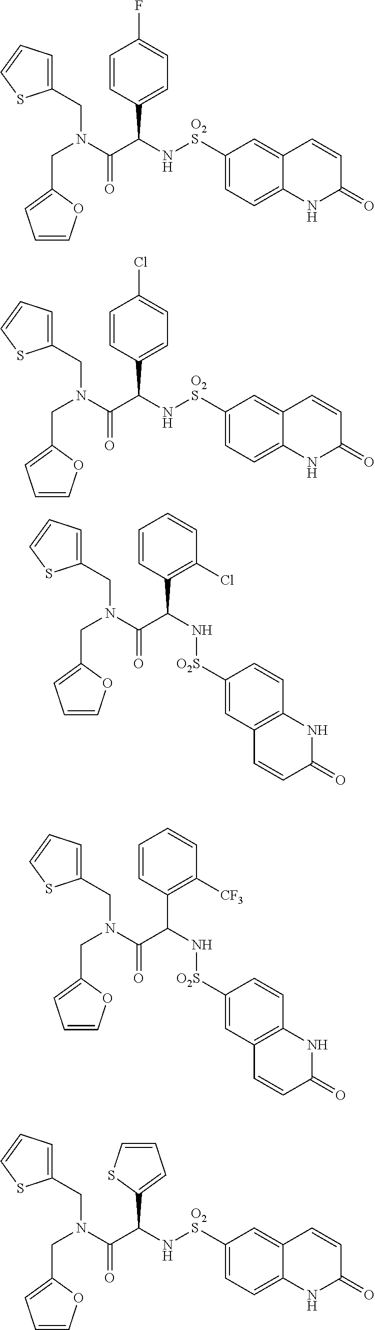

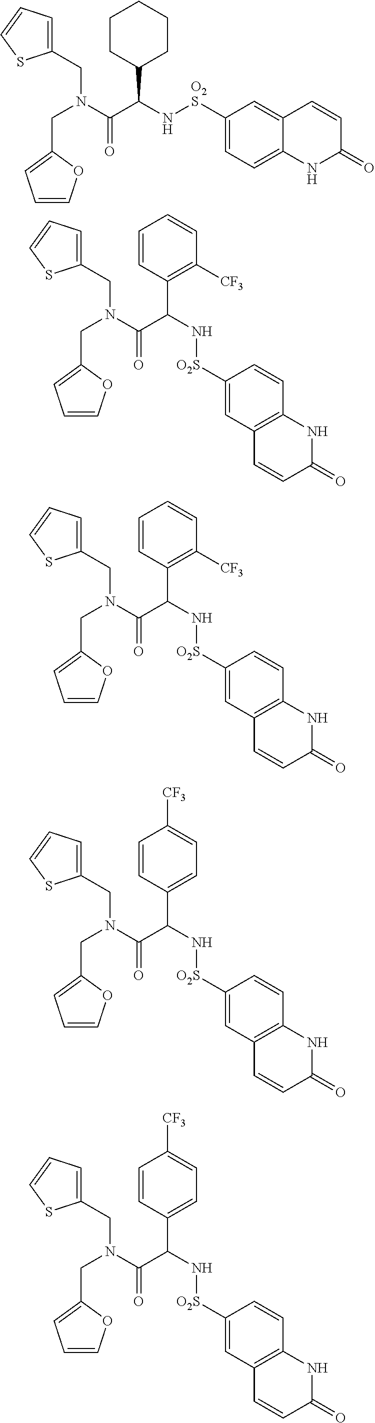

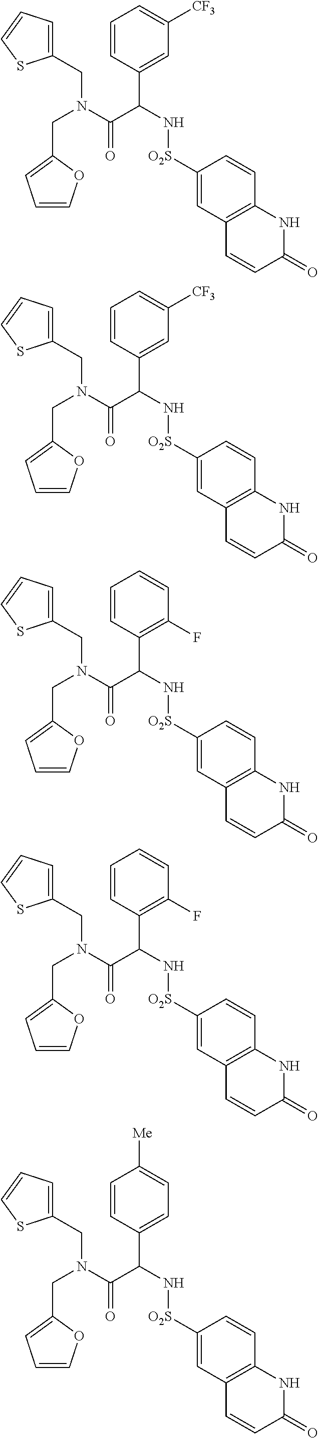

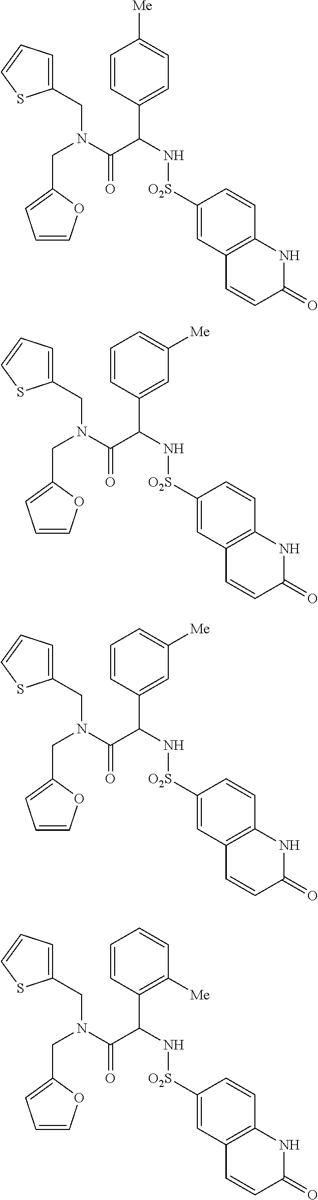 [SMILES: Cc1ccc(C(NS(=O)(=O)c2ccc3[nH]c(=O)ccc3c2)C(=O)N(Cc2ccco2)Cc2cccs2)cc1 ; Cc1cccc(C(NS(=O)(=O)c2ccc3[nH]c(=O)ccc3c2)C(=O)N(Cc2ccco2)Cc2cccs2)c1 ; Cc1cccc(C(NS(=O)(=O)c2ccc3[nH]c(=O)ccc3c2)C(=O)N(Cc2ccco2)Cc2cccs2)c1 ; Cc1ccccc1C(NS(=O)(=O)c1ccc2[nH]c(=O)ccc2c1)C(=O)N(Cc1ccco1)Cc1cccs1]

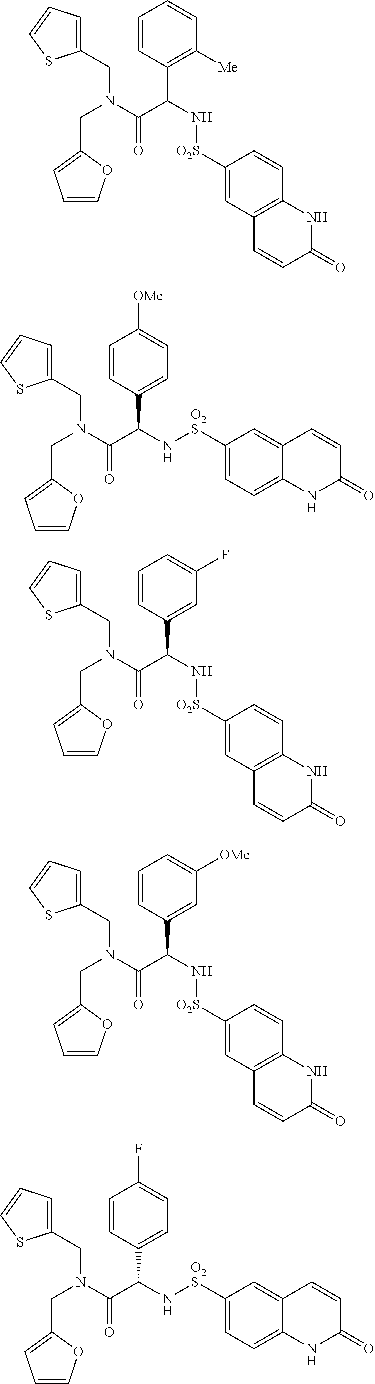

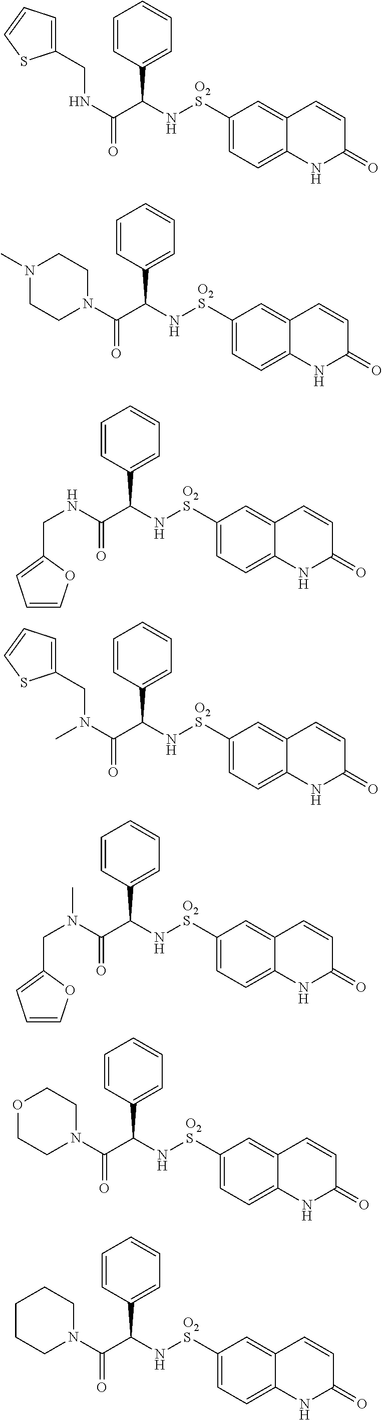

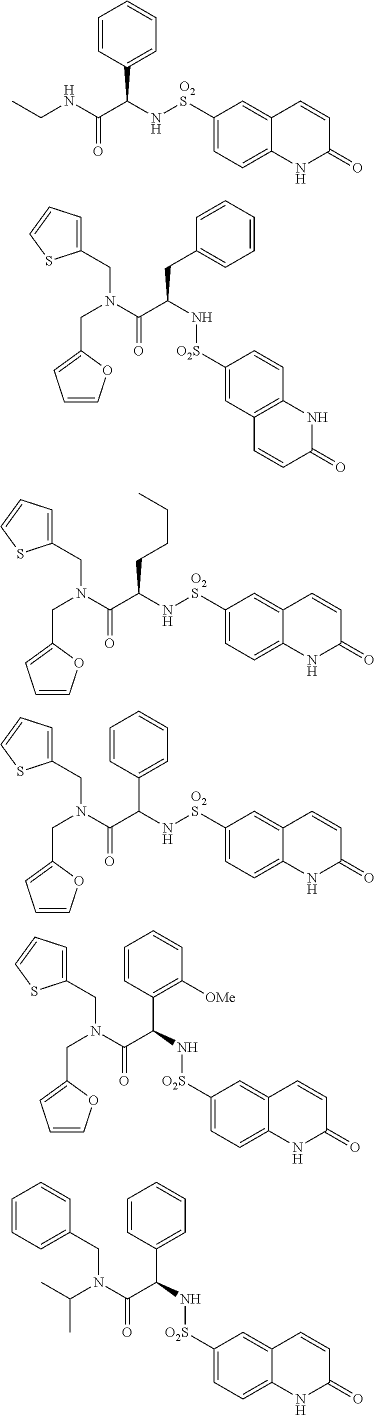

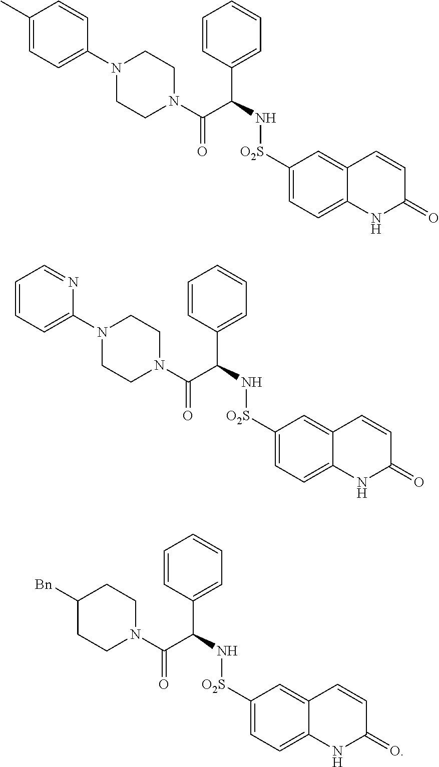 [SMILES: Cc1ccc(N2CCN(C(=O)[C@H](NS(=O)(=O)c3ccc4[nH]c(=O)ccc4c3)c3ccccc3)CC2)cc1 ; O=C([C@H](NS(=O)(=O)c1ccc2[nH]c(=O)ccc2c1)c1ccccc1)N1CCN(c2ccccn2)CC1 ; O=C([C@H](NS(=O)(=O)c1ccc2[nH]c(=O)ccc2c1)c1ccccc1)N1CCC(Cc2ccccc2)CC1]

In certain embodiments, the compound of Formula (I) is not one of the following compounds:

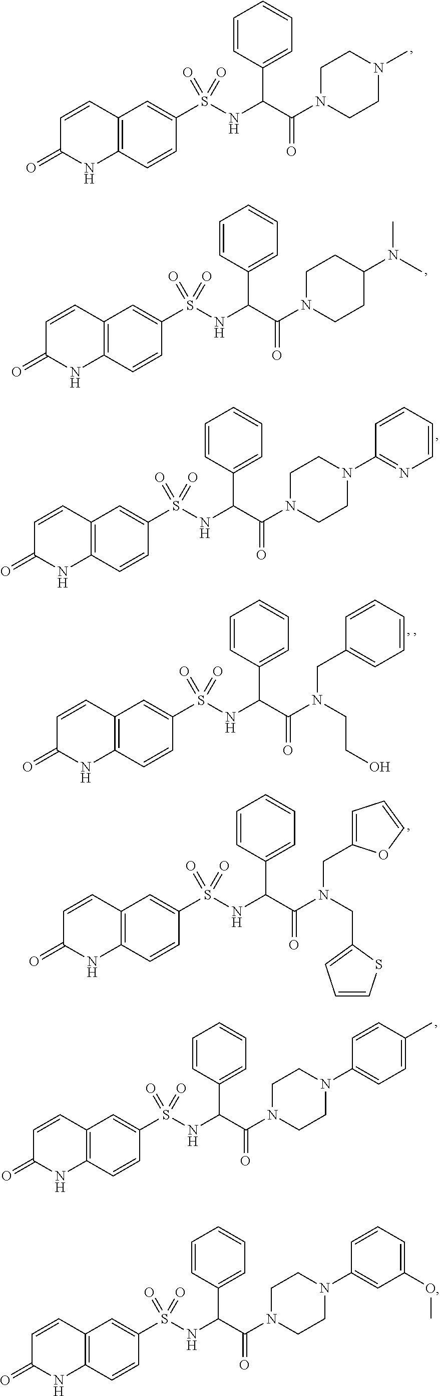

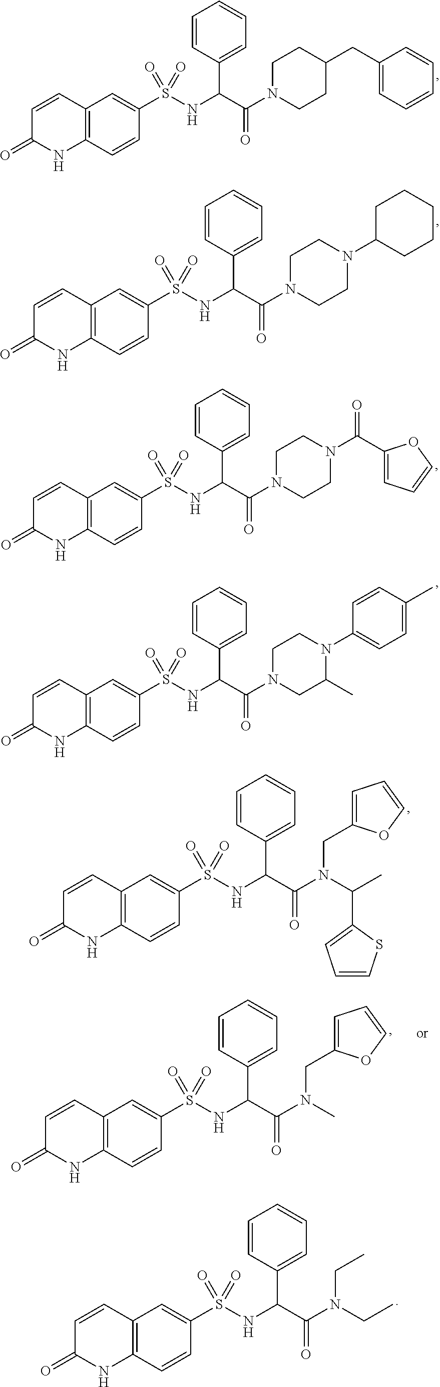

As generally defined herein, R¹ is n-butyl, thiophene, —CH₂-Ph, cyclohexyl, or of the formula:

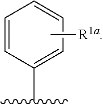

In some embodiments, R¹ is n-butyl. In some embodiments, R¹ is thiophene. In some embodiments, R¹ is of the formula

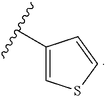 [SMILES: *c1ccsc1]

In some embodiments, R¹ is of the formula

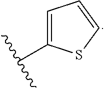 [SMILES: *c1cccs1]

In some embodiments, R¹ is —CH₂-Ph. In some embodiments, R¹ is cyclohexyl. In some embodiments, R¹ is of the formula

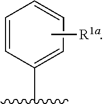

As generally defined herein, R^{1a} is hydrogen, halogen, —OR⁰, or optionally substituted C₁₋₄ alkyl. In some embodiments, R^{1a} is hydrogen. In some embodiments, R^{1a} is halogen, —OR⁰, or optionally substituted C₁₋₄ alkyl. In some embodiments, R^{1a} is halogen. In some embodiments, R^{1a} is F. In some embodiments, R^{1a} is Cl. In some embodiments, R^{1a} is optionally substituted C₁₋₄ alkyl. In some embodiments, R^{1a} is unsubstituted C₁₋₄ alkyl. In some embodiments, R^{1a} is methyl or ethyl. In some embodiments, R^{1a} is substituted C₁₋₄ alkyl. In some embodiments, R^{1a} is CF₃. In some embodiments, R^{1a} is —OR⁰, wherein R⁰ is hydrogen or C₁₋₄ alkyl. In some embodiments, R^{1a} is —OH. In some embodiments, R^{1a} is —OR⁰, wherein R⁰ is C₁₋₄ alkyl. In some embodiments, R^{1a} is —OCH₃. In some embodiments, R^{1a} is —OC₂H₅. In some embodiments, R^{1a} is of the formula

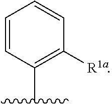

In some embodiments, R^{1a} is of the formula

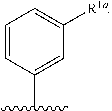

In some embodiments, R^{1a} is of the formula

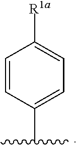

In some embodiments, R¹ is of one of the following formulae:

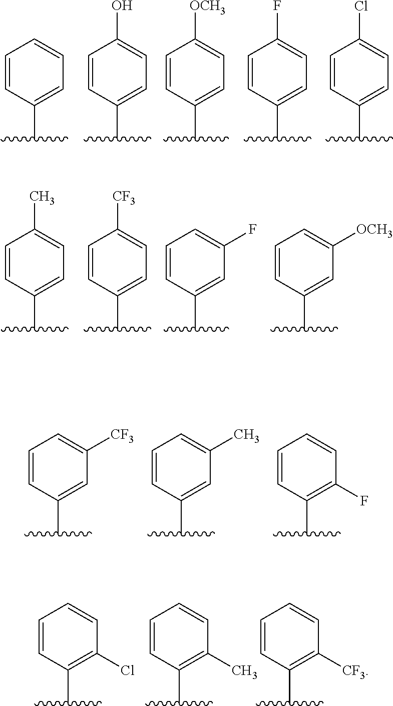

In some embodiments, R¹ is of one of the following formulae:

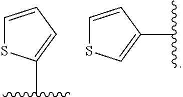

In certain embodiments, the compound of Formula (I) is of Formula (II):

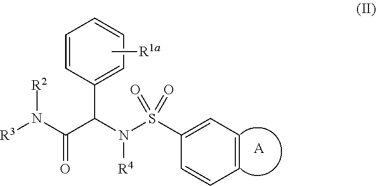

or a pharmaceutically acceptable salt thereof.

In certain embodiments, the compound of Formula (II) is of Formula (II-a) or (II-b):

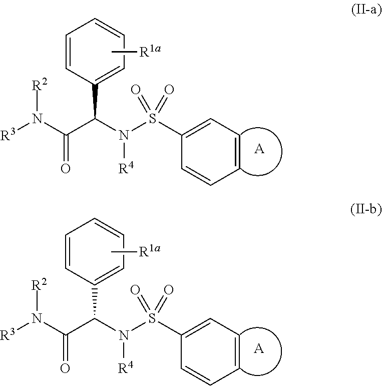

or a pharmaceutically acceptable salt thereof.

As generally defined here, R² is hydrogen; optionally substituted C₁₋₄ alkyl; optionally substituted thiophenyl-C₁₋₄ alkylene; optionally substituted phenyl-C₁₋₄ alkylene; or optionally substituted furanyl-C₁₋₄ alkylene. In certain embodiments, R² is hydrogen; optionally substituted C₁₋₄ alkyl; optionally substituted thiophenyl-C₁₋₄ alkylene; or optionally substituted furanyl-C₁₋₄ alkylene. In some embodiments, R² is hydrogen. In some embodiments, R² is optionally substituted C₁₋₄ alkyl. In some embodiments, R² is unsubstituted C₁₋₄ alkyl. In some embodiments, R² is substituted C₁₋₄ alkyl. In some embodiments, R² is methyl. In some embodiments, R² is substituted methyl. In some embodiments, R² is ethyl. In some embodiments, R² is optionally substituted thiophenyl-C₁₋₄ alkylene, optionally substituted phenyl-C₁₋₄ alkylene, or optionally substituted furanyl-C₁₋₄ alkylene. In some embodiments, R² is unsubstituted thiophenyl-C₁₋₄ alkylene, unsubstituted phenyl-C₁₋₄ alkylene, or unsubstituted furanyl-C₁₋₄ alkylene. In some embodiments, R² is substituted thiophenyl-C₁₋₄ alkylene, substituted phenyl-C₁₋₄ alkylene, or substituted furanyl-C₁₋₄ alkylene. In some embodiments, R² is optionally substituted thiophenyl —CH₂—. In some embodiments, R² is optionally substituted phenyl —CH₂—. In some embodiments, R² is optionally substituted furanyl-CH₂—. In some embodiments, R² is thiophene-CH₂—. In some embodiments, R² is of one of the following formulae:

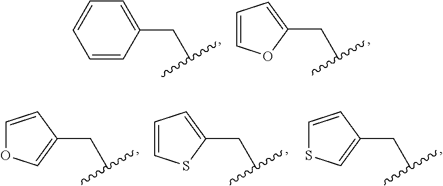 [SMILES: *Cc1ccccc1 ; *Cc1ccco1 ; *Cc1ccoc1 ; *Cc1cccs1 ; *Cc1ccsc1]

As generally defined here, R³ is hydrogen; optionally substituted C₁₋₄ alkyl; optionally substituted thiophenyl-C₁₋₄ alkylene; optionally substituted phenyl-C₁₋₄ alkylene; or optionally substituted furanyl-C₁₋₄ alkylene. In certain embodiments, R³ is hydrogen; optionally substituted C₁₋₄ alkyl; optionally substituted thiophenyl-C₁₋₄ alkylene; or optionally substituted furanyl-C₁₋₄ alkylene. In some embodiments, R³ is hydrogen. In some embodiments, R³ is optionally substituted C₁₋₄ alkyl. In some embodiments, R³ is unsubstituted C₁₋₄ alkyl. In some embodiments, R³ is substituted C₁₋₄ alkyl. In some embodiments, R³ is methyl. In some embodiments, R³ is ethyl. In some embodiments, R³ is optionally substituted thiophenyl-C₁₋₄ alkylene, optionally substituted phenyl-C₁₋₄ alkylene, or optionally substituted furanyl-C₁₋₄ alkylene. In some embodiments, R³ is unsubstituted thiophenyl-C₁₋₄ alkylene, unsubstituted phenyl-C₁₋₄ alkylene, or unsubstituted furanyl-C₁₋₄ alkylene. In some embodiments, R³ is substituted thiophenyl-C₁₋₄alkylene, substituted phenyl-C₁₋₄ alkylene, or substituted furanyl-C₁₋₄alkylene. In some embodiments, R³ is optionally substituted thiophenyl-CH₂, optionally substituted phenyl-CH₂, or optionally substituted furanyl-CH₂. In some embodiments, R³ is optionally substituted furanyl-CH₂—. In some embodiments, R³ is optionally substituted thiophenyl-CH₂—. In some embodiments, R³ is optionally substituted phenyl-CH₂—. In some embodiments, R³ is of one of the following formulae:

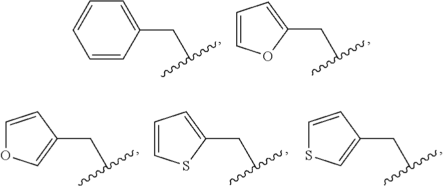 [SMILES: *Cc1ccccc1 ; *Cc1ccco1 ; *Cc1ccoc1 ; *Cc1cccs1 ; *Cc1ccsc1]

In some embodiments, each of R² and R³ is independently optionally substituted thiophenyl-C₁₋₄ alkylene, optionally substituted phenyl-C₁₋₄ alkylene, or optionally substituted furanyl-C₁₋₄ alkylene. In some embodiments, each of R² and R³ is independently optionally substituted furanyl-CH₂—, optionally substituted phenyl-CH₂—, or optionally substituted thiophenyl-CH₂—. In some embodiments, each of R² and R³ is independently optionally substituted thiophenyl-C₁₋₄ alkylene or optionally substituted furanyl-C₁₋₄ alkylene. In some embodiments, each of R² and R³ is independently optionally substituted furanyl-CH₂— or optionally substituted thiophenyl-CH₂—. In some embodiments, each of R² and R³ is independently

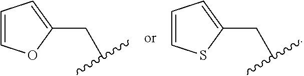 [SMILES: *Cc1ccco1 ; *Cc1cccs1]

In some embodiments, R² is

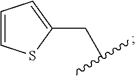 [SMILES: *Cc1cccs1]

### and R³ is

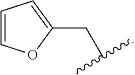 [SMILES: *Cc1ccco1]

In some embodiments, R² hydrogen; and R³ is C₁₋₄ alkyl. In certain embodiments, R² is hydrogen; and R³ is methyl, ethyl, iso-propyl, or n-propyl.

In some embodiments, R² is C₁₋₄ alkyl; and R³ is optionally substituted thiophenyl-C₁₋₄ alkylene, optionally substituted phenyl-C₁₋₄ alkylene, or optionally substituted furanyl-C₁₋₄ alkylene. In some embodiments, R² is methyl, ethyl, n-propyl, or iso-propyl; and R³ is optionally substituted thiophenyl-C₁₋₄ alkylene, optionally substituted phenyl-C₁₋₄ alkylene, or optionally substituted furanyl-C₁₋₄ alkylene. In some embodiments, R² is methyl; and R³ is optionally substituted thiophenyl-C₁₋₄ alkylene, optionally substituted phenyl-C₁₋₄ alkylene, or optionally substituted furanyl-C₁₋₄ alkylene. In some embodiments, R² is ethyl; and R³ is optionally substituted thiophenyl-C₁₋₄ alkylene, optionally substituted phenyl-C₁₋₄ alkylene, or optionally substituted furanyl-C₁₋₄ alkylene. In some embodiments, R² is n-propyl; and R³ is optionally substituted thiophenyl-C₁₋₄ alkylene, optionally substituted phenyl-C₁₋₄ alkylene, or optionally substituted furanyl-C₁₋₄ alkylene. In some embodiments, R² is iso-propyl; and R³ is optionally substituted thiophenyl-C₁₋₄ alkylene, optionally substituted phenyl-C₁₋₄ alkylene, or optionally substituted furanyl-C₁₋₄ alkylene. In some embodiments, R² is methyl or ethyl; and R³ is thiophenyl-C₁₋₄ alkylene or optionally substituted furanyl-C₁₋₄ alkylene. In some embodiments, R² is methyl or ethyl; and R³ is optionally substituted furanyl-CH₂— or optionally substituted thiophenyl-CH₂—. In some embodiments, R² is iso-propyl; and R³ is optionally substituted phenyl-CH₂—. In some embodiments, R² is methyl, ethyl, n-propyl, or iso-propyl; and R³ is

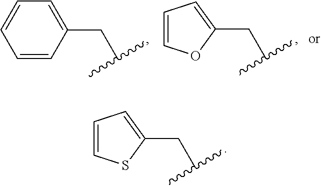 [SMILES: *Cc1ccccc1 ; *Cc1ccco1 ; *Cc1cccs1]

In some embodiments, R² is methyl or ethyl; and R³ is

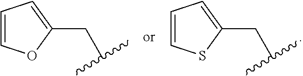

In some embodiments, R² and R³ are taken together with the intervening nitrogen to form optionally substituted six-membered heterocycle. In some embodiments, R² and R³ are taken together with the intervening nitrogen to form optionally substituted piperidinyl, piperazinyl, or morpholinyl ring. In some embodiments, R² and R³ are taken together with the intervening nitrogen to form unsubstituted piperidinyl, piperazinyl, or morpholinyl ring.

In some embodiments, R² and R³ are taken together with the intervening nitrogen to form the formula:

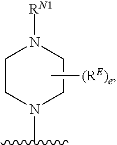

wherein

each instance of R^{E} is independently selected from the group consisting of hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted alkoxy, an optionally substituted amino group, or optionally substituted acyl;

e is 0, or an integer of 1 to 6; and

R^{N1} is optionally substituted alkyl, optionally substituted aryl, optionally substituted carbocyclyl, optionally substituted heterocyclyl, optionally substituted heteroaryl, or a nitrogen protecting group.

In certain embodiments, R^{N1} is optionally substituted C₁₋₄ alkyl, optionally substituted phenyl, optionally substituted 5-membered or 6-membered carbocyclyl, optionally substituted 5-membered or 6-membered heterocyclyl, or optionally substituted 5-membered or 6-membered heteroaryl. In certain embodiments, R^{N1} is optionally substituted C₁₋₄ alkyl. In certain embodiments, R^{N1} is unsubstituted C₁₋₄ alkyl. In certain embodiments, R^{N1} is methyl or ethyl. In certain embodiments, R^{N1} is optionally substituted aryl. In certain embodiments, R^{N1} is optionally substituted phenyl. In certain embodiments, R^{N1} is unsubstituted phenyl. In certain embodiments, R^{N1} is substituted phenyl. In certain embodiments, R^{N1} is mono-substituted phenyl. In certain embodiments, R^{N1} is o-methyl phenyl, p-methyl phenyl, or m-methyl phenyl. In certain embodiments, R^{N1} is di-substituted phenyl. In certain embodiments, R^{N1} is tri-substituted phenyl. In certain embodiments, R^{N1} is tetra-substituted phenyl. In certain embodiments, R^{N1} is optionally substituted carbocyclyl. In certain embodiments, R^{N1} is optionally substituted 6-membered carbocyclyl. In certain embodiments, R^{N1} is optionally substituted 5-membered carbocyclyl. In certain embodiments, R^{N1} is optionally substituted 6-membered heterocyclyl. In certain embodiments, R^{N1} is optionally substituted 5-membered heterocyclyl. In certain embodiments, R^{N1} is optionally substituted 6-membered heterocyclyl. In certain embodiments, R^{N1} is optionally substituted 5-membered heteroaryl. In certain embodiments, R^{N1} is optionally substituted 5-membered heteroaryl with one heteroatom selected from the group consisting of N, O, and S. In certain embodiments, R^{N1} is optionally substituted 5-membered heteroaryl with two heteroatoms selected from the group consisting of N, O, and S. In certain embodiments, R^{N1} is optionally substituted 6-membered heteroaryl. In certain embodiments, R^{N1} is optionally substituted 6-membered heteroaryl with one heteroatom selected from the group consisting of N, O, and S. In certain embodiments, R^{N1} is optionally substituted 6-membered heteroaryl with two heteroatoms selected from the group consisting of N, O, and S. In certain embodiments, R^{N1} is optionally substituted pyridinyl. In certain embodiments, R^{N1} is unsubstituted pyridinyl. In certain embodiments, R^{N1} is substituted pyridinyl. In certain embodiments, R^{N1} is of one of the formulae:

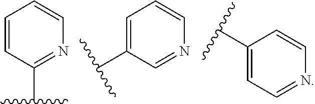

In certain embodiments, R^{N1} is of the formula:

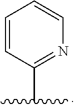 [SMILES: *c1ccccn1]

In some embodiments, R² and R³ are taken together with the intervening nitrogen to form one of the following formulae:

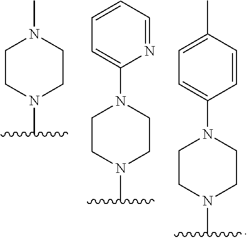

In some embodiments, R² and R³ are taken together with the intervening nitrogen to form the formula:

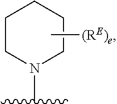

wherein each instance of R^{E} is independently selected from the group consisting of hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted alkoxy, an optionally substituted amino group, or optionally substituted acyl; and e is 0, or an integer of 1 to 6. In certain embodiments, e is 0.

In certain embodiments, e is 1. In certain embodiments, e is 2. In certain embodiments, e is 3. In certain embodiments, e is 4. In certain embodiments, e is 5. In certain embodiments, R^{E} is hydrogen, halogen, or optionally substituted C₁₋₆ alkyl. In some embodiments, R^{E} is hydrogen. In some embodiments, R^{E} is halogen. In certain embodiments, R^{E} is F. In certain embodiments, R^{E} is Cl. In certain embodiments, R^{E} is Br. In certain embodiments, R^{E} is I. In certain embodiments, R^{E} is optionally substituted C₁₋₆ alkyl. In certain embodiments, R^{E} is substituted C₁₋₆ alkyl. In certain embodiments, R^{E} is optionally substituted phenyl-C₁₋₆ alkyl. In certain embodiments, R^{E} is —Bn. In certain embodiments, R^{E} is unsubstituted C₁₋₆ alkyl. In certain embodiments, R^{E} is methyl. In certain embodiments, R^{E} is ethyl.

In some embodiments, R² and R³ are taken together with the intervening nitrogen to form one of the formulae:

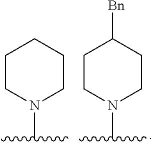

In some embodiments, R² and R³ are taken together with the intervening nitrogen to form the formula:

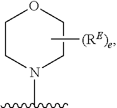

wherein R^{E} and e are as defined herein. In certain embodiments, R² and R³ are taken together with the intervening nitrogen to form the formula:

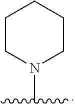 [SMILES: *N1CCCCC1]

In certain embodiments, the compound of Formula (I) is of Formula (III):

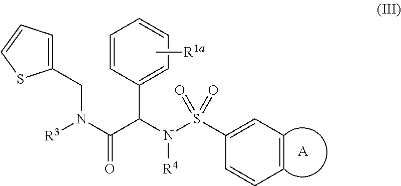

or a pharmaceutically acceptable salt thereof.

In certain embodiments, the compound of Formula (III) is of Formula (III-a):

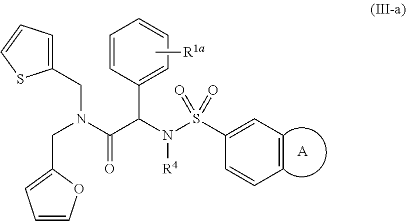

or a pharmaceutically acceptable salt thereof.

In certain embodiments, the compound of Formula (III-a) is of one of the following formulae:

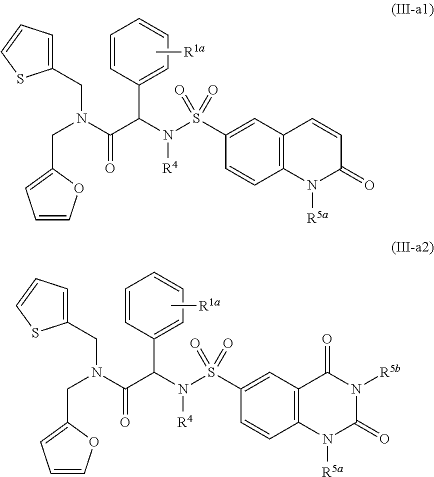

or a pharmaceutically acceptable salt thereof.

In certain embodiments, the compound of Formula (III-a) is of one of the following formulae:

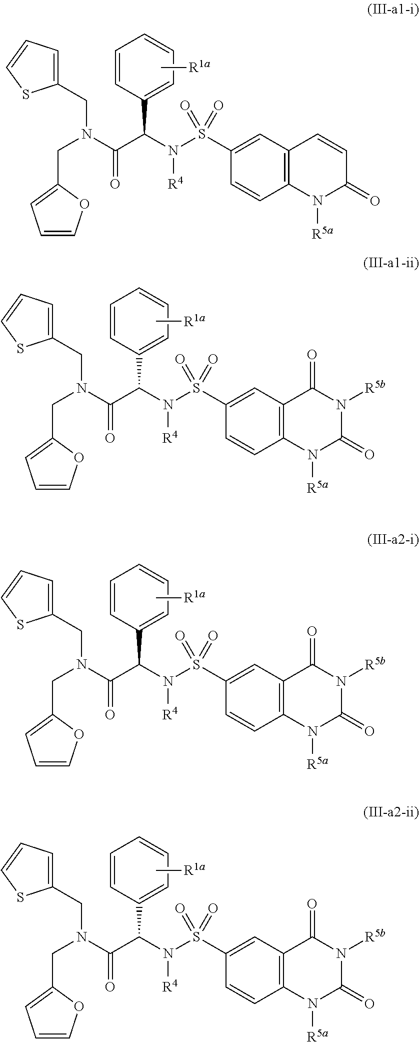

or a pharmaceutically acceptable salt thereof.

In certain embodiments, the compound of Formula (III-a) is of one of the following formulae:

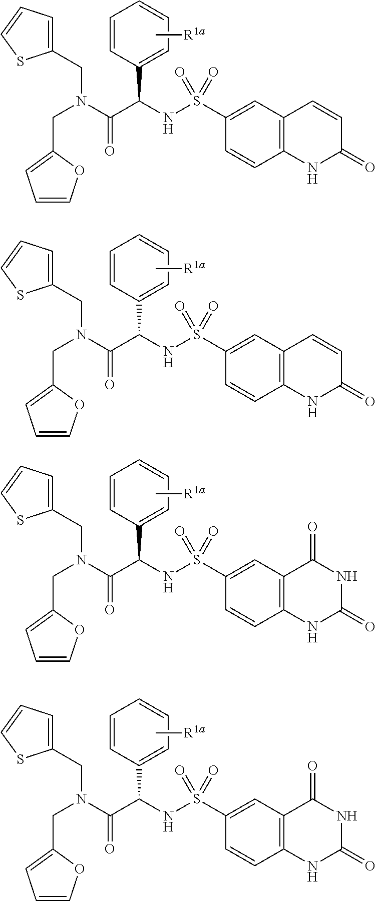

or a pharmaceutically acceptable salt thereof.

In certain embodiments, the compound of Formula (I) is of the formula:

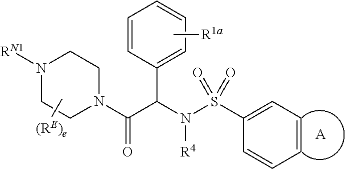

or a pharmaceutically acceptable salt thereof, wherein R^{N1} is as defined herein.

In certain embodiments, the compound of Formula (I) is of the formula:

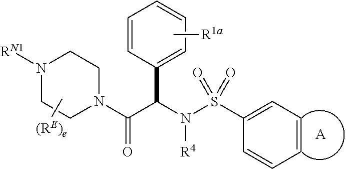

or a pharmaceutically acceptable salt thereof, wherein R^{N1} is as defined herein.

In certain embodiments, the compound of Formula (I) is of the formula:

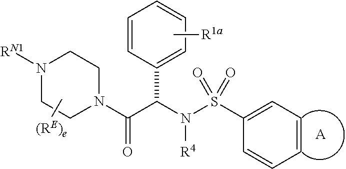

or a pharmaceutically acceptable salt thereof, wherein R^{N1} is as defined herein.

In certain embodiments, the compound of Formula (I) is of the formula:

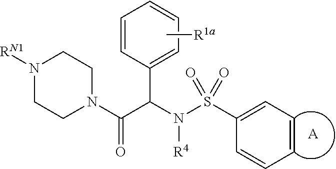

or a pharmaceutically acceptable salt thereof, wherein R^{N1} is as defined herein.

In certain embodiments, the compound of Formula (I) is of the formula:

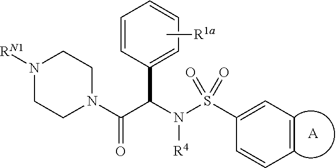

or a pharmaceutically acceptable salt thereof, wherein R^{N1} is as defined herein.

In certain embodiments, the compound of Formula (I) is of the formula:

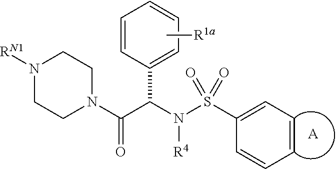

or a pharmaceutically acceptable salt thereof, wherein R^{N1} is as defined herein.

In certain embodiments, the compound of Formula (I) is of the following formula:

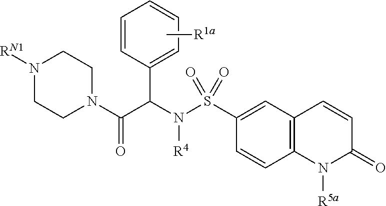

or a pharmaceutically acceptable salt thereof.

In certain embodiments, the compound of Formula (I) is of one of the following formulae:

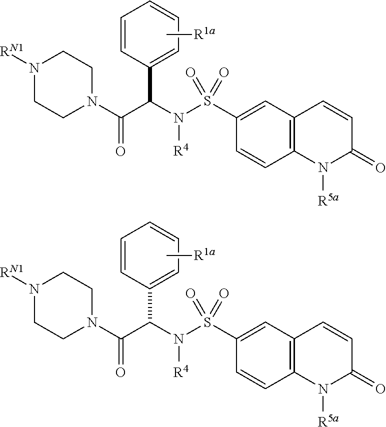

or a pharmaceutically acceptable salt thereof.

In certain embodiments, the compound of Formula (I) is of the following formula:

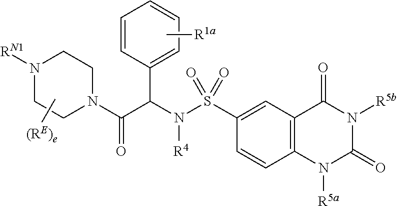

or a pharmaceutically acceptable salt thereof.

In certain embodiments, the compound of Formula (I) is of one of the following formulae:

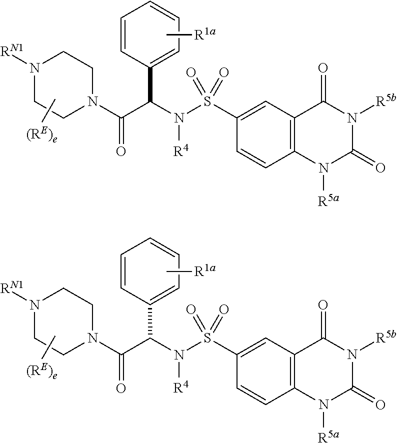

or a pharmaceutically acceptable salt thereof.

In certain embodiments, the compound of Formula (I) is of the following formula:

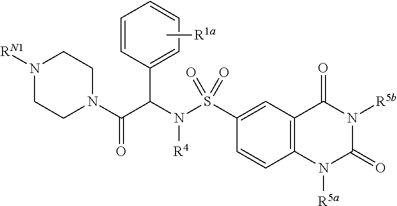

or a pharmaceutically acceptable salt thereof.

In certain embodiments, the compound of Formula (I) is of one of the following formulae:

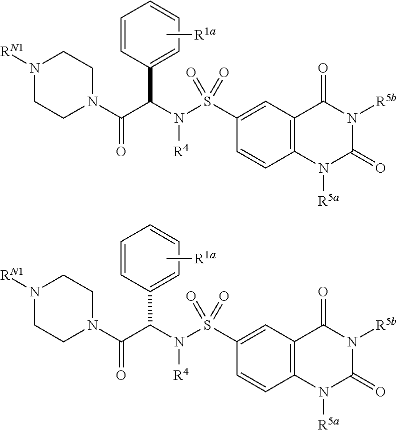

or a pharmaceutically acceptable salt thereof.

In certain embodiments, the compound of Formula (I) is of the formula:

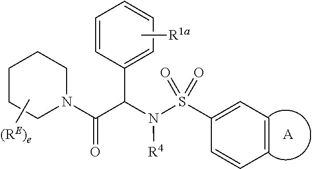

or a pharmaceutically acceptable salt thereof, wherein R^{N1} is as defined herein.

In certain embodiments, the compound of Formula (I) is of the formula:

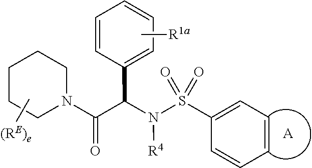

or a pharmaceutically acceptable salt thereof, wherein R^{N1} is as defined herein.

In certain embodiments, the compound of Formula (I) is of the formula:

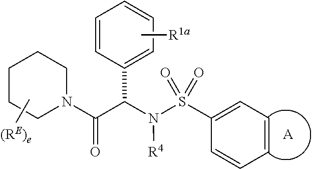

or a pharmaceutically acceptable salt thereof, wherein R^{N1} is as defined herein.

In certain embodiments, the compound of Formula (I) is of the formula:

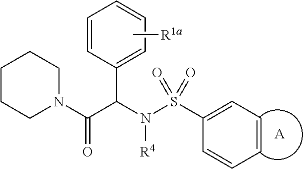

or a pharmaceutically acceptable salt thereof, wherein R^{N1} is as defined herein.

In certain embodiments, the compound of Formula (I) is of the formula:

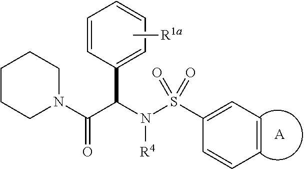

or a pharmaceutically acceptable salt thereof, wherein R^{N1} is as defined herein.

In certain embodiments, the compound of Formula (I) is of the formula:

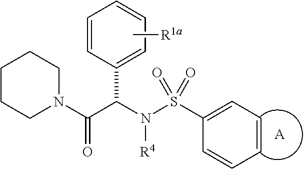

or a pharmaceutically acceptable salt thereof, wherein R^{N1} is as defined herein.

In certain embodiments, the compound of Formula (I) is of the following formula:

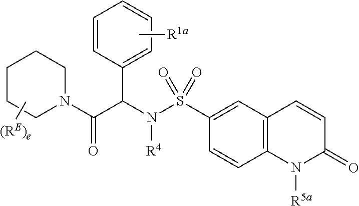

or a pharmaceutically acceptable salt thereof.

In certain embodiments, the compound of Formula (I) is of one of the following formulae:

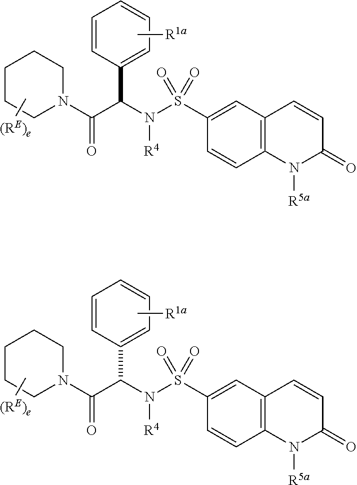

or a pharmaceutically acceptable salt thereof.

In certain embodiments, the compound of Formula (I) is of the following formula:

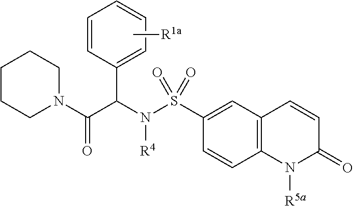

or a pharmaceutically acceptable salt thereof.

In certain embodiments, the compound of Formula (I) is of one of the following formulae:

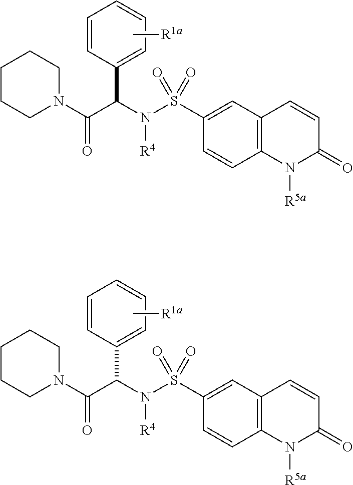

or a pharmaceutically acceptable salt thereof.

In certain embodiments, the compound of Formula (I) is of the following formula:

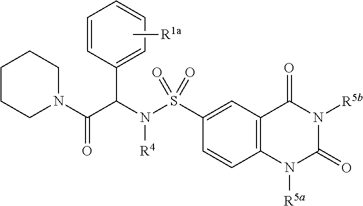

or a pharmaceutically acceptable salt thereof.

In certain embodiments, the compound of Formula (I) is of one of the following formulae:

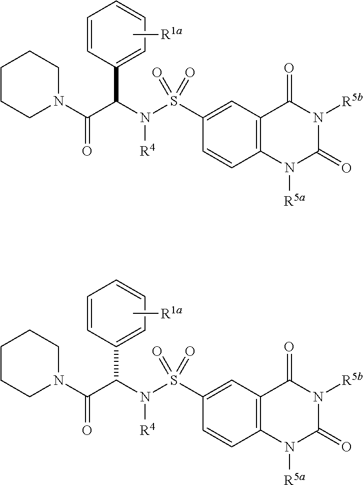

or a pharmaceutically acceptable salt thereof.

In certain embodiments, the compound of Formula (I) is of the formula:

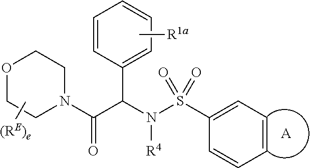

or a pharmaceutically acceptable salt thereof, wherein R^{N1} is as defined herein.

In certain embodiments, the compound of Formula (I) is of the formula:

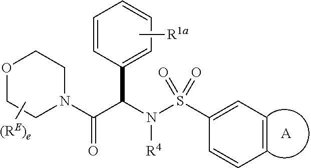

or a pharmaceutically acceptable salt thereof, wherein R^{N1} is as defined herein.

In certain embodiments, the compound of Formula (I) is of the formula:

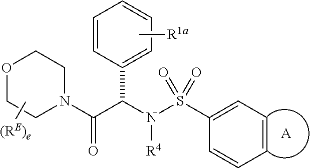

or a pharmaceutically acceptable salt thereof, wherein R^{N1} is as defined herein.

In certain embodiments, the compound of Formula (I) is of the formula:

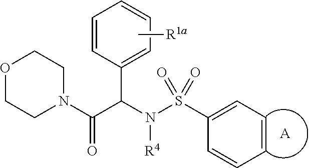

or a pharmaceutically acceptable salt thereof, wherein R^{N1} is as defined herein.

In certain embodiments, the compound of Formula (I) is of the formula:

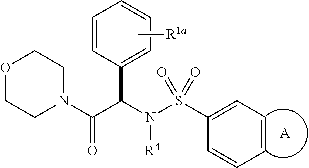

or a pharmaceutically acceptable salt thereof, wherein R^{N1} is as defined herein.

In certain embodiments, the compound of Formula (I) is of the formula:

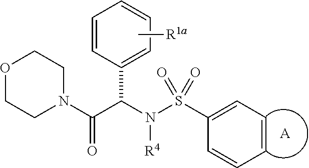

or a pharmaceutically acceptable salt thereof, wherein R^{N1} is as defined herein.

In certain embodiments, the compound of Formula (I) is of the following formula:

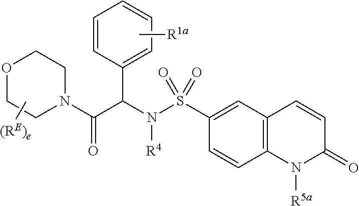

or a pharmaceutically acceptable salt thereof.

In certain embodiments, the compound of Formula (I) is of one of the following formulae:

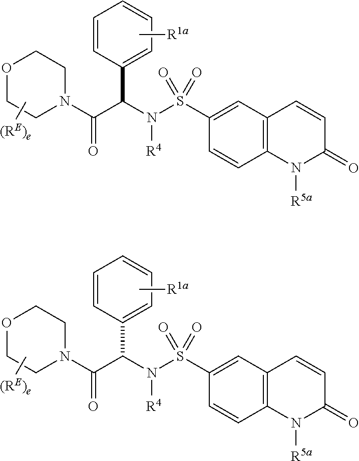

In certain embodiments, the compound of Formula (I) is of the following formula:

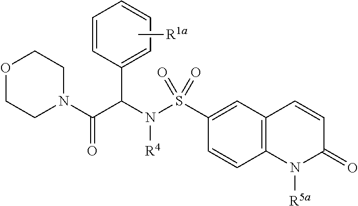

or a pharmaceutically acceptable salt thereof.

In certain embodiments, the compound of Formula (I) is of one of the following formulae:

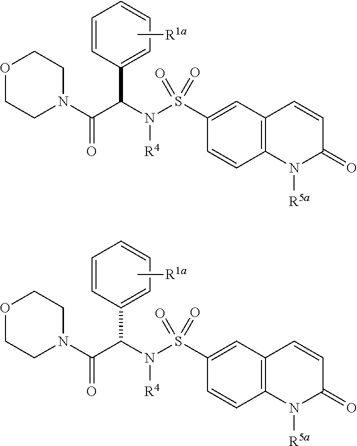

In certain embodiments, the compound of Formula (I) is of the following formula:

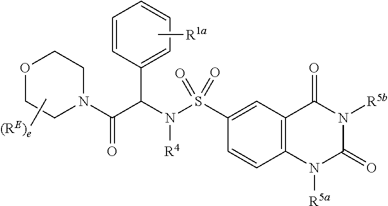

or a pharmaceutically acceptable salt thereof.

In certain embodiments, the compound of Formula (I) is of one of the following formulae:

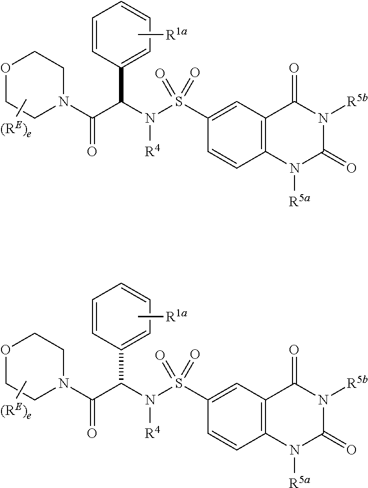

or a pharmaceutically acceptable salt thereof.

In certain embodiments, the compound of Formula (I) is of the following formula:

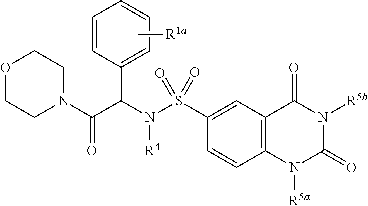

or a pharmaceutically acceptable salt thereof.

In certain embodiments, the compound of Formula (I) is of one of the following formulae:

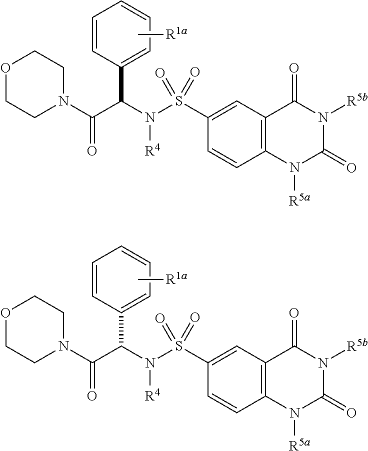

or a pharmaceutically acceptable salt thereof.

As generally defined herein, R⁴ is hydrogen, optionally substituted C₁₋₆ alkyl, or a nitrogen protecting group. In some embodiments, R⁴ is hydrogen. In some embodiments, R⁴ is optionally substituted C₁₋₆ alkyl. In some embodiments, R⁴ is unsubstituted C₁₋₆ alkyl. In some embodiments, R⁴ is substituted C₁₋₆ alkyl. In some embodiments, R⁴ is methyl or ethyl. In some embodiments, R⁴ is a nitrogen protecting group. In some embodiments, R⁴ is Bn, BOC, Cbz, or Fmoc.

In some embodiments, R¹ and R⁴ are taken together with the intervening nitrogen to form optionally substituted heteroaryl or optionally substituted heterocycle. In some embodiments, R¹ and R⁴ are taken together with the intervening nitrogen to form optionally substituted heteroaryl. In some embodiments, R¹ and R⁴ are taken together with the intervening nitrogen to form optionally substituted five-membered heteroaryl. In some embodiments, R¹ and R⁴ are taken together with the intervening nitrogen to form optionally substituted six-membered heteroaryl. In some embodiments, R¹ and R⁴ are taken together with the intervening nitrogen to form optionally substituted heterocycle. In some embodiments, R¹ and R⁴ are taken together with the intervening nitrogen to form optionally substituted five-membered heterocycle. In some embodiments, R¹ and R⁴ are taken together with the intervening nitrogen to form optionally substituted six-membered heterocycle. In some embodiments, R¹ and R⁴ are taken together with the intervening nitrogen to form optionally substituted 5,6-membered heterocycle. In some embodiments, R¹ and R⁴ are taken together with the intervening nitrogen to form optionally substituted isoindoline ring. In some embodiments, R¹ and R⁴ are taken together with the intervening nitrogen to form unsubstituted isoindoline ring. In some embodiments, R¹ and R⁴ are taken together with the intervening nitrogen to form optionally substituted 6,6-membered heterocycle. In some embodiments, R¹ and R⁴ are taken together with the intervening nitrogen to form optionally substituted dihydro-isoquinoline ring. In some embodiments, R¹ and R⁴ are taken together with the intervening nitrogen to form unsubstituted dihydro-isoquinoline ring. In some embodiments, R¹ and R⁴ are taken together with the intervening nitrogen to form a moiety of the formula

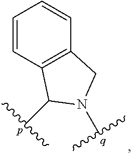 [SMILES: *C1c2ccccc2CN1*]

wherein p indicates point of attachment to

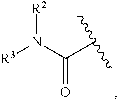

and q indicates point of attachment to

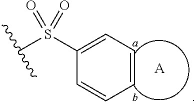

In some embodiments, R¹ and R⁴ are taken together with the intervening nitrogen to form a moiety of the formula

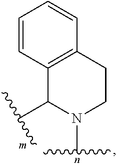 [SMILES: *C1c2ccccc2CCN1*]

wherein m
indicates point of attachment to

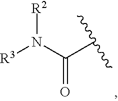

and n indicates point of attachment to

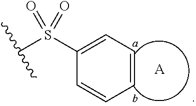

As generally defined herein, Ring A is of the formula

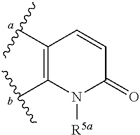

or formula

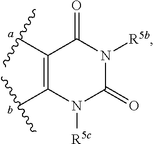

wherein a and b indicate points of attachment to the phenyl ring. In some embodiments, Ring A is of the formula

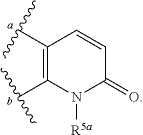

In some embodiments, Ring A is of the formula

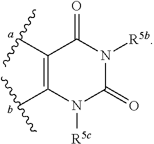

As generally defined herein, R^{5a} is hydrogen, optionally substituted C₁₋₆ alkyl, or a nitrogen protecting group. In some embodiments, R^{5a} is hydrogen. In some embodiments, R^{5a} is optionally substituted C₁₋₆ alkyl. In some embodiments, R^{5a} is unsubstituted C₁₋₆ alkyl. In some embodiments, R^{5a} is substituted C₁₋₆ alkyl. In some embodiments, R^{5a} is methyl or ethyl. In some embodiments, R^{5a} is a nitrogen protecting group. In some embodiments, R^{5a} is Bn, BOC, Cbz, or Fmoc.

As generally defined herein, R^{5b} is hydrogen, optionally substituted C₁₋₆ alkyl, or a nitrogen protecting group. In some embodiments, R^{5b} is hydrogen. In some embodiments, R^{5b} is optionally substituted C₁₋₆ alkyl. In some embodiments, R^{5b} is unsubstituted C₁₋₆ alkyl. In some embodiments, R^{5b} is substituted C₁₋₆ alkyl. In some embodiments, R^{5b} is methyl or ethyl. In some embodiments, R^{5b} is a nitrogen protecting group. In some embodiments, R^{5b} is Bn, BOC, Cbz, or Fmoc.

As generally defined herein, R^{5c} is hydrogen, optionally substituted C₁₋₆ alkyl, or a nitrogen protecting group. In some embodiments, R^{5c} is hydrogen. In some embodiments, R^{5c} is optionally substituted C₁₋₆ alkyl. In some embodiments, R^{5c} is unsubstituted C₁₋₆ alkyl. In some embodiments, R^{5c} is substituted C₁₋₆ alkyl. In some embodiments, R^{5c} is methyl or ethyl. In some embodiments, R^{5c} is a nitrogen protecting group. In some embodiments, R^{5c} is Bn, BOC, Cbz, or Fmoc.

In some embodiments, R^{5b} and R^{5c} are hydrogen. In some embodiments, R^{5b} is hydrogen and R^{5c} is optionally substituted C₁₋₆ alkyl. In some embodiments, R^{5c} is hydrogen and R^{5b} is optionally substituted C₁₋₆ alkyl.

In some embodiments, Ring A is of the formula

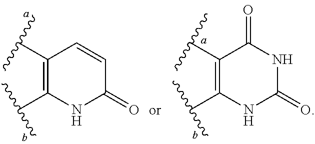 [SMILES: *c1ccc(=O)[nH]c1* ; *c1[nH]c(=O)[nH]c(=O)c1*]

### Synthesis of OGT Inhibitors

In some embodiments, compounds of the invention may be synthesized according to Scheme 1. Quinolin-2(1H)-one may be chlorosulfonylated using methods known to those skilled in the art for chlorosulfonylation, such as neat chlorosulfonic acid with the addition of heat. The resulting chlorosulfonylquinolinone may be reacted with an amino acid, for example, under aqueous basic conditions. A suitable aqueous base is, for example, aqueous sodium hydroxide. The amino acid may be a natural or unnatural amino acid. The resulting carboxylic acid may be further reacted with an amine under amide coupling conditions to furnish an amide. The amine may be a primary or secondary amine. Suitable coupling conditions are, for example, a coupling agent in the presence of base. A suitable coupling agent is, for example, HATU. A suitable base is, for example, Hunig's base.

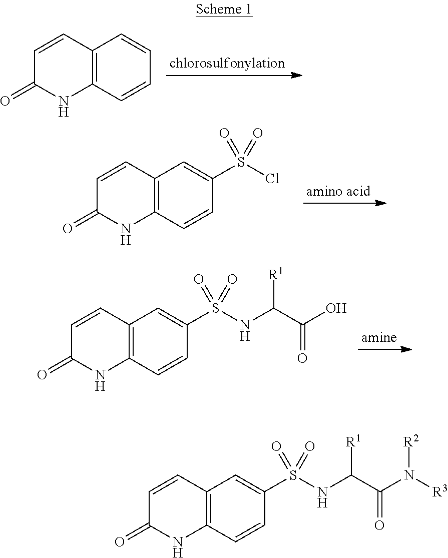

### Uses of OGT Inhibitors and Pharmaceutical Compositions Thereof

The invention further provides methods of treating a disease using a compound of the invention. The inventive method involves the administration of a therapeutically effective amount of an inventive compound to a subject (including, but not limited to, a human or other animal) in need thereof.

Compounds and compositions described herein are generally useful for the inhibition of the activity of O-GlcNAc transferase (OGT) or a variant or mutant thereof. OGT has been implicated in metabolic diseases such as diabetes and complications thereof, neurological diseases, proliferative diseases such as cancers, and autoimmune diseases, and inflammatory diseases (Golks, et al., EMBO Reports (2008) 9: 748-753; Liu, et al., Proc. Natl. Acad. Sci. USA (2004) 101: 10804-10809; Jones, Circulation Research (2005) 96: 925-926; Golks, et al., EMBO J. (2007) 26: 4369-4379; Ohn, et al., Nature Cell Biol. (2008) 10: 1224-1231),

The compounds and pharmaceutical compositions of the invention may be used in treating or preventing any disease or condition including, but not limited to, metabolic diseases (e.g., diabetes and complications thereof), proliferative diseases (e.g., cancers, benign neoplasms, diabetic retinopathy), neurodegenerative diseases, autoimmune diseases (e.g., rheumatoid arthritis, lupus, multiple sclerosis), and inflammatory diseases. The inventive compounds and pharmaceutical compositions may be administered to animals, preferably mammals (e.g., domesticated animals, cats, dogs, mice, rats), and more preferably humans. Any method of administration may be used to deliver the inventive compound or pharmaceutical composition to the animal. In certain embodiments, the compound or pharmaceutical composition is administered orally. In other embodiments, the compound or pharmaceutical composition is administered parenterally.

In certain embodiments, the invention provides methods for treating or lessening the severity of a metabolic disease. In certain embodiments, the invention provides methods for treating or lessening the severity of diabetes and complications thereof including, but not limited to, diabetes mellitus Type 1, diabetes melittus Type 2, insulin resistance, vascular disease, skin ulcers, circulatory damage, cardiac dysfunction, diabetic nephropathy, diabetic retinopathy, microvascular disease, macrovascular disease, and diabetic neuropathy. In certain embodiments, the invention provides methods for treating or lessening the severity of hyperglycemia, hyperinsulinemia, insulin resistance, or obesity.

In some embodiments, the invention provides methods for treating tumorogenesis.

In certain embodiments, the inventive compounds are useful in treating a proliferative disease. In some embodiments, the invention provides methods for treating cancer. Examples of cancers treated with compounds according to the invention include, but are not limited to, tumors of the breast; biliary tract; bladder; bone; brain, including glioblastomas and medulloblastomas; central and peripheral nervous system; cervix; colon; connective tissue; endocrine glands (e.g., thyroid and adrenal cortex); esophagus; endometrium; germ cells; gastrointestinal tract; head and neck; kidney; liver; lung; larynx and hypopharynx; mesothelioma; muscle; ovary, including those arising from epithelial cells, stromal cells, germ cells and mesenchymal cells; pancreas; prostate; rectum; renal, including adenocarcinoma and Wilms tumor; small intestine; soft tissue; testis, including germinal tumors such as seminoma, non-seminoma (teratomas, choriocarcinomas), stromal tumors, and germ cell tumors; thyroid, including thyroid adenocarcinoma and medullar carcinoma; stomach; skin, including melanoma, Kaposi's sarcoma, basocellular cancer, and squamous cell cancer; ureter; vagina; and vulva; retinoblastoma; leukemia and lymphoma, namely non-Hodgkins disease, lymphocytic lymphomas, chronic and acute myeloid leukemia (CML/AML), acute lymphoblastic leukemia (ALL), chronic lymphocytic leukemia (CLL), Hodgkins disease, multiple myeloma, and T-cell lymphoma; myelodysplastic syndrome; plasma cell neoplasia; paraneoplastic syndromes; intraepithelial neoplasms including Bowen's disease and Paget's disease; neuroblastomas; oral cancer including squamous cell carcinoma; sarcomas including leiomyosarcoma, rhabdomyosarcoma, liposarcoma, fibrosarcoma, and osteosarcoma; cancers of unknown primary site; and AIDS-related malignancies. Other cancers will be known to one of ordinary skill in the art.

In certain embodiments, the invention provides methods for treating or lessening the severity of autoimmune diseases including, but not limited to, inflammatory bowel disease, arthritis, systemic lupus erythematosus, rheumatoid arthritis, psoriatic arthritis, osteoarthritis, Still's disease, juvenile arthritis, diabetes, myasthenia gravis, Hashimoto's thyroiditis, Ord's thyroiditis, Graves' disease, Sjogren's syndrome, multiple sclerosis, Guillain-Barre syndrome, acute disseminated encephalomyelitis, Addison's disease, opsoclonus-myoclonus syndrome, ankylosing spondylosis, antiphospholipid antibody syndrome, aplastic anemia, autoimmune hepatitis, celiac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behcet's disease, chronic fatigue, dysautonomia, endometriosis, interstitial cystitis, neuromyotonia, scleroderma, or vulvodynia.

In some embodiments, the invention provides a method for treating or lessening the severity of one or more diseases and conditions, wherein the disease or condition is selected from immune-related conditions or diseases, which include, but are not limited to graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, and atopic dermatitis.

In some embodiments, the present invention provides a method for treating or lessening the severity of an inflammatory disease including, but not limited to, asthma, appendicitis, Blau syndrome, blepharitis, bronchiolitis, bronchitis, bursitis, cervicitis, cholangitis, cholecystitis, chronic obstructive pulmonary disease (COPD), chronic recurrent multifocal osteomyelitis (CRMO), colitis, conjunctivitis, cryopyrin associated periodic syndrome (CAPS), cystitis, dacryoadenitis, dermatitis, dermatomyositis, dry eye syndrome, encephalitis, endocarditis, endometritis, enteritis, enterocolitis, epicondylitis, epididymitis, familial cold-induced autoinflammatory syndrome, familial Mediterranean fever (FMF), fasciitis, fibrositis, gastritis, gastroenteritis, hepatitis, hidradenitis suppurativa, laryngitis, mastitis, meningitis, mevalonate kinase deficiency (MKD), Muckle-Well syndrome, myelitis myocarditis, myositis, nephritis, oophoritis, orchitis, osteitis, inflammatory osteolysis, otitis, pancreatitis, parotitis, pericarditis, peritonitis, pharyngitis, pleuritis, phlebitis, pneumonitis, pneumonia, proctitis, prostatitis, pulmonary fibrosis, pyelonephritis, pyoderma gangrenosum and acne syndrome (PAPA), pyogenic sterile arthritis, rhinitis, salpingitis, sinusitis, stomatitis, synovitis, systemic juvenile rheumatoid arthritis, tendonitis, TNF receptor associated periodic syndrome (TRAPS), tonsillitis, undifferentiated spondyloarthropathy, undifferentiated arthropathy, uveitis, vaginitis, vasculitis, vulvitis, chronic inflammation resulting from chronic viral or bacteria infections, or psoriasis (e.g., plaque psoriasis, pustular psoriasis, erythrodermic psoriasis, guttate psoriasis or inverse psoriasis).

In certain embodiments, the present invention provides methods for treating or lessening the severity of arthropathies and osteopathological diseases including, but not limited to, rheumatoid arthritis, osteoarthrtis, gout, polyarthritis, and psoriatic arthritis.

In certain embodiments, the present invention provides methods for treating or lessening the severity of acute and chronic inflammatory diseases including, but not limited to, ulcerative colitis, inflammatory bowel disease, Crohn's disease, dry eye syndrome, allergic rhinitis, allergic dermatitis, cystic fibrosis, chronic obstructive bronchitis, and asthma.

In certain embodiments, the invention provides methods for treating or lessening the severity of hyperproliferative diseases including, but not limited to, psoriasis or smooth muscle cell proliferation including vascular proliferative disorders, atherosclerosis, and restenosis. In certain embodiments, the invention provides methods for treating or lessening the severity of endometriosis, uterine fibroids, endometrial hyperplasia, and benign prostate hyperplasia.

In certain embodiments, the invention provides methods for treating or lessening the severity of neurodegenerative disorders and/or tauopathies including, but not limited to, Alzheimer's disease, progressive supranuclear palsy, corticobasal degeneration, frontotemporal lobar degeneration, Pick's disease, Parkinson's disease, Lewy body disease, or amyotropic lateral sclerosis (ALS).

The invention further includes a method for the treatment of mammals, including humans, which are suffering from one of the above-mentioned conditions, illnesses, disorders, or diseases. The method comprises that a therapeutically effective amount of one or more of the compounds according to this invention or a composition thereof is administered to the subject in need of such treatment.

The invention further includes a method for inhibiting OGT in a cell or tissue using a compound of the invention.

The invention further relates to the use of the inventive compounds for the production of pharmaceutical compositions which are employed for the treatment and/or prophylaxis and/or amelioration of the diseases, disorders, illnesses, and/or conditions as mentioned herein.

The invention further relates to the use of the inventive compounds for the production of pharmaceutical compositions that inhibit OGT.

The invention further relates to the use of the inventive compounds for the production of pharmaceutical compositions which can be used for treating, preventing, or ameliorating diseases responsive to inhibiting OGT, such as diabetes and complications thereof, neurodegenerative diseases, cancers, autoimmune diseases, and inflammatory diseases, such as any of those diseases mentioned herein.

The exact amount required will vary from subject to subject, depending on the species, age, and general condition of the subject, the particular compound, its mode of administration, its mode of activity, and the like. The compounds of the invention are preferably formulated in dosage unit form for ease of administration and uniformity of dosage. It will be understood, however, that the total daily usage of the proteins and compositions of the present invention will be decided by the attending physician within the scope of sound medical judgment. The specific therapeutically effective dose level for any particular patient or organism will depend upon a variety of factors including the disorder being treated and the severity of the disorder; the activity of the specific protein employed; the specific composition employed; the age, body weight, general health, sex, and diet of the patient; the time of administration, route of administration, and rate of excretion of the specific compound employed; the duration of the treatment; drugs used in combination or coincidental with the specific compound employed; and like factors well known in the medical arts.

Furthermore, after formulation with an appropriate pharmaceutically acceptable carrier in a desired dosage, the pharmaceutical compositions of this invention can be administered to humans and other animals orally, rectally, parenterally, intracisternally, intravaginally, intraperitoneally, topically (as by powders, ointments, or drops), bucally, as an oral or nasal spray, or the like. In certain embodiments, the compounds of the invention may be administered orally or parenterally at dosage levels sufficient to deliver from about 0.001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, and from about 1 mg/kg to about 25 mg/kg, of subject body weight per day, one or more times a day, to obtain the desired therapeutic effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations).

Liquid dosage forms for oral and parenteral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents. In certain embodiments for parenteral administration, the compounds of the invention are mixed with solubilizing agents such polyethoxylated castor oil, alcohols, oils, modified oils, glycols, polysorbates, cyclodextrins, polymers, and combinations thereof.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as poly(lactide-co-glycolide). Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active protein may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets, and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Formulations suitable for topical administration include liquid or semi-liquid preparations such as liniments, lotions, gels, applicants, oil-in-water or water-in-oil emulsions such as creams, ointments, or pastes; or solutions or suspensions such as drops. Formulations for topical administration to the skin surface can be prepared by dispersing the drug with a dermatologically acceptable carrier such as a lotion, cream, ointment, or soap. Useful carriers are capable of forming a film or layer over the skin to localize application and inhibit removal. For topical administration to internal tissue surfaces, the agent can be dispersed in a liquid tissue adhesive or other substance known to enhance adsorption to a tissue surface. For example, hydroxypropylcellulose or fibrinogen/thrombin solutions can be used to advantage. Alternatively, tissue-coating solutions such as pectin-containing formulations can be used. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Additionally, the carrier for a topical formulation can be in the form of a hydroalcoholic system (e.g., liquids and gels), an anhydrous oil or silicone based system, or an emulsion system, including, but not limited to, oil-in-water, water-in-oil, water-in-oil-in-water, and oil-in-water-in-silicone emulsions. The emulsions can cover a broad range of consistencies including thin lotions (which can also be suitable for spray or aerosol delivery), creamy lotions, light creams, heavy creams, and the like. The emulsions can also include microemulsion systems. Other suitable topical carriers include anhydrous solids and semisolids (such as gels and sticks); and aqueous based mousse systems.

It will also be appreciated that the compounds and pharmaceutical compositions of the present invention can be employed in combination therapies, that is, the compounds and pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another anticancer agent), or they may achieve different effects (e.g., control of any adverse effects).

In still another aspect, the present invention also provides a pharmaceutical pack or kit comprising one or more containers filled with one or more of the ingredients of the pharmaceutical compositions of the invention, and in certain embodiments, includes an additional approved therapeutic agent for use as a combination therapy. Optionally associated with such container(s) can be a notice in the form prescribed by a governmental agency regulating the manufacture, use or sale of pharmaceutical products, which notice reflects approval by the agency of manufacture, use or sale for human administration.

These and other aspects of the present invention will be further appreciated upon consideration of the following Examples, which are intended to illustrate certain particular embodiments of the invention but are not intended to limit its scope, as defined by the claims.

### EXAMPLES

### General Methods

### Reagents

UDP-¹⁴C-GlcNAc was purchased from Perkin Elmer. Streptavidin-HRP was purchased from Pierce. Immunoblotting reagents were purchased from Life Technologies. The OGA antibody (HPA036141) was purchased from Sigma. Anti-c-Myc antibody was purchased from Cell Signaling. RL2 (ab2739), and antibodies against Nup62, OGT (ab96718), Actin, H2B and H2B (Ser112GlcNAc) antibodies were purchased from Abcam. All biotynilated lectins were purchased from Vector Laboratories (BK-1000, BK-2000, BK-3000). D283 Med, CHO-K1, LNCaP, HEK and HeLa cells were obtained from ATCC. All cell culture reagents were purchased from Gibco. PUGNAC and Thiamet-G were purchased from Sigma Aldrich and Complete Protease inhibitor was purchased from Roche. The UDP-Glo assay was purchased from Promega. CKII3K peptide (KKKYPGGSTPVSSANMM) was purchased from Biomatik at 95% purity.

### General Chemistry

Unless otherwise stated, all reactions were carried out under an atmosphere of dry nitrogen in dried glassware. Indicated reaction temperatures refer to those of the reaction bath, while room temperature is noted as 23° C. All solvents were of anhydrous quality purchased from Sigma Chemical Co. (Saint Louis, Mo.) and were used as received. Commercially available starting materials and reagents were purchased from Sigma Chemical Co, Alfa Aesar Co, Acros Organics, or TCI America, and were used as received. (R)-2-((tert-butoxycarbonyl)amino)-2-(2-methoxyphenyl)acetic acid was purchased from Peptech Corporation (Bedford, Mass.) and was used as received.

Analytical thin layer chromatography (TLC) was performed with Sigma Aldrich TLC plates (5 cm×20 cm, 60 Å, 250 μm), and visualized by UV (254 nm) irradiation. Chromatography on silica gel was performed using forced flow (liquid) of the indicated solvent system on Biotage KP-Sil prepacked cartridges and using the Biotage SP-1 or the Biotage Isolera automated chromatography system. ¹H and ¹³C NMR spectra were recorded on an Inova 400 MHz spectrometer. Chemical shifts are reported in parts-per million (ppm) relative to tetramethylsilane. Spectra were referenced according to the solvent residual peak (CDCl₃ 7.26 ppm, 77.00 ppm, DMSO-d₆ 2.50 ppm, 39.50 ppm for ¹H, ¹³C, respectively). Data are reported as follows: chemical shift, multiplicity (s=singlet, d=doublet, t=triplet, q=quartet, br=broad, m=multiplet), coupling constants, and number of protons. Low-resolution mass spectra (electrospray ionization; ESI) were acquired on an Agilent Technologies 6130 quadrupole spectrometer coupled to the HPLC system. High-resolution mass spectral data were collected in-house using an Agilent 6210 time-of-flight mass spectrometer, also coupled to an Agilent Technologies 1200 series HPLC system. If needed, products were purified via a Waters semipreparative HPLC instrument equipped with a Phenomenex Luna C18 reverse phase (5 μm, 30 mm×75 mm) column having a flow rate of 45 mL/min. The mobile phase was a mixture of acetonitrile (0.025% TFA) and H₂O (0.05% TFA), and the temperature was maintained at 50° C.

Samples were analyzed for purity on an Agilent 1200 series LC/MS instrument equipped with a Luna C18 reverse phase (3 μm, 3 mm×75 mm) column having a flow rate of 0.8-1.0 mL/min over a 7 min gradient and a 8.5 min run time. Purity of final compounds was determined to be >95%, using a 3 μL injection with quantitation by AUC at 220 and 254 nm (Agilent diode array detector).

### Example 1

### Screening of a Library of Quinolinesulfonamides for OGT Inhibition

384-well plates (Costar #3654) were filled using a liquid handling robot with 20 μL of a mixture of 50 nM of a fluorescein-linked UDP-GlcNAc analog (see Gross et al, 2003), 1-2 μM sOGT, and buffer (20mM potassium phosphate, pH=7.4 with 500 μM tris(hydroxypropyl)phosphine). About 1000 compound library was serially diluted in DMSO from the 5 mg/ml plates fivefold 3 times, such that 4 different concentrations of compounds were prepared. Compound libraries of the 4 concentrations in duplicated were then transferred to the assay plates using a 100 nL pin array, resulting in a final compound concentration of 25 μg/mL or ˜70 μM at the highest of the four concentrations, assuming an average compound MW of 350. Using a Perkin Elmer Envision® microplate reader, the sample was excited at 480 nm in the vertical plane, and simultaneous emission intensity (535 nm) of the vertical and horizontal polarization planes was measured. The polarization was calculated using the following equation:eq4: mP=1000*(V−G*H)/(V+G*H)where: mP=millipolarization units, V=intensity of vertically polarized emission (RFU), H=intensity of horizontally polarized emission (RFU), and G=gain. Compounds were evaluated for their ability to affect the fluorescent polarization of the probe and the results are shown in Table 1.

TABLE 1
OGT Inhibition Data

| | % OGT |
|---|---|
| | activity |
| | (100 μM |
| Structure | treatment) |
| 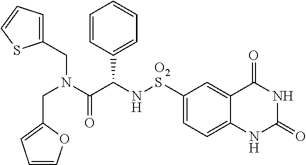 [SMILES: O=C([C@@H](NS(=O)(=O)c1ccc2[nH]c(=O)[nH]c(=O)c2c1)c1ccccc1)N(Cc1ccco1)Cc1cccs1] | No inhibition |
| 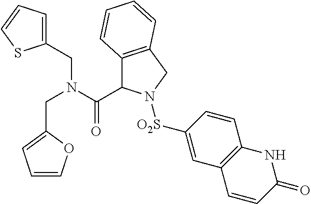 [SMILES: O=C(C1c2ccccc2CN1S(=O)(=O)c1ccc2[nH]c(=O)ccc2c1)N(Cc1ccco1)Cc1cccs1] | 56% |
| 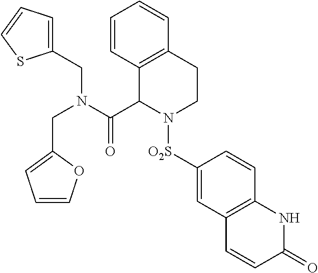 [SMILES: O=C(C1c2ccccc2CCN1S(=O)(=O)c1ccc2[nH]c(=O)ccc2c1)N(Cc1ccco1)Cc1cccs1] | 47% |
| 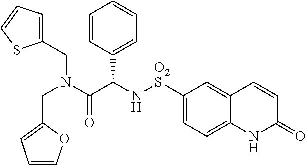 [SMILES: O=C([C@@H](NS(=O)(=O)c1ccc2[nH]c(=O)ccc2c1)c1ccccc1)N(Cc1ccco1)Cc1cccs1] | 7% |
| 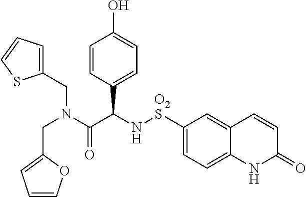 [SMILES: O=C([C@H](NS(=O)(=O)c1ccc2[nH]c(=O)ccc2c1)c1ccc(O)cc1)N(Cc1ccco1)Cc1cccs1] | 0.7% |
| 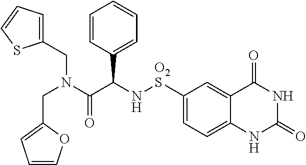 [SMILES: O=C([C@H](NS(=O)(=O)c1ccc2[nH]c(=O)[nH]c(=O)c2c1)c1ccccc1)N(Cc1ccco1)Cc1cccs1] | 68% |
| 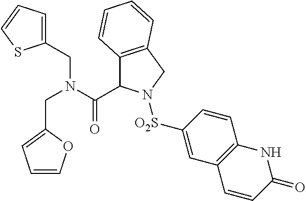 [SMILES: O=C(C1c2ccccc2CN1S(=O)(=O)c1ccc2[nH]c(=O)ccc2c1)N(Cc1ccco1)Cc1cccs1] | 43% |
| 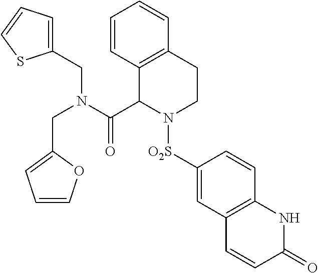 [SMILES: O=C(C1c2ccccc2CCN1S(=O)(=O)c1ccc2[nH]c(=O)ccc2c1)N(Cc1ccco1)Cc1cccs1] | 65% |
| 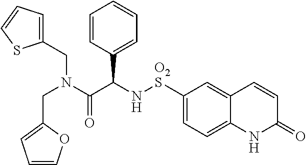 [SMILES: O=C([C@H](NS(=O)(=O)c1ccc2[nH]c(=O)ccc2c1)c1ccccc1)N(Cc1ccco1)Cc1cccs1] | 1.73% |
| 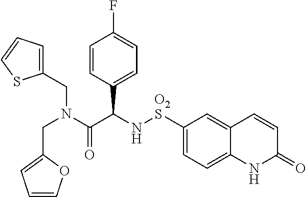 [SMILES: O=C([C@H](NS(=O)(=O)c1ccc2[nH]c(=O)ccc2c1)c1ccc(F)cc1)N(Cc1ccco1)Cc1cccs1] | 5% |
| 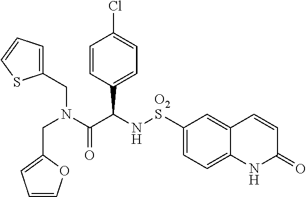 [SMILES: O=C([C@H](NS(=O)(=O)c1ccc2[nH]c(=O)ccc2c1)c1ccc(Cl)cc1)N(Cc1ccco1)Cc1cccs1] | 22% |
| 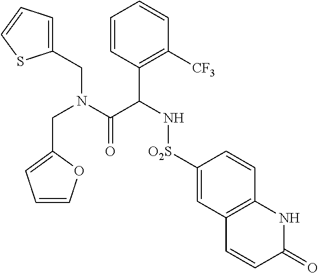 [SMILES: O=C(C(NS(=O)(=O)c1ccc2[nH]c(=O)ccc2c1)c1ccccc1C(F)(F)F)N(Cc1ccco1)Cc1cccs1] | 0.7% |
| 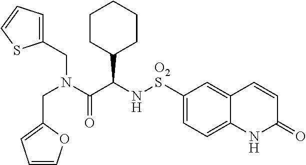 [SMILES: O=C([C@H](NS(=O)(=O)c1ccc2[nH]c(=O)ccc2c1)C1CCCCC1)N(Cc1ccco1)Cc1cccs1] | 22% |
| 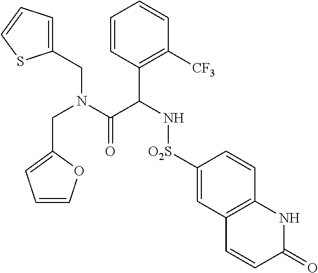 [SMILES: O=C(C(NS(=O)(=O)c1ccc2[nH]c(=O)ccc2c1)c1ccccc1C(F)(F)F)N(Cc1ccco1)Cc1cccs1] | 11% |
| 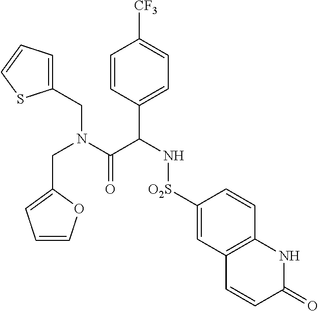 [SMILES: O=C(C(NS(=O)(=O)c1ccc2[nH]c(=O)ccc2c1)c1ccc(C(F)(F)F)cc1)N(Cc1ccco1)Cc1cccs1] | 33% |
| 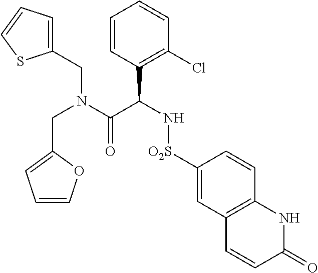 [SMILES: O=C([C@H](NS(=O)(=O)c1ccc2[nH]c(=O)ccc2c1)c1ccccc1Cl)N(Cc1ccco1)Cc1cccs1] | 4% |
| 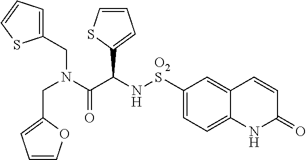 [SMILES: O=C([C@H](NS(=O)(=O)c1ccc2[nH]c(=O)ccc2c1)c1cccs1)N(Cc1ccco1)Cc1cccs1] | 7% |
| 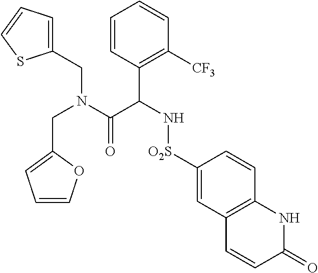 [SMILES: O=C(C(NS(=O)(=O)c1ccc2[nH]c(=O)ccc2c1)c1ccccc1C(F)(F)F)N(Cc1ccco1)Cc1cccs1] | 38% |
| 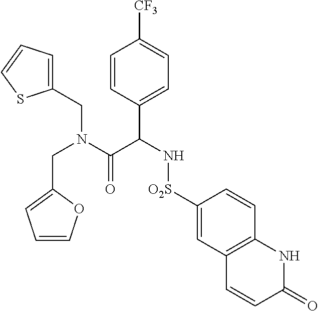 [SMILES: O=C(C(NS(=O)(=O)c1ccc2[nH]c(=O)ccc2c1)c1ccc(C(F)(F)F)cc1)N(Cc1ccco1)Cc1cccs1] | 54% |
| 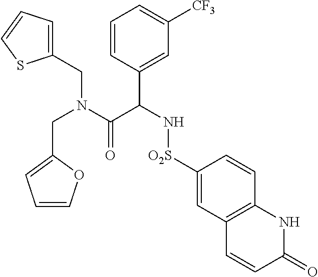 [SMILES: O=C(C(NS(=O)(=O)c1ccc2[nH]c(=O)ccc2c1)c1cccc(C(F)(F)F)c1)N(Cc1ccco1)Cc1cccs1] | 29% |
| 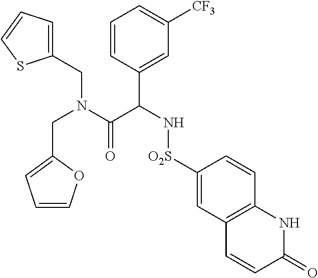 [SMILES: O=C(C(NS(=O)(=O)c1ccc2[nH]c(=O)ccc2c1)c1cccc(C(F)(F)F)c1)N(Cc1ccco1)Cc1cccs1] | 7.4% |
| 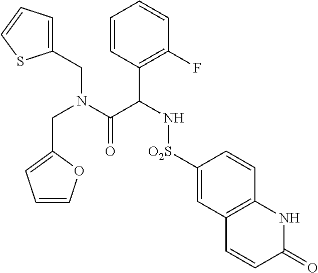 [SMILES: O=C(C(NS(=O)(=O)c1ccc2[nH]c(=O)ccc2c1)c1ccccc1F)N(Cc1ccco1)Cc1cccs1] | 0.5% |
| 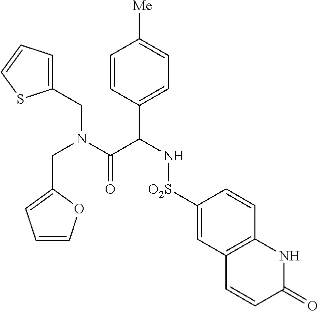 [SMILES: Cc1ccc(C(NS(=O)(=O)c2ccc3[nH]c(=O)ccc3c2)C(=O)N(Cc2ccco2)Cc2cccs2)cc1] | 11% |
| 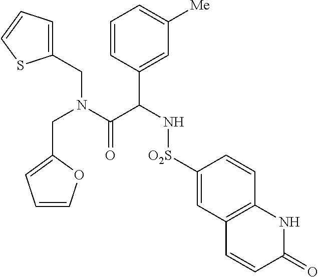 [SMILES: Cc1cccc(C(NS(=O)(=O)c2ccc3[nH]c(=O)ccc3c2)C(=O)N(Cc2ccco2)Cc2cccs2)c1] | No inhibition |
| 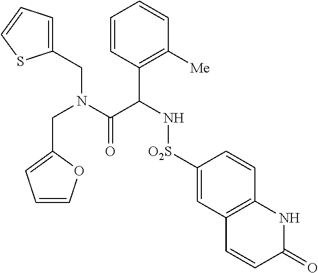 [SMILES: Cc1ccccc1C(NS(=O)(=O)c1ccc2[nH]c(=O)ccc2c1)C(=O)N(Cc1ccco1)Cc1cccs1] | 4.5% |
| 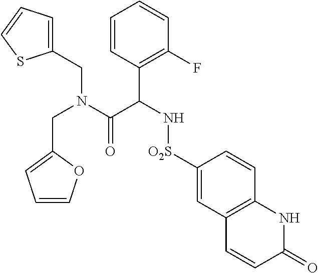 [SMILES: O=C(C(NS(=O)(=O)c1ccc2[nH]c(=O)ccc2c1)c1ccccc1F)N(Cc1ccco1)Cc1cccs1] | 24% |
| 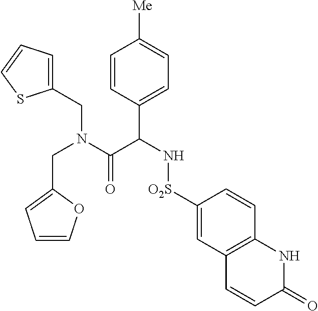 [SMILES: Cc1ccc(C(NS(=O)(=O)c2ccc3[nH]c(=O)ccc3c2)C(=O)N(Cc2ccco2)Cc2cccs2)cc1] | 66% |
| 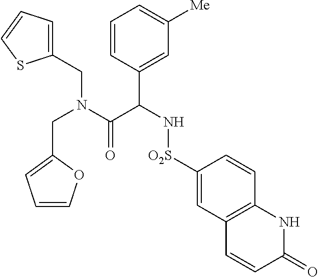 [SMILES: Cc1cccc(C(NS(=O)(=O)c2ccc3[nH]c(=O)ccc3c2)C(=O)N(Cc2ccco2)Cc2cccs2)c1] | No inhibition |
| 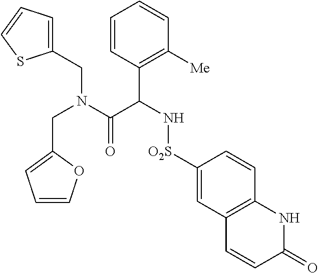 [SMILES: Cc1ccccc1C(NS(=O)(=O)c1ccc2[nH]c(=O)ccc2c1)C(=O)N(Cc1ccco1)Cc1cccs1] | 10% |
| 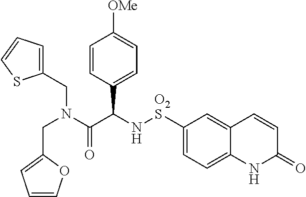 [SMILES: COc1ccc([C@@H](NS(=O)(=O)c2ccc3[nH]c(=O)ccc3c2)C(=O)N(Cc2ccco2)Cc2cccs2)cc1] | 2.9% |
| 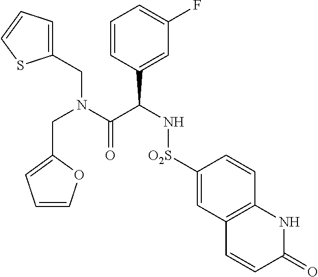 [SMILES: O=C([C@H](NS(=O)(=O)c1ccc2[nH]c(=O)ccc2c1)c1cccc(F)c1)N(Cc1ccco1)Cc1cccs1] | 2.6% |
| 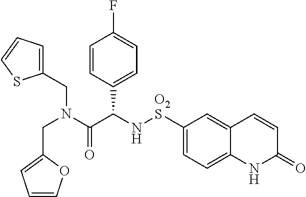 [SMILES: O=C([C@@H](NS(=O)(=O)c1ccc2[nH]c(=O)ccc2c1)c1ccc(F)cc1)N(Cc1ccco1)Cc1cccs1] | 0.9% |
| 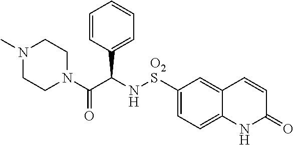 [SMILES: CN1CCN(C(=O)[C@H](NS(=O)(=O)c2ccc3[nH]c(=O)ccc3c2)c2ccccc2)CC1] | 47% |
| 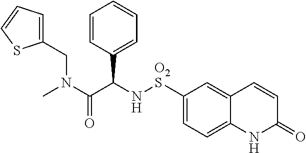 [SMILES: CN(Cc1cccs1)C(=O)[C@H](NS(=O)(=O)c1ccc2[nH]c(=O)ccc2c1)c1ccccc1] | 2.5% |
| 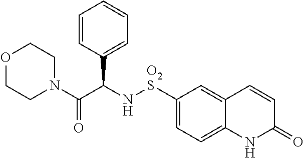 [SMILES: O=C([C@H](NS(=O)(=O)c1ccc2[nH]c(=O)ccc2c1)c1ccccc1)N1CCOCC1] | 47% |
| 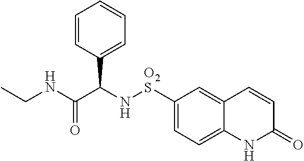 [SMILES: CCNC(=O)[C@H](NS(=O)(=O)c1ccc2[nH]c(=O)ccc2c1)c1ccccc1] | 26% |
| 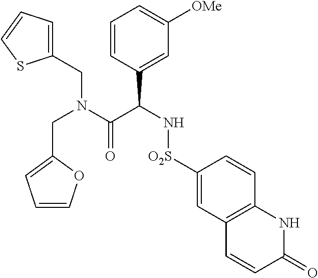 [SMILES: COc1cccc([C@@H](NS(=O)(=O)c2ccc3[nH]c(=O)ccc3c2)C(=O)N(Cc2ccco2)Cc2cccs2)c1] | 2.5% |
| 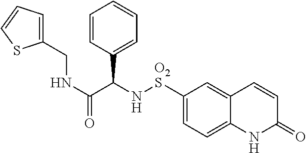 [SMILES: O=C(NCc1cccs1)[C@H](NS(=O)(=O)c1ccc2[nH]c(=O)ccc2c1)c1ccccc1] | No inhibition |
| 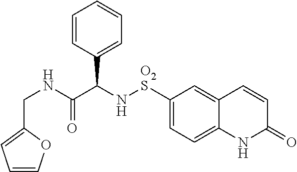 [SMILES: O=C(NCc1ccco1)[C@H](NS(=O)(=O)c1ccc2[nH]c(=O)ccc2c1)c1ccccc1] | 64% |
| 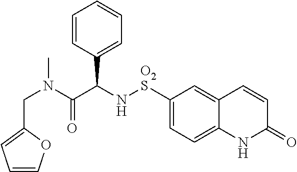 [SMILES: CN(Cc1ccco1)C(=O)[C@H](NS(=O)(=O)c1ccc2[nH]c(=O)ccc2c1)c1ccccc1] | 5% |
| 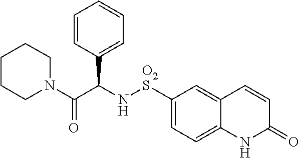 [SMILES: O=C([C@H](NS(=O)(=O)c1ccc2[nH]c(=O)ccc2c1)c1ccccc1)N1CCCCC1] | 22% |
| 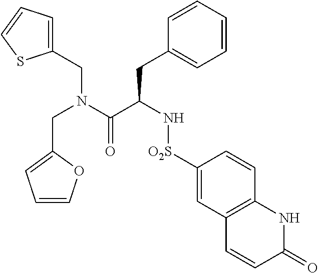 [SMILES: O=C([C@@H](Cc1ccccc1)NS(=O)(=O)c1ccc2[nH]c(=O)ccc2c1)N(Cc1ccco1)Cc1cccs1] | 53% |
| 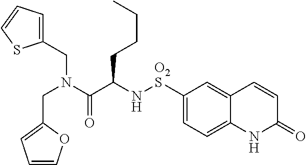 [SMILES: CCCC[C@@H](NS(=O)(=O)c1ccc2[nH]c(=O)ccc2c1)C(=O)N(Cc1ccco1)Cc1cccs1] | 8% |

TABLE 1A
sOGT Inhibition by Additional Exemplified Compounds

| | IC50 |
|---|---|
| Structure | (μM) |
| 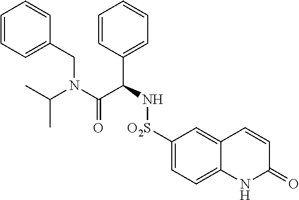 [SMILES: CC(C)N(Cc1ccccc1)C(=O)[C@H](NS(=O)(=O)c1ccc2[nH]c(=O)ccc2c1)c1ccccc1] | 7 |
| 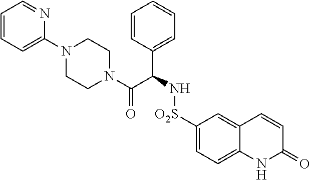 [SMILES: O=C([C@H](NS(=O)(=O)c1ccc2[nH]c(=O)ccc2c1)c1ccccc1)N1CCN(c2ccccn2)CC1] | 10 |
| 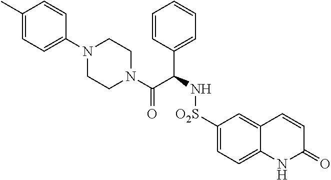 [SMILES: Cc1ccc(N2CCN(C(=O)[C@H](NS(=O)(=O)c3ccc4[nH]c(=O)ccc4c3)c3ccccc3)CC2)cc1] | 9 |
| 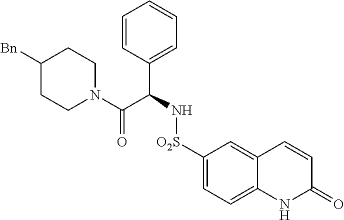 [SMILES: O=C([C@H](NS(=O)(=O)c1ccc2[nH]c(=O)ccc2c1)c1ccccc1)N1CCC(Cc2ccccc2)CC1] | Ki = 2 |

*Data were collected from the fluorescence displacement in vitro assay via stated above.

### Example 2

A Small Molecule that Inhibits OGT Activity in Cells

In order to validate OGT as a therapeutic target and gain a deeper understanding of its primary biological functions, small molecule OGT inhibitors that demonstrate selective, on-target inhibition in cells are required^{17,18}. While various small molecules are reported to perturb O-GlcNAc in cells (Table 2), including alloxan, a uracil mimic, and benzyl 2-acetamido-2-deoxy-α-D-galactopyranoside (BAGDP), a N-acetylgalactosamine (GalNAc) mimic, most of these compounds have not been shown to inhibit OGT selectively in cells. Indeed, many reports do not demonstrate OGT inhibition, but rather rely on cellular viability or other downstream readouts as a proxy. In the case of alloxan, it has even been shown that its ability to inhibit OGA surpasses its ability to inhibit OGT^{19,20}, while BAGDP likely inhibits numerous carbohydrate processing enzymes²¹. Some substrate and bisubstrate mimics that inhibit OGT in vitro have been reported, but these inhibitors are not membrane permeable and hence are ineffective in cells²²⁻²⁵. However, in the case of one substrate mimetic inhibitor, UDP-5SGlcNAc, the lack of cell-permeability was overcome by feeding cells a metabolic precursor, Ac4-5SGlcNAc, which is converted to UDP-5SGlcNAc in vivo²⁵. Ac4-5SGlcNAc dramatically reduces global O-GlcNAcylation in cells, in part because the active form of the inhibitor, UDP-5SGlcNAc accumulates in cells²⁶. As an isostere of UDP-GlcNAc, UDP-5SGlcNAc may inhibit not only OGT, but also other UDP-GlcNAc-dependent enzymes²⁵. Moreover, UDP-5SGlcNAc is epimerized to UDP-5SGalNAc in cells and enzymes that use UDP-GalNAc may also be affected. While Ac4-5SGlcNAc is currently the best cellular inhibitor of OGT, these caveats must be considered when using it. Furthermore, prospects for overcoming off-target effects are limited for close substrate analogs, particularly if they require enzymatic processing in order to become active. Thus, there remains a pressing need for cell-permeable small molecule OGT inhibitors that are amenable to chemical modification.

TABLE 2
Small-molecules used for the pharmacological inhibition of OGT in cells

| | Concentration used |
|---|---|
| | for cellular work and |
| Compound | respective citation |
| 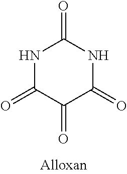 | 5 mM⁴⁷, 5 mM48, 5 mM⁴⁹, 5 mM⁵⁰, 5 mM⁵¹ 5 mM⁵², unknown⁵³, 2.5 mM⁵⁴ |
| 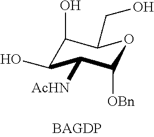 | 2 mM⁵⁵, 2.5 mM⁵⁶, 1 mM⁵⁷ |
| 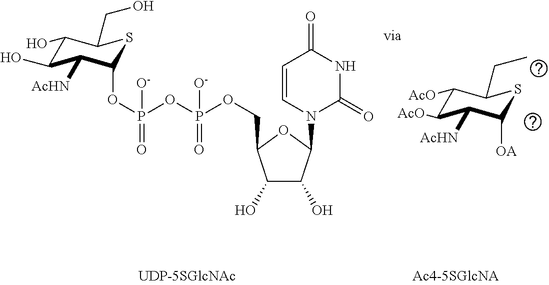 | 100 μM²⁶, various²⁵, 100 μM⁵⁸ |
| 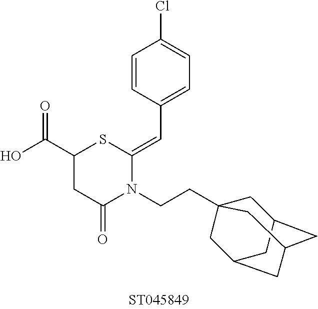 [SMILES: O=C(O)C1CC(=O)N(CCC23CC4CC(CC(C4)C2)C3)/C(=C/c2ccc(Cl)cc2)S1] | 100 μM⁵⁹, 10-100 μM¹⁵, 20 μM⁶⁰ 100 μM⁶¹, unknown⁶², 50 μM⁶³ |
| 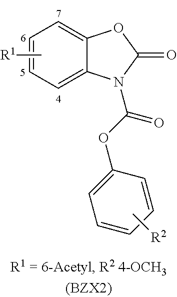 | 200 μM³³ |
| 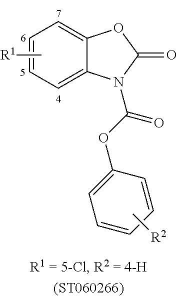 | 50-100 μM¹⁰, 100 μM³², 500 μM¹⁶ 500 μM⁵¹, 50 μM⁶⁴, 50 μM⁶⁵ |

A survey of the literature illustrating the use of various small-molecules claimed to inhibit OGT in cells. Only Ac4-5SGlcNAc
(UDP-5SGlcNAc) explicitly demonstrated inhibition of a known OGT target (nup62) in cells²⁵.

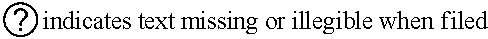

High-throughput screening (HTS) approaches have been adopted to identify small molecule inhibitor scaffolds for glycosyltransferases, including OGT²⁷⁻³⁰ (see Example 1). Several OGT inhibitors of modest potency were identified in a polarization-based fluorescence displacement screen, and three such compounds were reported as in vitro inhibitors²⁷. Although robust inhibition of OGT in cells was not shown for these compounds³¹, they nonetheless came into use as OGT inhibitors for biological studies. One compound was shown to operate by an interesting covalent mechanism of action, but proved too reactive to use as a selective OGT inhibitor in cells^{10, 16, 32, 33}. Therefore, the screening data were reexamined to identify scaffolds amenable to optimization and a quinolinone-6-sulfonamide (Q6S) class of compounds appeared promising. A cell-permeable OGT inhibitor has been identified from a biased library screen followed by analog synthesis. The inhibitor shows on-target OGT engagement, as judged by several readouts, but does not appear to alter N- or O-glycan structures substantially. This work validates the utility of a HTS approach for identifying scaffolds that can lead to probe molecules for studies of OGT's cellular roles.

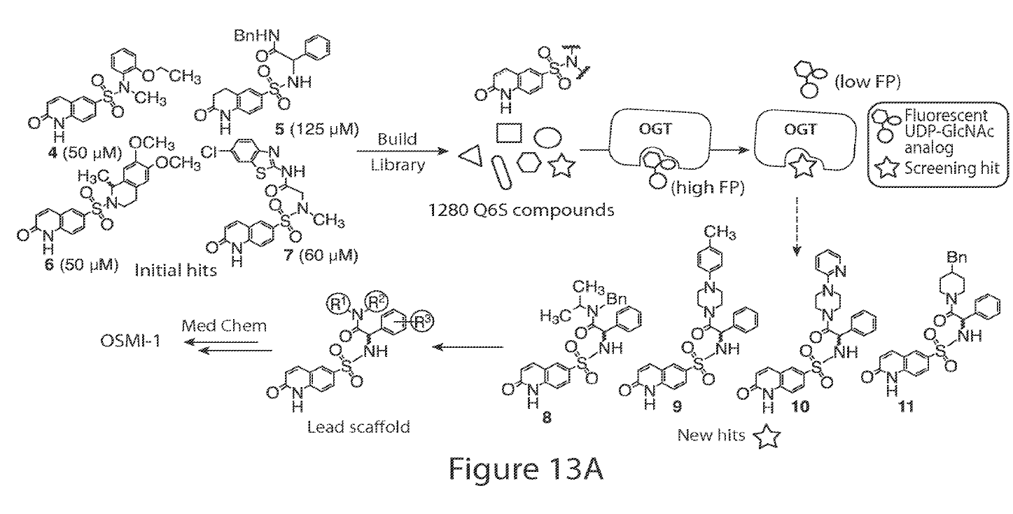

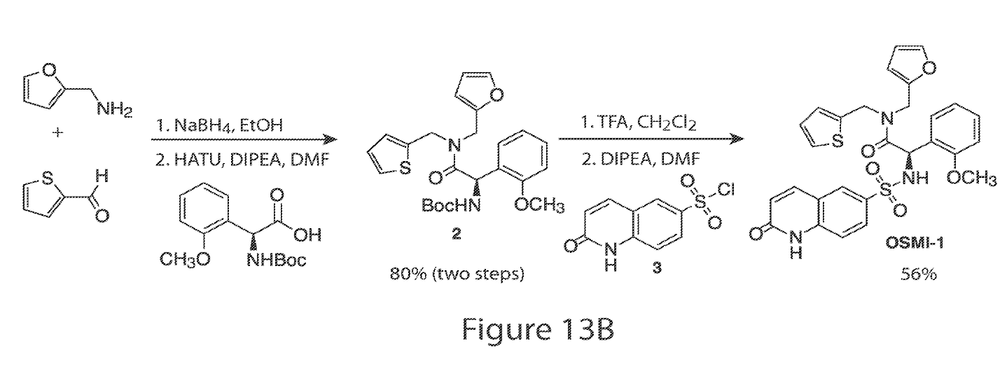

Four previously undisclosed compounds from our HTS (FIG. 13, top left)³¹ were found to contain either a Q6S or a 3,4-dihydro-quinoline-6-sulfonamide core (henceforth both will be referred to as Q6S; see FIG. 13A). Related compounds with substitutions in the Q6S core were not hits in the screen. While the potency of the compounds was weak, the conserved core was deemed promising and so a library of 1,280 commercial compounds bearing a Q6S moiety was assembled. Compounds were screened at five different concentrations in a fluorescence displacement assay (FIG. 13A)²⁷. Using the known binding constant for the substrate analog, the dose-response data allowed us to estimate binding affinities (Kᵢ values) for the hits. The top 40 hits from the primary screen, ranked by Kᵢ, were tested in a secondary radiometric capture assay. The top four confirmed hits all bore a phenylglycine (compounds 8-11 in FIG. 13A), the Q6S core, and an amide. Various analogs were prepared based on a modular assembly (as in FIG. 13B), and OGT inhibition was assessed both in vitro and in cells. These efforts, which will be described in details elsewhere, resulted in the identification of an analog, OSMI-1 (FIG. 13B).

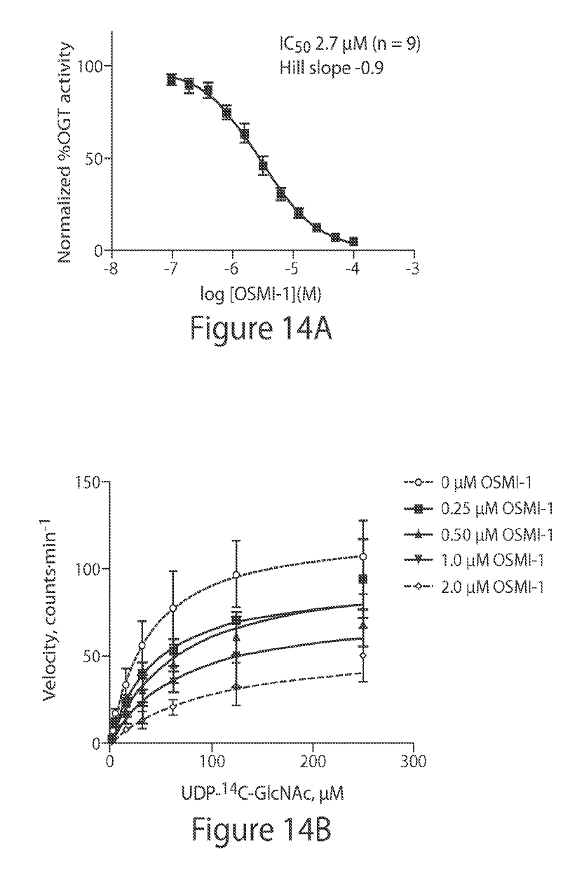

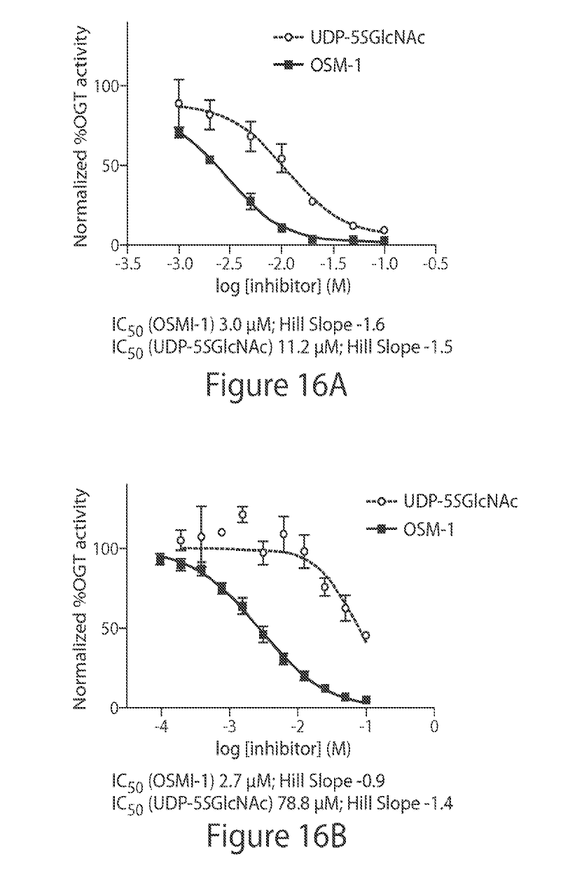

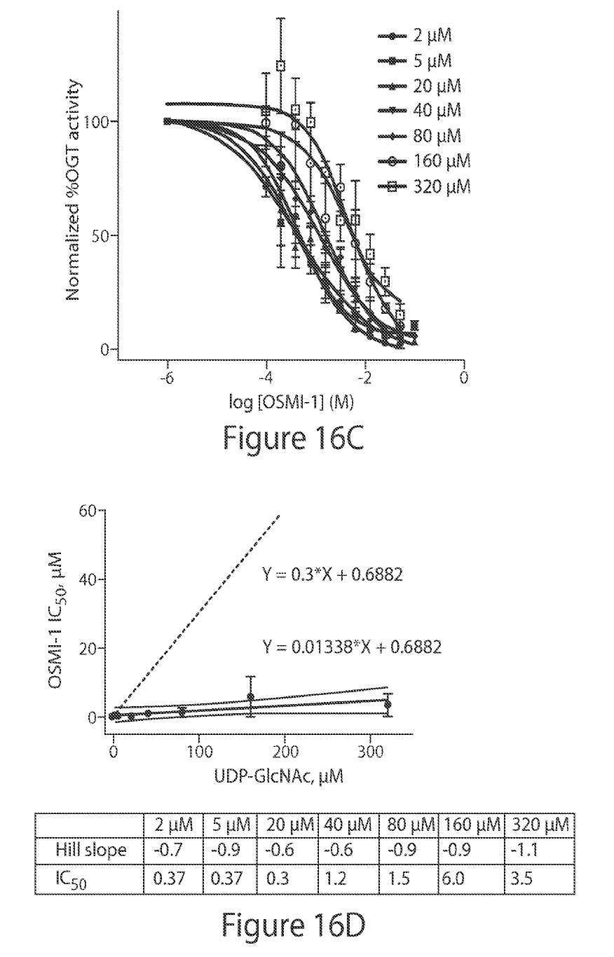

Compound OSMI-1 was tested to inhibit full length human OGT (ncOGT) in a coupled enzyme assay that measures the UDP produced when GlcNAc is transferred from UDP-GlcNAc to a peptide acceptor. OSMI-1 inhibited ncOGT with an IC₅₀ value of 2.7 μM (FIG. 14A). A similar IC₅₀ value was obtained using a radiometric capture assay in which a well-characterized protein substrate, nucleoporin62 (Nup62), a heavily glycosylated component of the nuclear pore, is the acceptor substrate (FIG. 16A)³⁴. In contrast, the IC₅₀ values obtained for UDP-5SGlcNAc in these two assays were 78.8 and 11.1 μM, respectively. The UDP-GlcNAc concentrations used in the two assays differed by about 6-fold, and given that UDP-5SGlcNAc is a competitive inhibitor with respect to UDP-GlcNAc²⁵, this shift in IC₅₀ value was expected (FIG. 16 and Equation 1). Since the IC₅₀ for OSMI-1 was largely insensitive to UDP-GlcNAc concentration (FIGS. 16C and 16D), we concluded that it did not act competitively with respect to the donor sugar substrate. Consistent with this, it was observed that the Vₘₐₓ for glycosylation, under saturating acceptor and variable UDP-GlcNAc conditions, decreased with increasing OSMI-1 concentration (FIG. 14B).

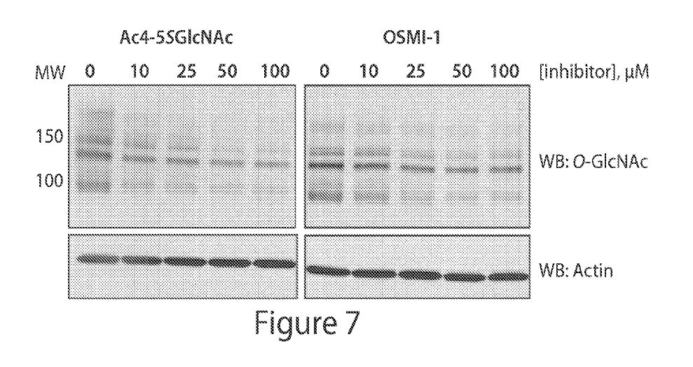

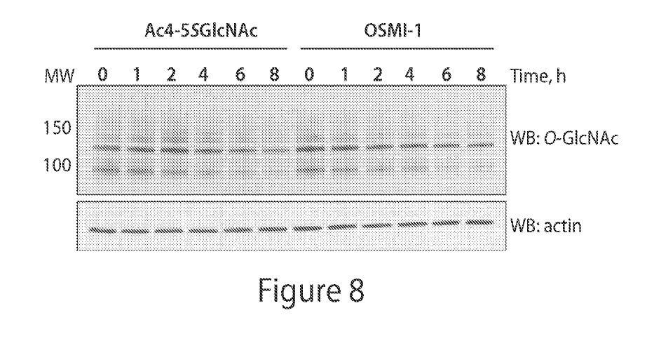

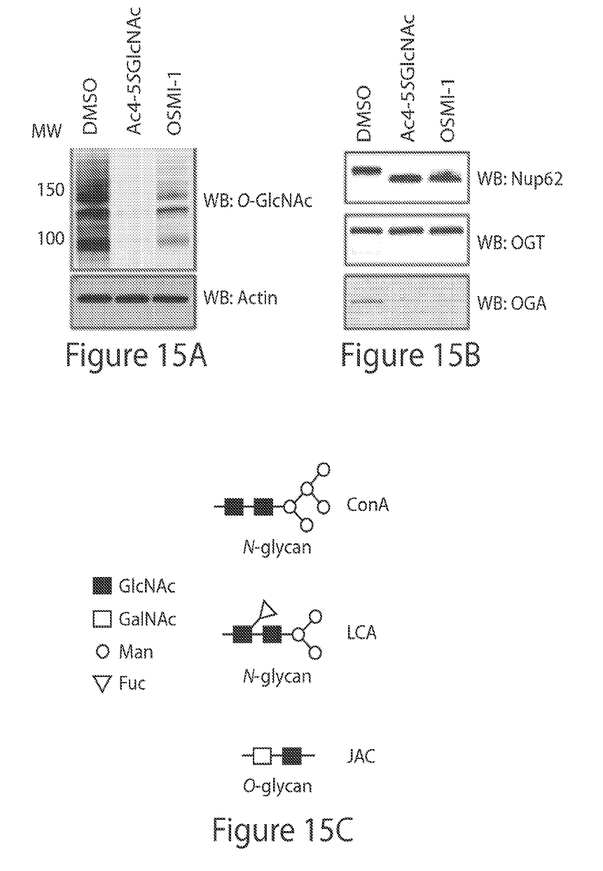

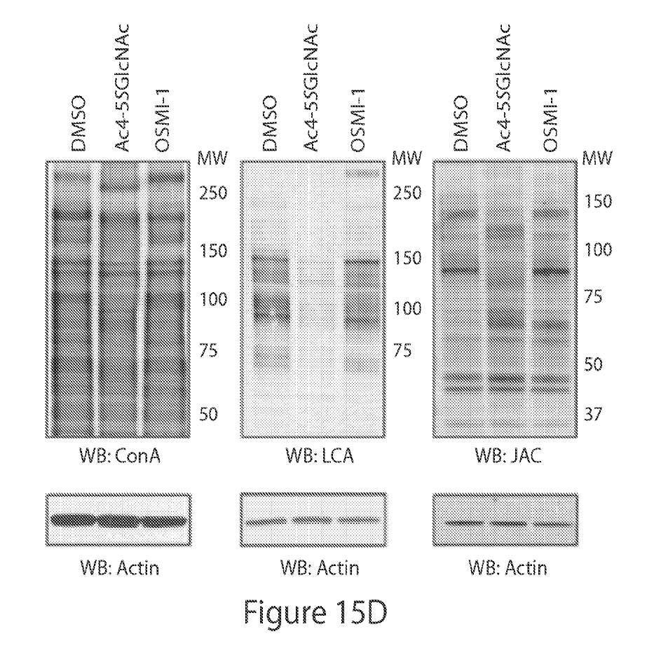

We next examined the ability of OSMI-1 to inhibit global O-GlcNAcylation in Chinese hamster ovary (CHO) cells. This cell line was used previously to evaluate Ac4-5SGlcNAc as an inhibitor²⁵. Cells were treated for 24 hours with varying concentrations of OSMI-1 ranging from 10-100 μM and cell lysates were probed with the O-GlcNAc antibody RL2³⁵. OSMI-1 reduced global O-GlcNAcylation (FIG. 15A) in a dose-dependent manner (FIG. 7), with the maximal effect being achieved at 50 μM. Due to the limited aqueous solubility of OSMI-1, higher concentrations of OSMI-1 did not further reduce O-GlcNAc levels (FIG. 8). When used at 50 μM, Ac4-5SGlcNAc reduced global O-GlcNAcylation to a greater extent than OSMI-1 even though it is a less potent inhibitor in vitro. UDP-5SGlcNAc reaches substantially higher intracellular concentrations than OSMI-1 because it cannot diffuse freely across the cell membrane; once formed from Ac4-5SGlcNAc, it accumulates intracellularly, allowing it to compete successfully with UDP-GlcNAc²⁵. Although OSMI-1 is cell permeable, it is relatively large and likely does not reach cellular concentrations comparable with the administered dose.

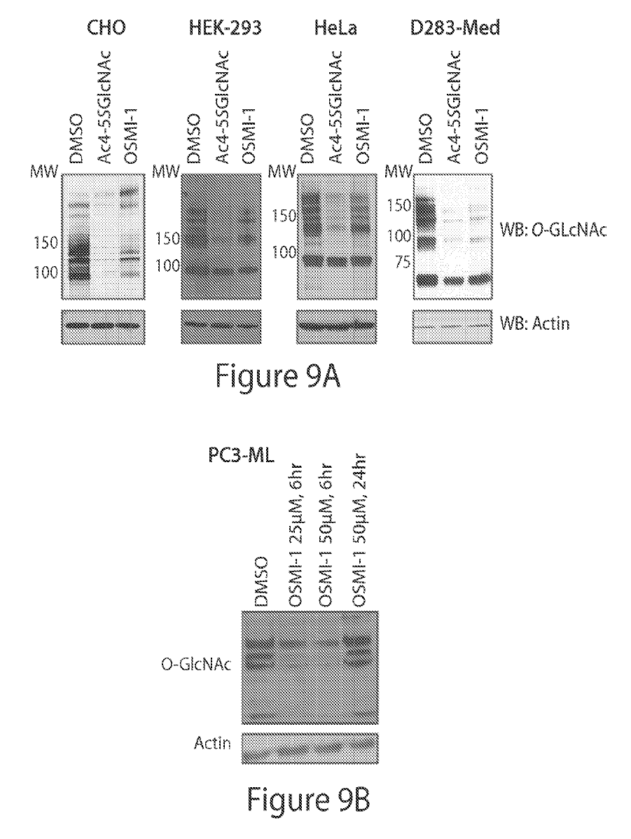

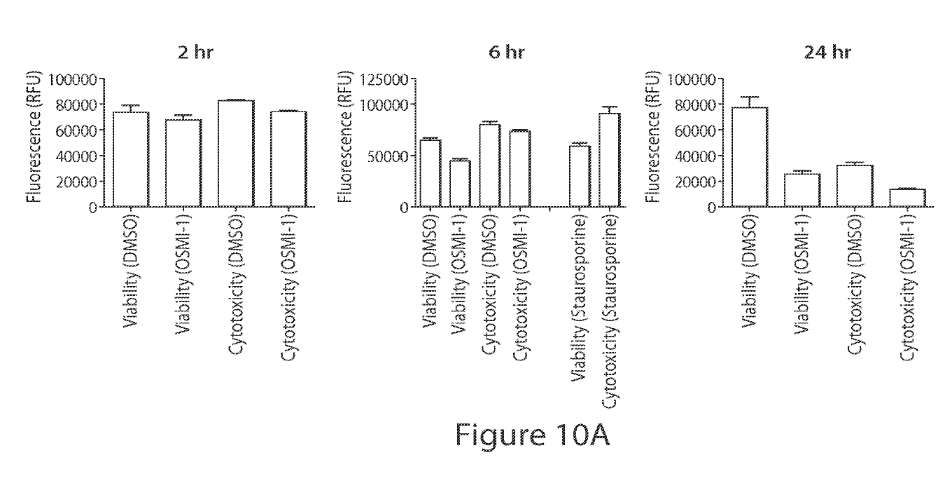

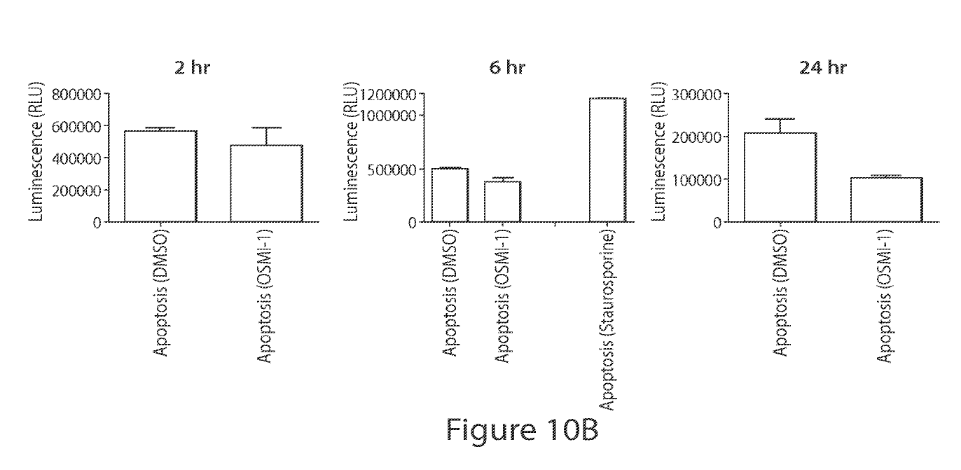

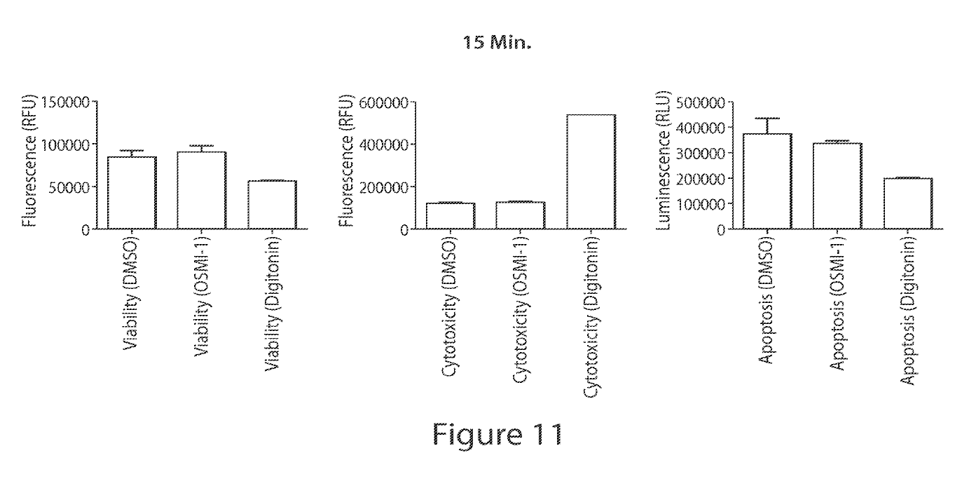

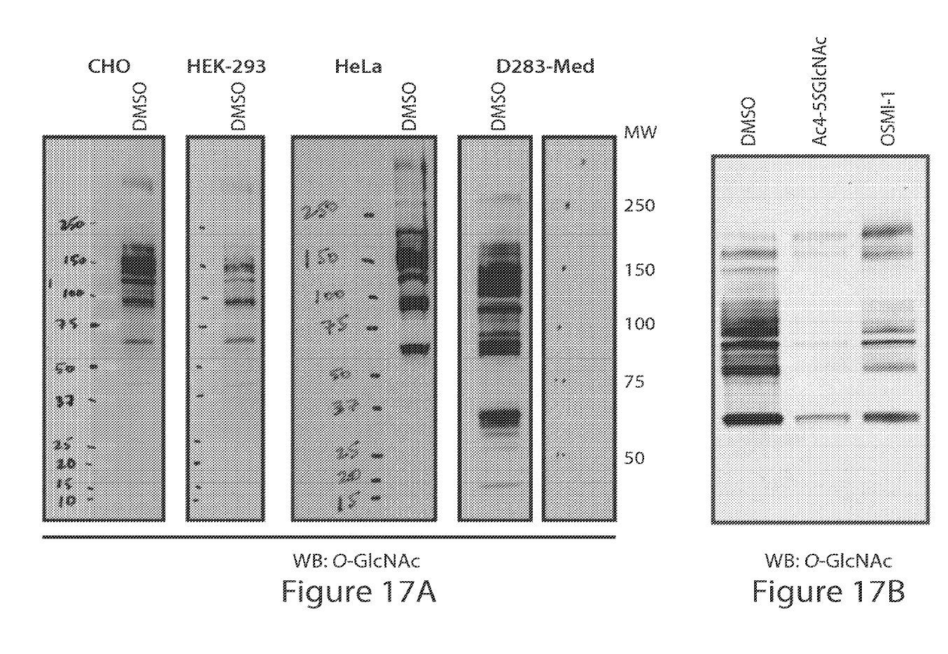

In an 8-hour time course study of CHO cells treated at 50 μM with either OSMI-1 or Ac4-5SGlcNAc, OSMI-1 showed a more rapid onset of O-GlcNAc reduction. A substantial reduction of global O-GlcNAcylation was observed for OSMI-1 within two hours, whereas Ac4-5SGlcNAc showed an effect only at four hours (FIG. 8). Several additional mammalian cell lines were examined and found that OSMI-1 treatment reduced global O-GlcNAcylation in all of them (FIGS. 9A and 17). Further, the effects of OSMI-1 were evaluated on specific cellular markers of OGT inhibition. Nup62 bears at least ten O-GlcNAc moieties that contribute over 2.5 kDa to the protein mass, and it was found that treating cells with OSMI-1 caused Nup62 to shift to a lower molecular weight, consistent with loss of the O-GlcNAc residues (FIG. 15B)³⁶. It is also known that levels of OGA, the glycosidase that removes O-GlcNAc residues from proteins, decreases when cellular O-GlcNAcylation is blocked^{25,37}. Since OSMI-1, like Ac4-5SGlcNAc, reduced cellular OGA without affecting cellular OGT levels (FIG. 15B). Hence, OSMI-1 functions to inhibit OGT activity in cells.

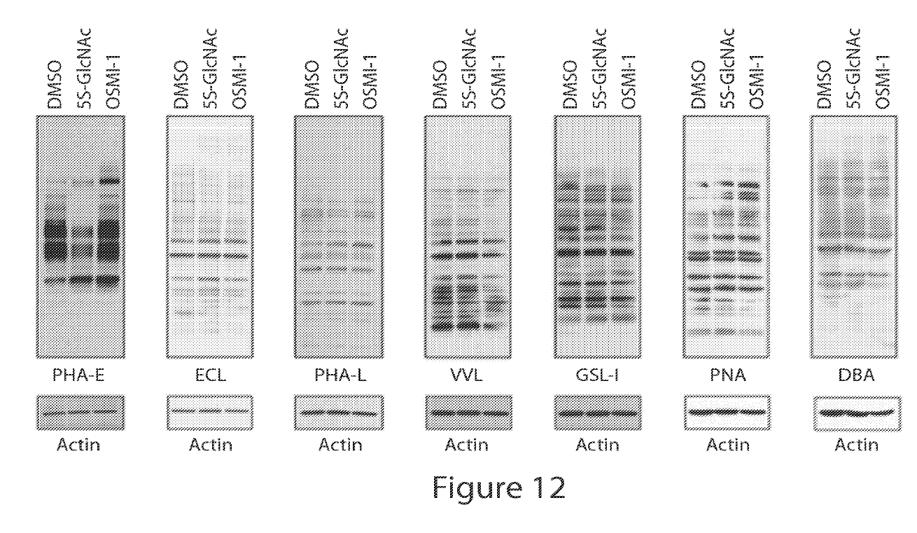

The selectivity of glycosyltransferase inhibitors wer evaluated by use of lectins to probe cell surface glycans following treatment of cells with a compound. Commercially available biotinylated lectins that recognize different features of N- and O-glycans are available for this purpose, and while their binding epitopes are not fully understood, they are useful for assessing whether a given treatment substantially alters glycan composition (FIG. 15C). Nine different biotinylated lectins (ConA, LCA, Jacalin, Pha-E, ECL, Pha-L, GSL-I, PNA or DBA) were used to probe the glycan composition of CHO cells treated with 50 μM OSMI-1 or Ac4-5SGlcNAc for 24 hours. For both compounds, minimal changes were observed in bands detected by ConA, PHA-L, ECL, GSL-I, PNA, or DBA, indicating that neither OSMI-1 nor Ac4-5SGlcNAc treatment grossly perturbed the carbohydrate structures recognized by these lectins (FIG. 15D; FIG. 12)²⁵. For Jacalin, PHA-E and LCA, however, dramatic changes were observed in the glycans from cells treated with Ac4-5SGlcNAc, but not from cells treated with OSMI-1 (FIG. 15D). Jacalin detects the GalNAc-peptide portion of mucin-type O-glycans, and Ac4-5SGlcNAc treatment resulted in decreased masses for several prominent bands (FIG. 15D)³⁸, suggesting that UDP-5SGlcNAc may block some glycosyltransferase(s) involved in mucin-type O-glycan synthesis. Ac4-5SGlcNAc treatment also resulted in greatly diminished signal intensity for the lectins PHA-E (FIG. 12) and LCA (FIG. 15D), suggesting that the inhibitor broadly affects several other types of cell surface glycans³⁹. Given the close resemblance of UDP-5SGlcNAc to substrates used by cellular glycosyltransferases, off-target effects for this inhibitor are perhaps not unexpected, particularly given its high intracellular concentratione.

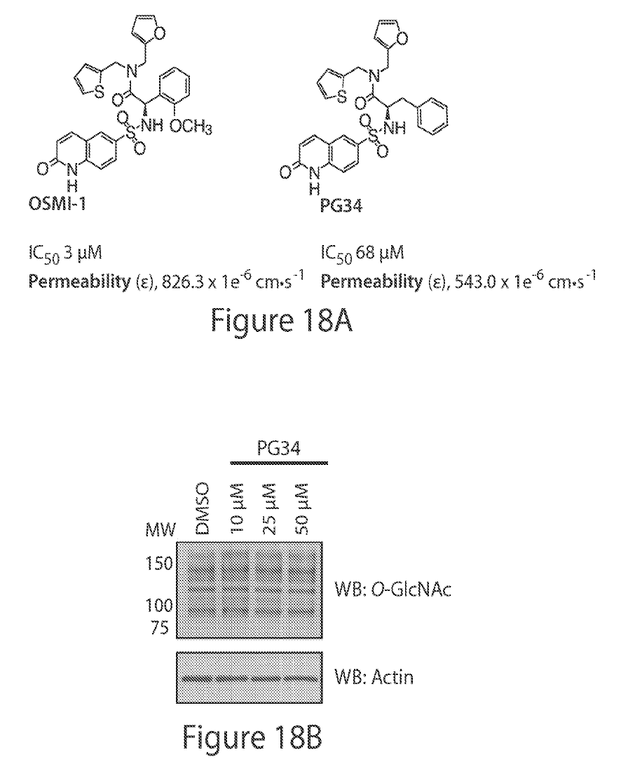

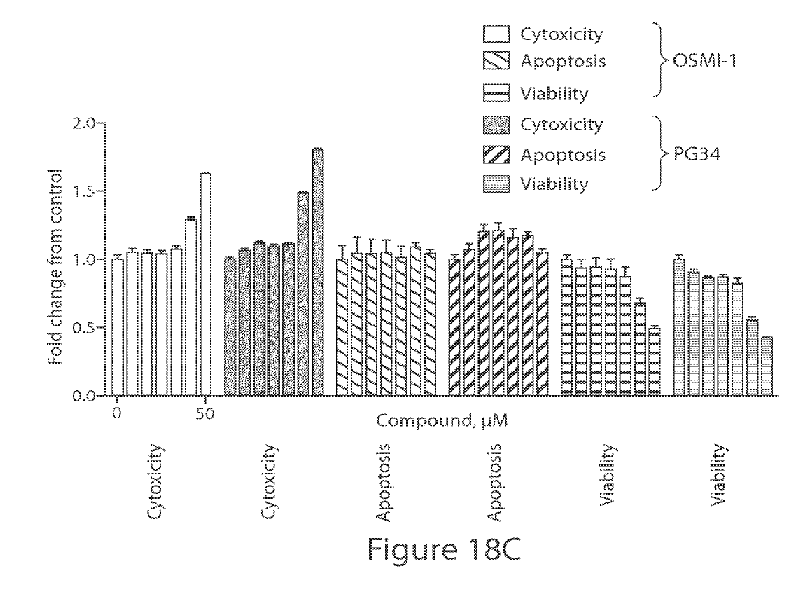

OGT is essential for development and remains essential in many cell types in both adult organisms and in in tissue culture⁴¹, but OGT inhibition by Ac4-5SGlcNAc was reported to have no effect on cell viability^{25, 42, 43}. The effects of 50 μM OSMI-1 on CHO cells were evaluated and it was found that viability decreased by about 50% after 24 hours (FIG. 18). In order to evaluate whether this effect resulted from inhibition of OGT or some other target, a structurally related compound was prepared. Compound PG34 (FIG. 18A) bears a phenylalanine in place of the 2-methoxyphenylglycine. PG34 demonstrated poor in vitro inhibitory activity against OGT and did not reduce global O-GlcNAcylation in cells (FIG. 18). However, it affected cell viability similarly to OSMI-1. Hence, it is possible that OSMI-1 shares a target other than OGT with PG34.

In summary, a cell-permeable small molecule OGT inhibitor was identified through a combination of high-throughput screening and follow-up chemistry. Through the use of a biased library and follow-up medicinal chemistry the potency of the initial screening hits (nearly 100×) was improved, and a compound was identified to inhibit OGT in cells⁴⁴. This compound was found to have on-target activity in cells based on its ability to reduce global O-GlcNAcylation, inhibit O-GlcNAcylation of cellular Nup62, and reduce OGA levels. Validation of other small molecule OGT inhibitors includes evaluation of Nup62 glycosylation, which is a convenient biomarker because the protein is ubiquitously expressed and so highly glycosylated that inhibition of O-GlcNAcylation results in a detectable mass shift. Although OSMI-1 is not as effective at reducing global O-GlcNAcylation at 24 hours as the same concentration of Ac4-5SGlcNAc, it has a more rapid onset of action owing to its ability to enter cells in an active state, and it does not appear to drastically alter other cellular glycans. OSMI-1 may thus be useful in conjunction with other inhibitors and methods for OGT inhibition/depletion to probe OGT inhibition phenotypes in cells.

### Example 3

Exemplary synthesis of compound OSMI-1.

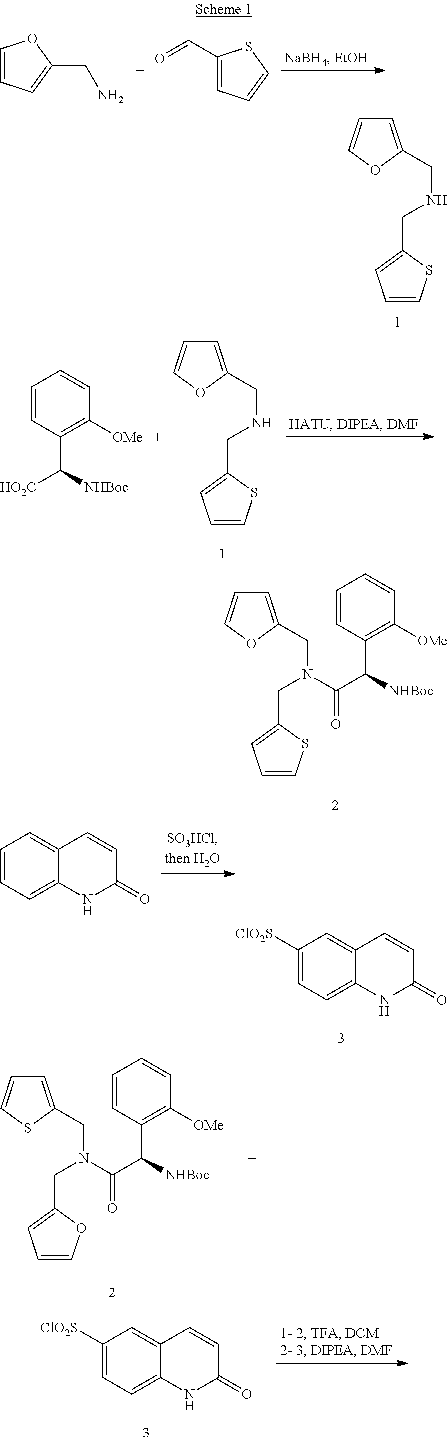

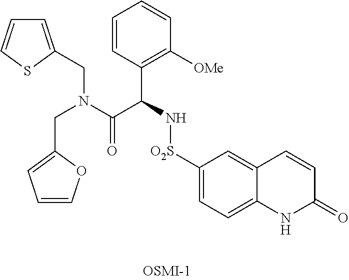 [SMILES: COc1ccccc1[C@@H](NS(=O)(=O)c1ccc2[nH]c(=O)ccc2c1)C(=O)N(Cc1ccco1)Cc1cccs1]

1-(furan-2-yl)-N-(thiophen-2-ylmethyl)methanamine (1)⁴⁴ (Deng, J.; Mo, L-P.; Zhao, F-Y.; Hou, L-L.; Yanga, L.; Zhang, Z-H. Green Chem. 2011, 9, 2576)

A mixture containing furan-2-ylmethanamine (1.00 mL, 11.32 mmol) and thiophene-2-carbaldehyde (1.06 mL, 11.32 mmol) in EtOH (22.6 mL) was heated in themicrowave reactor at 120° C. for 0.5 h.

The reaction solution was transferred to a round-bottomed flask, and was then treated with sodium borohydride (0.856 g, 22.63 mmol) at 90° C. for 3 h, then at 23° C. for 16 h. The reaction mixture was concentrated under reduced pressure, and the residue was partitioned between 50 mL of dichloromethane (DCM) and 50 mL of water. The product was extracted with two 25-mL portions of DCM and the combined organic layer was washed with 50 mL of brine, and subsequently dried over anhydrous sodium sulfate (Na₂SO₄). The dried organic layer was concentrated under reduced pressure and purified by silica gel column chromatography(100 g); gradient elution from 90:10 to 50:50 Hex:EtOAc afforded 1-(furan-2-yl)-N-(thiophen-2-ylmethyl)methanamine (1) as a clear light pale yellow oil; yield: 1.87 g (86%). LC-MS: t=1.77 min. ¹H NMR (400 MHz, CDCl₃) δ 7.38 (dd, J=1.9, 0.8 Hz, 1H), 7.22 (dd, J=4.8, 1.4 Hz, 1H), 6.98-6.92 (m, 2H), 6.32 (dd, J=3.2, 1.9 Hz, 1H), 6.22-6.18 (m, 1H), 3.99 (d, J=0.7 Hz, 2H), 3.82 (s, 2H), 1.98 (s, 2H). ¹³C NMR (101 MHz, CDCl₃) δ 153.5, 143.5, 142.1, 126.8, 125.4, 124.7, 110.3, 107.5, 47.2, 45.0.

HRMS (ESI) m/z 194.0635 (M+H)⁺ (C₁₀H₁₂NOS requires 194.0634).

### (R)-tert-butyl (2-((furan-2-ylmethyl)(thiophen-2-ylmethypamino)-1-(2-methoxyphenyl)-2-oxoethyl)carbamate (2)

A solution containing (R)-2-((tert-butoxycarbonyl)amino)-2-(2-methoxyphenyl)acetic acid (0.200 g, 0.711 mmol) and 1-(furan-2-yl)-N-(thiophen-2-ylmethyl)methanamine (1) (0.137 g, 0.711 mmol) in DMF (3.55 mL) was treated with HATU (0.297 g, 0.782 mmol) and N,N-diisopropylethylamine (0.137 ml, 0.782 mmol) at 23° C. for 5 h. The reaction mixture was partitioned between 40 mL of EtOAc and 40 mL of water. The product was extracted with three 20 mL portions of EtOAc. The combined organic layer was washed with two 20-mL of water, washed with with 20 mL of brine, dried over anhydrous sodium sulfate (Na₂SO₄), and concentrated under reduced pressure. The obtained oil was applied to a silica gel column (50-g); eluting from 80:20 to 40:60 Hex-EtOAc afforded (R)-tert-butyl (2-((furan-2-ylmethyl)(thiophen-2-ylmethyl)amino)-1-(2-methoxyphenyl)-2-oxoethyl)carbamate (2) as a clear colorless syrup and as a 60:40 mixture of rotamers; yield: 0.301 g (93%). LC-MS: t=6.17 min. ¹H NMR (400 MHz, CDCl₃) δ 7.34 (m, 0.6×2H), 7.31 (m, 0.4×2H), 7.30-7.23 (m, 1H), 7.18 (dd, J=5.0, 1.3 Hz, 0.6×1H), 7.15 (d, J=5.1 Hz, 0.4×1H), 6.97 (dd, J=5.1, 3.5 Hz, 0.4×1H), 6.93 (m, 0.6×1H), 6.89-6.81 (m, 2H+0.6×1H), 6.70 (m, 0.4×1H), 6.28 (dd, J=3.2, 1.9 Hz, 0.4×1H), 6.26 (dd, J=3.2, 1.9 Hz, 0.6×1H), 6.15 (d, J=3.2 Hz, 0.4×1H), 6.10 (d, J=7.5 Hz, 1H), 5.97 (d, J=3.2 Hz, 0.6×1H), 5.87 (dd, J=17.7, 8.4 Hz, 1H), 4.91 (d, J=15.2 Hz, 0.6×1H), 4.64 (s, 0.4×2H), 4.50 (dd, J=10.8, 5.6 Hz, 2H), 4.26 (d, J=16.5 Hz, 0.6×1H), 3.78 (s, 0.6×3H), 3.76 (s, 0.4×3H), 1.42 (s, 9H). ¹³C NMR (100 MHz, CDCl₃) δ 170.8, 170.8, 156.3, 155.0, 150.5, 149.8, 142.5, 142.3, 139.3, 138.7, 129.7, 129.6, 128.59, 128.53, 127.0, 126.9, 126.7, 126.5, 126.4, 126.2, 125.8, 125.6, 125.5, 125.5, 121.4, 121.3, 111.5, 111.3, 110.6, 110.4, 108.8, 108.5, 79.6, 55.9, 55.8, 49.6, 49.4, 44.7, 43.4, 42.5, 40.7, 28.5, 28.5, 28.5, 28.5, 28.5, 28.5. HRMS (ESI) m/z 457.1798 (M+H)⁺ (C₂₄H₂₉N₂O₅S requires 457.1792).

[α]_{D}²⁰=−53 (c 1.0, CHCl₃).

### 2-oxo-1,2-dihydroquinoline-6-sulfonyl chloride (3)⁴⁶ (WO 2010141074)

A mixture containing 1,2-dihydroquinolin-2-one (1.30 g, 8.96 mmol) in chlorosulfonic acid (3.90 ml, 58.7 mmol) was stirred at 90° C. for 3 h. The reaction mixture was allowed to cool down to room temperature, and was then poured carefully into 50 mL of crushed ice, leading to the formation of a precipitate. The solid was collected by filration, washed with small portions of cold water, and was then dried to afford 2-oxo-1,2-dihydroquinoline-6-sulfonyl chloride (3) as a light brown solid; yield: 1.52 g (70%). ¹H NMR (400 MHz, DMSO-d₆) δ 11.81 (s, 1H), 7.96 (d, J=9.4 Hz, 1H), 7.89 (d, J=1.9 Hz, 1H), 7.70 (dd, J=8.5, 1.9 Hz, 1H), 7.23 (d, J=8.5 Hz, 1H), 6.49 (d, J=9.5 Hz, 1H). ¹³C NMR (100 MHz, DMSO-d₆) δ 162.4, 142.5, 141.1, 139.2, 128.5, 125.3, 122.4, 118.5, 115.0.

### (R)—N-(furan-2-ylmethyl)-2-(2-methoxyphenyl)-2-(2-oxo-1,2-dihydroquinoline-6-sulfonamido)-N-(thiophen-2-ylmethyl)acetamide (OSMI-1, NCGC00344466)

A solution containing (R)-tert-butyl (2-((furan-2-ylmethyl)(thiophen-2-ylmethyl)amino)-1-(2-methoxyphenyl)-2-oxoethyl)carbamate (2) (0.788 g, 1.73 mmol) in DCM (volume: 19.2 mL) was treated with trifluoroacetic acid (1.33 mL, 17.3 mmol) at 23° C. for 1.5 h. The reaction mixture was concentrated under reduced pressure, then the residue was taken up in toluene, and the resulting mixture was concentrated again under reduced pressure. This process was repeated twice.

The obtained crude amine was dissolved into DMF (volume: 9.60 mL), and was then with treated with N,N-diisopropylethylamine (0.904 mL, 5.18 mmol) and with 2-oxo-1,2-dihydroquinoline-6-sulfonyl chloride (3) (0.631 g, 2.59 mmol) at 23° C. for 14 h. The reaction mixture was partitioned between 40 mL of water and 25 mL of EtOAc. The layers were separated, and the product was extracted with three 25-mL portions of EtOAc. The combined organic layers were washed with 25 mL of brine, dried over anhydrous sodium sulfate (Na₂SO₄), and then concentrated under reduced pressure. The resulting brown oil was applied to a silica gel column (80 g); gradient elution from 99:1 to 95:5 DCM-MeOH (with the MeOH containing 10% NH₄OH as modifier), followed by HPLC purification, afforded (R)—N-(furan-2-ylmethyl)-2-(2-methoxyphenyl)-2-(2-oxo-1,2-dihydroquinoline-6-sulfonamido)-N-(thiophen-2-ylmethyl)acetamide (OSMI-1) as a yellow solid and as a 60:40 mixture of rotamers; yield: 0.549 g (56%). LC-MS: t=5.10 min. ¹H NMR (400 MHz, DMSO-d₆) δ 11.95 (s, 1H), 8.43 (d, J=9.5 Hz, 0.4×1H), 8.41 (d, J=9.6 Hz, 0.6×1H), 7.88-7.84 (m, 2H), 7.65 (dd, J=1.8, 0.6 Hz, 0.6×1H), 7.61 (dd, J=8.7, 2.1 Hz, 0.6×1H), 7.60 (dd, J=8.7, 2.1 Hz, 0.4×1H), 7.49 (dd, J=1.8, 0.8 Hz, 0.4×1H), 7.45 (dd, J=5.1, 1.3 Hz, 0.4×1H), 7.32 (dd, J=5.0, 1.3 Hz, 0.6×1H), 7.22 (dd, J=7.6, 1.7 Hz, 0.6×1H), 7.20-7.15 (m, 1H+0.4×1H), 7.14-7.07 (m, 1H), 6.96 (dd, J=5.1, 3.4 Hz, 0.4×1H), 6.84 (m, 1H), 6.81 (dd, J=3.5, 1.3 Hz, 0.6×1H), 6.80-6.68 (m, 2H), 6.56 (d, J=9.6 Hz, 1H), 6.44 (dd, J=3.2, 1.9 Hz, 0.6×1H), 6.30 (dd, J=3.2, 1.9 Hz, 0.4×1H), 6.19 (d, J=3.2 Hz, 0.6×1H), 6.01 (d, J=3.2 Hz, 0.4×1H), 5.83 (d, J=9.6 Hz, 0.6×1H), 5.78 (d, J=9.5 Hz, 0.4×1H), 4.61 (d, J=15.1 Hz, 0.6×1H), 4.48 (s, 0.4×2H), 4.43-4.24 (m, 2H), 4.16 (d, J=16.7 Hz, 0.6×1H), 3.57 (s, 0.6×3H), 3.53 (s, 0.4×3H). ¹³C NMR (100 MHz, CDCl₃) δ 168.65, 168.62, 161.93, 161.93, 155.07, 155.05, 150.03, 149.8, 143.13, 143.11, 142.48, 142.47, 140.93, 140.91, 140.08, 140.04, 138.97, 138.85, 133.90, 133.87, 129.5, 129.5, 128.4, 128.3, 128.13, 128.06, 127.0, 126.9, 126.9, 126.5, 126.3, 126.1, 123.9, 123.8, 122.9, 122.9, 120.4, 120.4, 117.9, 117.9, 115.2, 115.2, 110.8, 110.8, 110.6, 110.6, 108.5, 108.2, 55.49, 55.46, 50.30, 50.11, 44.1, 43.5, 41.9, 41.1. HRMS (ESI) m/z 564.1253 (M +H)⁺ (C₂₈H₂₆N₃O₆S₂ requires 564.1258). [α]_{D}²⁰=−50 (c 1.0, CHCl₃).

### Example 4

Synthesis of PG34

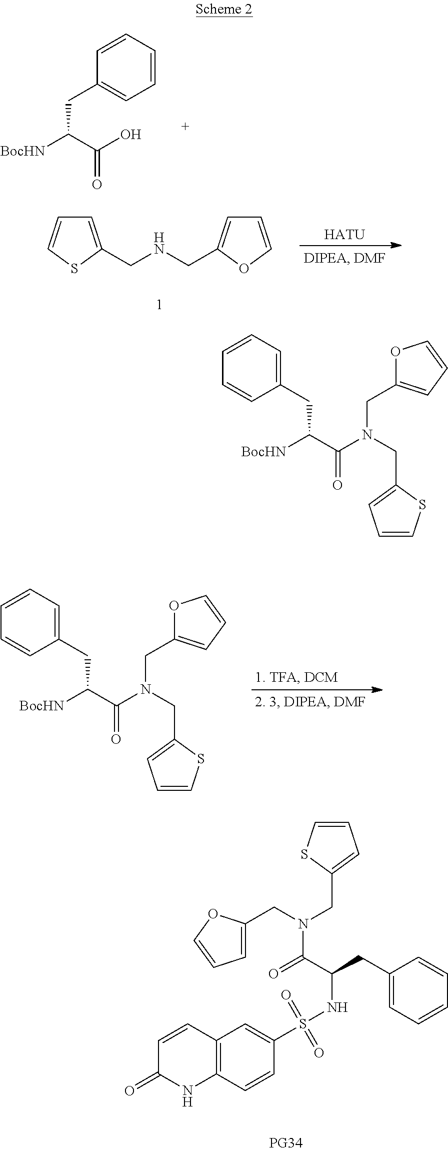

### (R)—N-(furan-2-ylmethyl)-2-(2-oxo-1,2-dihydroquinoline-6-sulfonamido)-3-phenyl-N-(thiophen-2-ylmethyl)propanamide (PG34, NCGC00345432)

A solution containing (R)-2-((tert-butoxycarbonyl)amino)-3-phenylpropanoic acid (200 mg, 0.754 mmol) and 1-(furan-2-yl)-N-(thiophen-2-ylmethyl)methanamine (1) (146 mg, 0.754 mmol) in 1.50 mL of DMF was treated sequentially with DIPEA (0.130 mL, 0.754 mmol) and HATU (287 mg, 0.754 mmol) at 23° C. for 16 h. The reaction mixture was concentrated, and the residue was applied to a silica gel column (50 g); gradient elution from 90:10 to 40:60 Hex:EtOAc afforded (R)-tert-butyl (1-((furan-2-ylmethyl)(thiophen-2-ylmethyl)amino)-1-oxo-3-phenylpropan-2-yl)carbamate as a white solid in 84% yield (280 mg).

A solution containing (R)-tert-butyl (1-((furan-2-ylmethyl)(thiophen-2-ylmethyl)amino)-1-oxo-3-phenylpropan-2-yl)carbamate (260 mg, 0.590 mmol) in 6.00 mL of DCM was treated with trifluoroacetic acid (0.455 mL, 5.90 mmol) at 23° C. for 1 h. The reaction mixture was concentrated under reduced pressure, and the residue was taken up in hexanes. The resulting suspension was then concentrated under reduced pressure to afford the crude amine. Without further purification, the amine was dissolved into 3.0 mL of DMF and the resulting solution was treated with DIPEA (150 μL, 0.859 mmol) and 2-oxo-1,2-dihydroquinoline-6-sulfonyl chloride (3) (152 mg, 0.624 mmol) at 23° C. for 16 h. The reaction mixture was concentrated, and the obtained residue was purified by HPLC to afford (R)—N-(furan-2-ylmethyl)-2-(2-oxo-1,2-dihydroquinoline-6-sulfonamido)-3-phenyl-N-(thiophen-2-ylmethyl)propanamide (PG34) as a white solid; yield: 122 mg (38%). LC-MS: t=5.48 min. ¹H NMR (400 MHz, DMSO-d₆) δ 12.00 (s, 1H), 8.39 (d, J=9.1 Hz, 0.4×1H), 8.36 (d, J=9.3 Hz, 0.6×1H), 7.89 (m, 2H), 7.62 (dd, J=2.0, 0.8 Hz, 0.6×1H), 7.59 (dd, J=6.1, 2.1 Hz, 0.6×1H), 7.56 (d, J=1.8, 0.9 Hz, 0.4×1H), 7.44 (dd, J=5.1, 1.2 Hz, 0.4×1H), 7.34 (dd, J=5.0, 1.4 Hz, 0.6×1H), 7.23 (d, J=8.7 Hz, 1H), 7.10-7.00 (m, 5H), 6.95 (dd, J=5.1, 3.5 Hz, 0.4×1H), 6.88 (dd, J=5.0, 3.4 Hz, 0.6×1H), 6.85 (dd, J=3.5, 1.3 Hz, 0.6×1H), 6.84-6.78 (m, 1H), 6.60 (dd, J=9.0, 1.9 Hz, 0.4×1H), 6.57 (dd, J=9.6, 1.9 Hz, 0.6×1H), 6.41 (dd, J=3.2, 1.9 Hz, 0.6×1H), 6.36 (dd, J=3.2, 1.8 Hz, 0.4×1H), 6.23 (d, J=3.2 Hz, 0.6×1H), 6.18 (d, J=3.2 Hz, 0.4×1H), 4.79 (d, J=17.2 Hz, 0.4×1H), 4.59-4.50 (m, 1H), 4.45 (m, 1H+0.6×1H), 4.37-4.24 (m, 2H), 2.82 (dd, J=13.7, 5.4 Hz, 0.6×1H), 2.72-2.54 (m, 0.6×1H+0.4×2H). ¹³C NMR (100 MHz, DMSO-d₆) δ 170.4, 170.4, 170.1, 170.1, 161.9, 150.1, 150.1, 149.7, 149.7, 143.1, 143.1, 142.6, 142.6, 141.1, 141.1, 140.1, 140.0, 139.9, 138.8, 136.5, 136.3, 134.0, 133.9, 129.3, 129.2, 127.93, 127.85, 127.8, 127.7, 127.1, 127.0, 126.7, 126.6, 126.3, 126.3, 126.2, 126.1, 125.91, 125.85, 123.1, 123,1, 118.20, 118.18, 115.7, 115.6, 110.6, 110.5, 108.7, 108.6, 54.03, 53.98, 43.6, 43.6, 42.8, 42.8, 41.8, 41.8, 37.93, 37.88. [α]_{D}²⁰=−196 (c 0.46, CHCl₃). HRMS (ESI) m/z 548.1298 [M+H]⁺ (C₂₈H₂₆N₃O₅S₂ requires 548.1308).

### Example 5

Purification of Nup62: Nup62 was PCR amplified from pET21A with primers 1 and 2 and ligated into a pET42a vector using the Spel and Notl sites and the final in-frame construct was verified by Sanger sequencing.

Primer JCJ0-12N1:
5′-GCTAGCACTAGTATGAGCGGGTTTAA-3′.
Primer JCJ0-12C:
5′-TAATATGCGGCCGCTTAGTCAAAGGT-3′.

GST-Nup62 was purified from E. coli BL21(DE3) as follows: LB media, supplemented with 50 μg/mL kanamycin, was warmed to 37° C. and inoculated with a 1:100 dilution of an overnight culture. Growth was monitored at OD₆₀₀ and once OD₆₀₀ reached approximately 0.4, overexpression was initiated by the addition of IPTG to 0.2 mM and allowed to proceed for 3 h at that temperature. After this time, cells were harvested by pelleting at 5,000×g for 20 min (at 4° C.). The cell pellets were then flash frozen in liquid nitrogen until needed. To obtain recombinant GST-Nup62, cell pellets were thawed on ice and resuspended in 10 mL of lysis buffer (50 mM Tris-HCl, pH 8.0, 10 mM EDTA, 0.5 M NaCl, 2 mM DTT and 1 mM PMSF; prepared fresh). Lysozyme was added to a final concentration of 0.1 mg/mL and the mixture was incubated on ice for 10 min. The mixture was diluted to 20 mL with additional lysis buffer and lysed by passing through a cell disruptor three times. Lysates were clarified by centrifugation at 10,000×g for 20 min (at 4° C.). The supernatant was transferred to a fresh tube and set aside. The pellet was washed by first resuspending in detergent solution A (1.1 M urea, 2% Triton X-100), followed by centrifugation at 10,000×g for 20 min as above. The pellet was washed twice to afford the purified inclusion body pellet, which was soluabilized by rocking at room temperature for several hours in 10 mL (for a cell pellet derived from ˜1.5 L of culture) denaturing buffer (6 M urea, 50 mM Tris-HCl, pH 8.0, 1 mM EDTA, 2 mM DTT; prepared fresh). After several hours, the supernatant was removed and the remaining pellet was solubilized in an additional 10 mL of denaturing buffer. The supernatant from both incubations was pooled and centrifuged at 16,100×g for 20 min to remove any solid debris. Solubilized GST-Nup62 was then dialyzed against 20 mM Tris-HCl pH 7.5 at 4° C. to remove urea and to remove urea and refold the protein, The dialyzed protein was pelleted at 16,100×g for 20 min to remove large aggregates, and the supernatant was further purified by GST-column chromatography following the manufacturer's specifications (Pierce). Affinity purified material was suitable for use without further

Purification of ncOGT: ncOGT was expressed and purified as described previously for hOGT⁴⁶, with the following exception: after Ni-NTA purification, cleavage of the N-terminal tags was not required. Instead, protein obtained from the elution fractions of the Ni-NTA column was directly purified by size-exclusion chromatography (Superdex 200) as described. ncOGT used in the experiments described below was quantified by nanodrop using an extinction coefficient, ε₂₈₀=118,955 M⁻¹ cm⁻¹.

### In Vitro Kinetics

Radiometric capture assay: Nucleoporin 62 (Nup62) was used as a model substrate. IC₅₀ values of the both inhibitors were determined at range of 0 to 100 μM (eight point, duplicate). OGT antagonists were tested in a reaction mixture containing 18 μM Nup62, 6 μM UDP-¹⁴C-GlcNAc, 100 nM ncOGT, and lx phosphate-buffered saline (1× PBS) (137 mM NaCl, 2.7 mM KCl, 10 mM Na₂HPO₄, and 1.8 mM KH₂PO₄, pH 7.4). The Nup62 glycosylation reactions were run for 15 min at 37° C. Reaction mixtures were subsequently quenched by spottinged on Whatman® Protran® nitrocellulose membrane, washed with 1× PBS three times for 15 minutes each. The membranes were air dried and then counted by liquid scintillation counting. The data were analyzed by Microsoft® Excel and Prism 6 (Graphpad®). Robust non-linear variable slope regressions were generated to determine the IC₅₀ and Hill slope values.

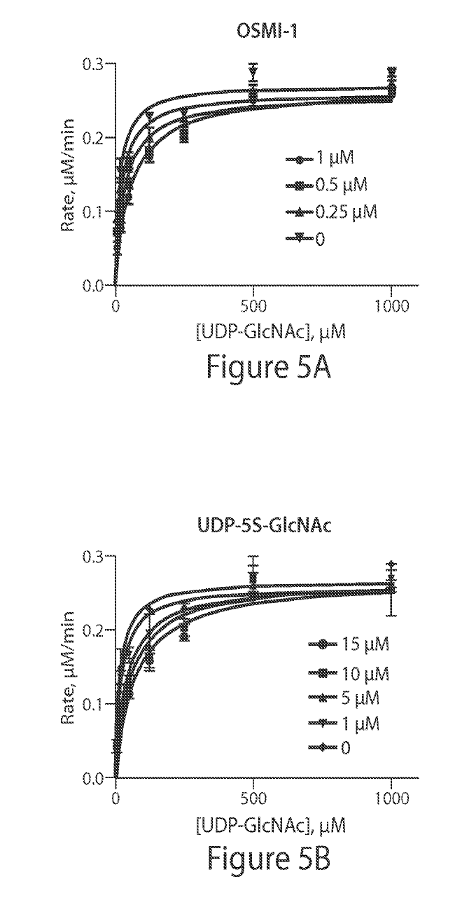

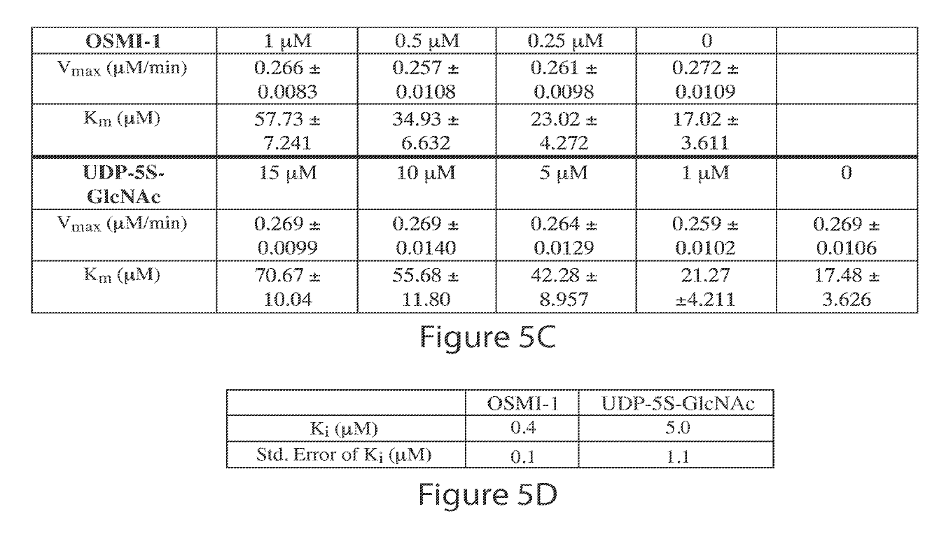

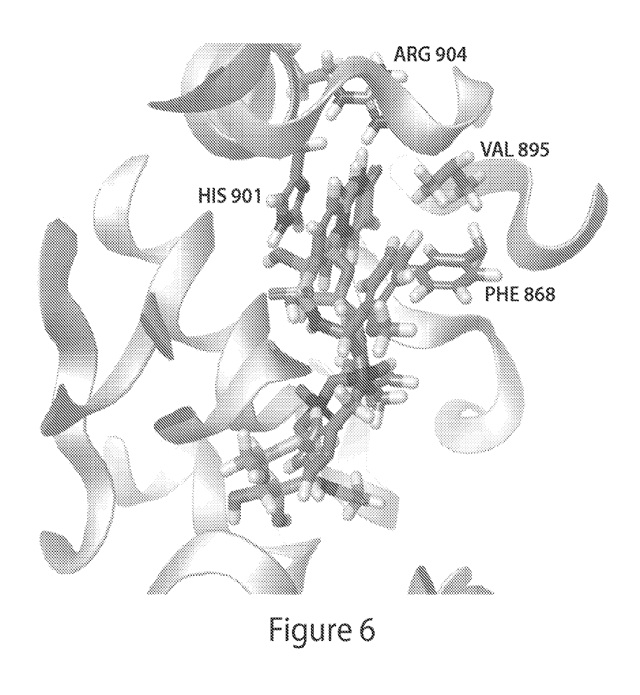

In order to evaluate the mode of inhibition of OSMI-1 and UDP-5S-GlcNAc, a series of experiments, as outlined above, were performed with Nup62 as glycosylation substrate. The inhibition test of OSMI-1 was performed by varying the UDP-¹⁴C-GlcNAc concentration from 0 to 0.25 mM while holding concentration of Nup62 constant at 10 μM with four different concentrations of OSMI-1 (0 to 2 μM) in 1× PBS with 100 nM ncOGT and with fixed and saturating concentrations of GST-Nup62 acceptor. The inhibition test of UDP-5S-GlcNAc was performed by varying the UDP-¹⁴C-GlcNAc concentration from 0 to 1 mM while holding concentration of Nup62 constant at 10 μM with five different concentrations of UDP-5S-GlcNAc (0, 1, 5, 10, and 15 μM) in 1× PBS with 100 nM ncOGT. Each reaction was spotted on Whatman® grade P81 ion exchange chromatography paper circles, washed with 1× PBS three times for 15 minutes each. The membranes were air dried and then counted by liquid scintillation counting. Each assay was duplicated in parallel. The data were analyzed by Microsoft® Excel and Prism 6 (Graphpad®). The Michaelis-Menten plots and Kᵢ of both inhibitors calculated under non-linear regression with competitive inhibition mode are shown in FIG. 5.

UDP-Glo assay: Materials for this assay were obtained from Promega (catalog #V6961), and the experiment was performed largely as outlined by the manufacturer. IC₅₀ values were determined in the range of 0 to 100 μM (eleven concentration points). Assays were performed either in white, 96-well or ½-volume 96-well, plates. This assay was run with 125 μM CKII3K peptide acceptor. Reaction volumes were between 14 (half volume plates) and 20 (normal volume plates) μL. Reactions contained the following components: 300 nM ncOGT, 125 μM CKII3K and 40 μM UDP-GlcNAc in 1× PBS pH 7.4 supplemented with 1 mM THP. Reactions were incubated for one hour at 25° C. and quenched by the addition of an equal volume of UDP-Glo nucleotide detection reagent, prepared and used according to manufacturer guidelines. The quenched reactions were then mixed briefly by shaking at 1800 rpm in a Thermomixer C, and incubated for one hour at room temperature prior to reading luminescence, as per the manufacturers guidelines. Data were analyzed by Microsoft Excel and Prism 6 (Graphpad). When evaluating the effect of sugar donor concentration, this parameter was varied, while all other conditions remained as described above. Each sample in this case was run in duplicate, unless otherwise specified. The 40 μM curve in FIG. 16C has each point in quadruplicate. Similarly, there is an n=9 for each point in the OSMI-1 curve in FIG. 16B.

While the IC₅₀ value for UDP-5SGlcNAc increases with increasing UDP-GlcNAc concentration, as expected for a competitive inhibitor (see FIG. 16 and Equation 1), the IC₅₀ value for OSMI-1 remains largely unchanged across a broad range of UDP-GlcNAc concentrations.

$${IC}_{50} = \frac{{K}_{i}}{{K}_{m}} \cdot [ S ] + {K}_{i} \qquad ( 1 )$$

Equation 1 is the Cheng-Prusoff equation. Rearranged such that IC₅₀ is the independent variable, [S] is the concentration of the competitive substrate (UDP-GlcNAc) and Kₘ is the Kₘ for UDP-GlcNAc. Given this relationship, IC₅₀ values should scale linearly with substrate concentration for competitive inhibitors.

We reason that OSMI-1 is not acting as a competitive inhibitor of OGT. From FIG. 16D, we see that the slope of the linear regression for the shift in OSMI-1 IC₅₀ is much too shallow to correspond to a competitive inhibitor.

In vivo inhibition of glycosylation: For all immunoblotting experiments, CHO-K1 (CHO) cells were grown in F12-K media supplemented with 10% FBS and antibiotics at 37° C. in a 5% CO₂ incubator. Cells were treated by adding indicated inhibitor in DMSO vehicle (0.5% final vehicle concentration) upon reaching 70% confluence. After indicated treatment length, cells were harvested by aspirating growth media and washing with ice cold 1× PBS. Cells were subsequently lysed in boiling 1% SDS/20 mM HEPES 7.9. After cooling to room temperature, 1 mM PMSF, 10 μM PUGNAC, 10 μM TMG and 1× Complete protease inhibitor cocktail were added to the solution. The mixture was sonicated and then subjected to a 5 minute incubation at 95° C. The samples were centrifuged for 15 minutes at 20,000×g and the supernatant was collected and the total protein was measured by BCA assay (BIO-RAD).

The sample was separated by SDS-PAGE (Bio-Rad 4-15%, Cat: 345-0028), transferred to a nitrocellulose membrane (iBlot, Invitrogen) and probed with the indicated antibody. Tyipcally, membranes were blocked with 4% BSA in 1× TBS+0.04% tween-20 for 1 hour before incubating with antibody (typically diluted 1:1000 in 4% BSA inlx TBS+0.04% tween-20) overnight at 4° C. Membranes were then washed three times with 1× TBS+0.04% tween-20 and incubated with secondary antibody (typically diluted 1:5000 in 4% BSA in 1× TBS+0.04% tween-20) for 1 hour at room temperature. Lastly, membranes were washed 3× with 1× TBS+0.04% tween-20 and imaged with ECL. D283-Med cells were treated as above but grown in F12-K media supplemented with 10% FBS and antibiotics. HEK, HeLa and LNCaP cells were treated as above but grown in RPMI media supplemented with 10% FBS and antibiotics.

It should be noted that HEK-293 cells were grown to >80% confluency prior to drug treatment. This was critical for obtaining a sufficiently high cell density post-treatment for subsequent immunoblotting.

Lectin blots were performed with the CHO-K1 cell lysate as prepared above. Lectin blots were typically blocked with lx Carbo-Free blocking solution (Vector Laboratories) prior to overnight incubation with biotinylated lectin at 4° C. The biotin label on the lectins was visualized with incubation with Streptavidin-HRP (Pierce) followed by ECL detection and imaging.

Cell viability, cytotoxicity and apoptosis signaling assays were performed in a 96-well plate using ApoTox-Glo reagent (Promega) according to the manufacturer's instructions.

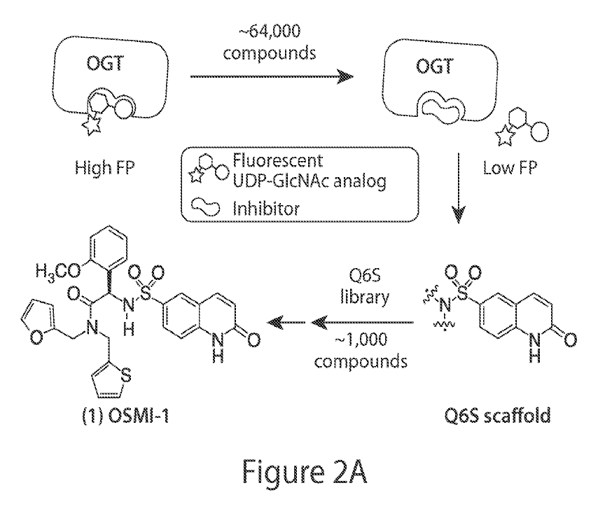

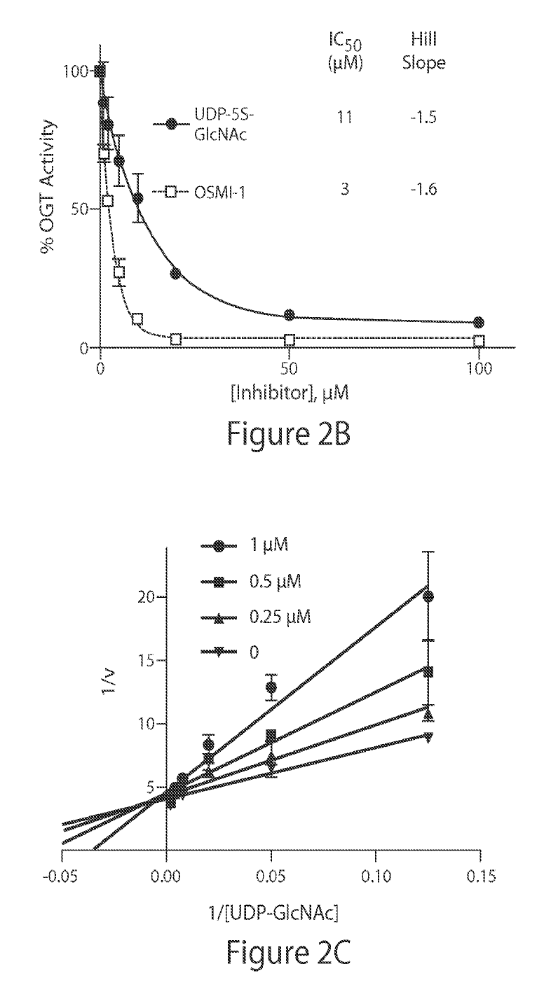

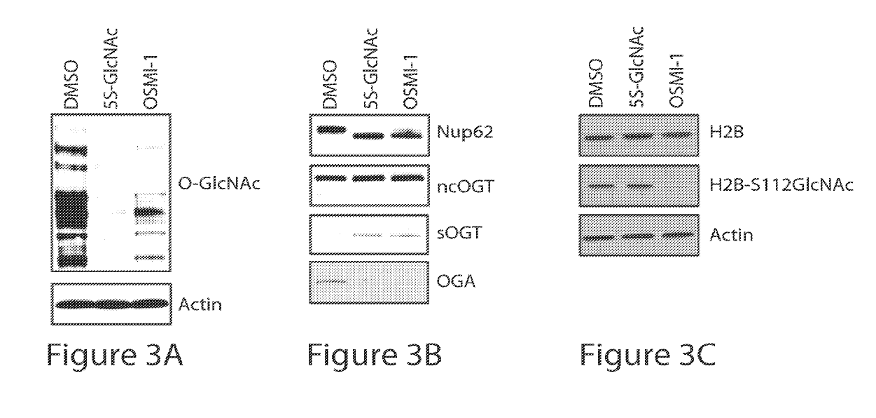

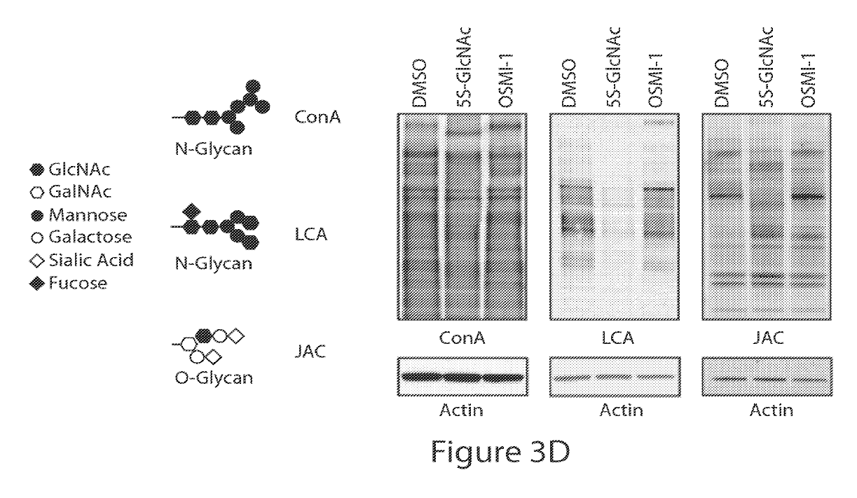

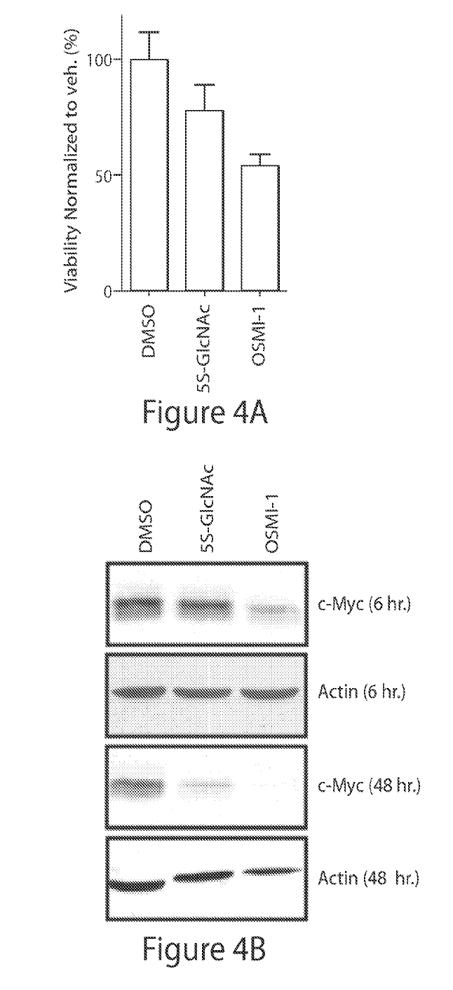

Induced-Fit Docking (IFD) Calculations: All calculations and following analyses were based on the crystal structure of the OGT (PDB: 4GYY) with the Schrodinger suite. Briefly, the structure was prepared with the Protein Preparation Wizard in Maestro version 9.6. OSMI-1 was first converted to smiles and then prepared using LigPrep 2.9 to avoid any conformational bias. Default settings were used unless stated otherwise. IFD calculations of the Q6S moiety in SI FIG. 2 was performed with the induced fit protocol in Maestro. In particular, Glide grid was centered on UDP-5S-GlcNAc and Glide redocking was performed using XP precision.

### REFERENCES

[1] Janetzko, J., and Walker, S. (2014) The making of a sweet modification: structure and function of O-GlcNAc transferase, The Journal of biological chemistry 289, 34424-34432.
[2] Hart, G. W., Housley, M. P., and Slawson, C. (2007) Cycling of O-linked beta-N-acetylglucosamine on nucleocytoplasmic proteins, Nature 446, 1017-1022.
[3] Hart, G. W., Slawson, C., Ramirez-Correa, G., and Lagerlof, O. (2011) Cross talk between O-GlcNAcylation and phosphorylation: roles in signaling, transcription, and chronic disease, Annu Rev Biochem 80, 825-858.
[4] Hanover, J. A., Krause, M. W., and Love, D. C. (2012) Bittersweet memories: linking metabolism to epigenetics through O-GlcNAcylation, Nat Rev Mol Cell Biol 13, 312-321.
[5] Love, D. C., and Hanover, J. A. (2005) The hexosamine signaling pathway: deciphering the “O-GlcNAc code”, Science's STKE : signal transduction knowledge environment 2005, re13.
[6] Wells, L., Kreppel, L. K., Comer, F. I., Wadzinski, B. E., and Hart, G. W. (2004) O-GlcNAc transferase is in a functional complex with protein phosphatase 1 catalytic subunits, The Journal of biological chemistry 279, 38466-38470.
[7] Tarrant, M. K., Rho, H. S., Xie, Z., Jiang, Y. L., Gross, C., Culhane, J. C., Yan, G., Qian, J., Ichikawa, Y., Matsuoka, T., Zachara, N., Etzkorn, F. A., Hart, G. W., Jeong, J. S., Blackshaw, S., Zhu, H., and Cole, P. A. (2012) Regulation of CK2 by phosphorylation and O-GlcNAcylation revealed by semisynthesis, Nature chemical biology 8, 262-269.
[8] Chu, C. S., Lo, P. W., Yeh, Y. H., Hsu, P. H., Peng, S. H., Teng, Y. C., Kang, M. L., Wong, C. H., and Juan, L. J. (2014) O-GlcNAcylation regulates EZH2 protein stability and function, Proceedings of the National Academy of Sciences of the United States of America 111, 1355-1360.
[9] Harwood, K. R., and Hanover, J. A. (2014) Nutrient-driven O-GlcNAc cycling—think globally but act locally, Journal of cell science 127, 1857-1867.
[10] Caldwell, S. A., Jackson, S. R., Shahriari, K. S., Lynch, T. P., Sethi, G., Walker, S., Vosseller, K., and Reginato, M. J. (2010) Nutrient sensor O-GlcNAc transferase regulates breast cancer tumorigenesis through targeting of the oncogenic transcription factor FoxM1, Oncogene 29, 2831-2842.
[11] Ma, Z., Vocadlo, D. J., and Vosseller, K. (2013) Hyper-O-GlcNAcylation is anti-apoptotic and maintains constitutive NF-kappaB activity in pancreatic cancer cells, The Journal of biological chemistry 288, 15121-15130.
[12] Bond, M. R., and Hanover, J. A. (2013) O-GlcNAc cycling: a link between metabolism and chronic disease, Annu Rev Nutr 33, 205-229.
[13] Slawson, C., and Hart, G. W. (2011) O-GlcNAc signalling: implications for cancer cell biology, Nature reviews. Cancer 11, 678-684.
[14] Yi, W., Clark, P. M., Mason, D. E., Keenan, M. C., Hill, C., Goddard, W. A., 3rd, Peters, E. C., Driggers, E. M., and Hsieh-Wilson, L. C. (2012) Phosphofructokinase 1 glycosylation regulates cell growth and metabolism, Science 337, 975-980.
[15] Itkonen, H. M., Minner, S., Guldvik, I. J., Sandmann, M. J., Tsourlakis, M. C., Berge, V., Svindland, A., Schlomm, T., and Mills, I. G. (2013) O-GlcNAc transferase integrates metabolic pathways to regulate the stability of c-MYC in human prostate cancer cells, Cancer Res 73, 5277-5287.
[16] Lynch, T. P., Ferrer, C. M., Jackson, S. R., Shahriari, K. S., Vosseller, K., and Reginato, M. J. (2012) Critical role of O-Linked beta-N-acetylglucosamine transferase in prostate cancer invasion, angiogenesis, and metastasis, The Journal of biological chemistry 287, 11070-11081.
[17] Prescher, J. A., and Bertozzi, C. R. (2006) Chemical technologies for probing glycans, Cell 126, 851-854.
[18] Kim, E. J. (2011) Chemical arsenal for the study of O-GlcNAc, Molecules 16, 1987-2022.
[19] Lee, T. N., Alborn, W. E., Knierman, M. D., and Konrad, R. J. (2006) Alloxan is an inhibitor of O-GlcNAc-selective N-acetyl-beta-D-glucosaminidase, Biochemical and biophysical research communications 350, 1038-1043.
[20] Lenzen, S., and Panten, U. (1988) Alloxan: history and mechanism of action, Diabetologia 31, 337-342.
[21] Goldberg, H., Whiteside, C., and Fantus, I. G. (2011) O-linked beta-N-acetylglucosamine supports p38 MAPK activation by high glucose in glomerular mesangial cells, American journal of physiology. Endocrinology and metabolism 301, E713-726.
[22] Wang, S., Shen, D. L., Lafont, D., Vercoutter-Edouart, A.-S., Mortuaire, M., Shi, Y., Maniti, O., Girard-Egrot, A., Lefebvre, T., Pinto, B. M., Vocadlo, D., and Vidal, S. (2014) Design of glycosyltransferase inhibitors targeting human O-GlcNAc transferase (OGT), Med Chem Comm 5, 1172-1178.
[23] Borodkin, V. S., Schimpl, M., Gundogdu, M., Rafie, K., Dorfmueller, H. C., Robinson, D. A., and van Aalten, D. M. (2014) Bisubstrate UDP-peptide conjugates as human O-GlcNAc transferase inhibitors, The Biochemical journal 457, 497-502.
[24] Dorfmueller, H. C., Borodkin, V. S., Blair, D. E., Pathak, S., Navratilova, I., and van Aalten, D. M. (2011) Substrate and product analogues as human O-GlcNAc transferase inhibitors, Amino acids 40, 781-792.
[25] Gloster, T. M., Zandberg, W. F., Heinonen, J. E., Shen, D. L., Deng, L., and Vocadlo, D. J. (2011) Hijacking a biosynthetic pathway yields a glycosyltransferase inhibitor within cells, Nature chemical biology 7, 174-181.
[26] Ferrer, C. M., Lynch, T. P., Sodi, V. L., Falcone, J. N., Schwab, L. P., Peacock, D. L., Vocadlo, D. J., Seagroves, T. N., and Reginato, M. J. (2014) O-GlcNAcylation regulates cancer metabolism and survival stress signaling via regulation of the HIF-1 pathway, Molecular cell 54, 820-831.
[27] Gross, B. J., Kraybill, B. C., and Walker, S. (2005) Discovery of O-GlcNAc transferase inhibitors, Journal of the American Chemical Society 127, 14588-14589.
[28] Gross, B. J., Swoboda, J. G., and Walker, S. (2008) A strategy to discover inhibitors of O-linked glycosylation, Journal of the American Chemical Society 130, 440-441.
[29] Helm, J. S., Hu, Y., Chen, L., Gross, B., and Walker, S. (2003) Identification of active-site inhibitors of MurG using a generalizable, high-throughput glycosyltransferase screen, Journal of the American Chemical Society 125, 11168-11169.
[30] Hu, Y., Helm, J. S., Chen, L., Ginsberg, C., Gross, B., Kraybill, B., Tiyanont, K., Fang, X., Wu, T., and Walker, S. (2004) Identification of selective inhibitors for the glycosyltransferase MurG via high-throughput screening, Chemistry & biology 11, 703-711.
[31] Gross, B. J. Discovery of O-GlcNAc Transferase Inhibitors. Ph.D. Dissertation, Harvard University, Cambridge, Mass,, 2007.
[32] Kim do, H., Seok, Y. M., Kim, I. K., Lee, I. K., Jeong, S. Y., and Jeoung, N. H. (2011) Glucosamine increases vascular contraction through activation of RhoA/Rho kinase pathway in isolated rat aorta, BMB reports 44, 415-420.
[33] Jiang, J., Lazarus, M. B., Pasquina, L., Sliz, P., and Walker, S. (2012) A neutral diphosphate mimic crosslinks the active site of human 0-GlcNAc transferase, Nature chemical biology 8, 72-77.
[34] Holt, G. D., Snow, C. M., Senior, A., Haltiwanger, R. S., Gerace, L., and Hart, G. W. (1987) Nuclear pore complex glycoproteins contain cytoplasmically disposed O-linked N-acetylglucosamine, J Cell Biol 104, 1157-1164.
[35] RL2 does not show any affinity to N-Linked Glycans. See: Reeves, R. A.; Lee, A.; Henry, R.; Zachara, N. E., Anal Biochem 2014, 457, 8-18.
[36] Ortiz-Meoz, R. F., Merbl, Y., Kirschner, M. W., and Walker, S. (2014) Microarray discovery of new OGT substrates: the medulloblastoma oncogene OTX2 is O-GlcNAcylated, Journal of the American Chemical Society 136, 4845-4848.
[37] Slawson, C., Zachara, N. E., Vosseller, K., Cheung, W. D., Lane, M. D., and Hart, G. W. (2005) Perturbations in O-linked beta-N-acetylglucosamine protein modification cause severe defects in mitotic progression and cytokinesis, The Journal of biological chemistry 280, 32944-32956.
[38] Bourne, Y., Astoul, C. H., Zamboni, V., Peumans, W. J., Menu-Bouaouiche, L., Van Damme, E. J., Barre, A., and Rouge, P. (2002) Structural basis for the unusual carbohydrate-binding specificity of jacalin towards galactose and mannose, The Biochemical journal 364, 173-180.
[39] Tateno, H., Nakamura-Tsuruta, S., and Hirabayashi, J. (2009) Comparative analysis of core-fucose-binding lectins from Lens culinaris and Pisum sativum using frontal affinity chromatography, Glycobiology 19, 527-536.
[40] Dube, D. H., Prescher, J. A., Quang, C. N., and Bertozzi, C. R. (2006) Probing mucin-type O-linked glycosylation in living animals, Proceedings of the National Academy of Sciences of the United States of America 103, 4819-4824.
[41] Watson, L. J., Long, B. W., DeMartino, A. M., Brittian, K. R., Readnower, R. D., Brainard, R. E., Cummins, T. D., Annamalai, L., Hill, B. G., and Jones, S. P. (2014) Cardiomyocyte Ogt is essential for postnatal viability, American journal of physiology. Heart and circulatory physiology 306, H142-153.
[42] Kazemi, Z., Chang, H., Haserodt, S., McKen, C., and Zachara, N. E. (2010) O-linked beta-N-acetylglucosamine (O-GlcNAc) regulates stress-induced heat shock protein expression in a GSK-3beta-dependent manner, The Journal of biological chemistry 285, 39096-39107.
[43] O'Donnell, N., Zachara, N. E., Hart, G. W., and Marth, J. D. (2004) Ogt-dependent X-chromosome-linked protein glycosylation is a requisite modification in somatic cell function and embryo viability, Mol Cell Biol 24, 1680-1690.
[44] Deng, J., Mo, L.-P., Zhao, F.-Y., Hou, L.-L., Yang, L., and Zhang, Z.-H. (2011) Sulfonic acid supported on hydroxyapatite-encapsulated4gammaFFe203 nanocrystallites as a magnetically separable catalyst for one-pot reductive amination of carbonyl compounds, Green Chemistry 13, 2576-2584.
[45] Kahne, S. W., Lazarus, M. B., and Gross, B. J. (2012) O-glcnac transferase inhibitors and uses thereof, Google Patents.
[46] Lazarus, M. B., Nam, Y., Jiang, J., Sliz, P., and Walker, S. (2011) Structure of human O-GlcNAc transferase and its complex with a peptide substrate, Nature 469, 564-567.
[47] Dauphinee, S. M., Ma, M., and Too, C. K. (2005) Role of O-linked beta-N-acetylglucosamine modification in the subcellular distribution of alpha4 phosphoprotein and Sp1 in rat lymphoma cells, J. Cell. Biochem. 96, 579-588.
[48] Konrad, R. J., Zhang, F., Hale, J. E., Knierman, M. D., Becker, G. W., and Kudlow, J. E. (2002) Alloxan is an inhibitor of the enzyme O-linked N-acetylglucosamine transferase, Biochem. Biophys. Res. Commun. 293, 207-212.
[49] Deplus, R., Delatte, B., Schwinn, M. K., Defrance, M., Mendez, J., Murphy, N., Dawson, M. A., Volkmar, M., Putmans, P., Calonne, E., Shih, A. H., Levine, R. L., Bernard, O., Mercher, T., Solary, E., Urh, M., Daniels, D. L., and Fuks, F. (2013) TET2 and TET3 regulate GlcNAcylation and H3K4 methylation through OGT and SET1/COMPASS, EMBO J. 32, 645-655.
[50] Guo, B., Liang, Q., Li, L., Hu, Z., Wu, F., Zhang, P., Ma, Y., Zhao, B., Kovacs, A. L., Zhang, Z., Feng, D., Chen, S., and Zhang, H. (2014) 0-GlcNAc-modification of SNAP-29 regulates autophagosome maturation, Nat Cell Biol 16, 1215-1226.
[51] Dehennaut, V., Lefebvre, T., Sellier, C., Leroy, Y., Gross, B., Walker, S., Cacan, R., Michalski, J. C., Vilain, J. P., and Bodart, J. F. (2007) O-linked N-acetylglucosaminyltransferase inhibition prevents G2/M transition in Xenopus laevis oocytes, J. Biol. Chem. 282, 12527-12536.
[52] Liu, J., Marchase, R. B., and Chatham, J. C. (2007) Glutamine-induced protection of isolated rat heart from ischemia/reperfusion injury is mediated via the hexosamine biosynthesis pathway and increased protein O-GlcNAc levels, J Mol Cell Cardiol 42, 177-185.
[53] Xu, C., Liu, G., Liu, X., and Wang, F. (2014) O-GlcNAcylation under hypoxic conditions and its effects on the blood-retinal barrier in diabetic retinopathy, Int J Mol Med 33, 624-632.
[54] Chen, C. L., Liang, C. M., Chen, Y. H., Tai, M. C., Lu, D. W., and Chen, J. T. (2012) Glucosamine modulates TNF-alpha-induced ICAM-1 expression and function through O-linked and N-linked glycosylation in human retinal pigment epithelial cells, Invest Ophthalmol Vis Sci 53, 2281-2291.
[55] Pantaleon, M., Tan, H. Y., Kafer, G. R., and Kaye, P. L. (2010) Toxic effects of hyperglycemia are mediated by the hexosamine signaling pathway and o-linked glycosylation in early mouse embryos, Biol Reprod 82, 751-758.
[56] Frank, L. A., Sutton-McDowall, M. L., Brown, H. M., Russell, D. L., Gilchrist, R. B., and Thompson, J. G. (2014) Hyperglycaemic conditions perturb mouse oocyte in vitro developmental competence via beta-O-linked glycosylation of heat shock protein 90, Hum Reprod 29, 1292-1303.
[57] Kang, E. S., Han, D., Park, J., Kwak, T. K., Oh, M. A., Lee, S. A., Choi, S., Park, Z. Y., Kim, Y., and Lee, J. W. (2008) O-GlcNAc modulation at Aktl Ser473 correlates with apoptosis of murine pancreatic beta cells, Exp Cell Res 314, 2238-2248.
[58] Olivier-Van Stichelen, S., Drougat, L., Dehennaut, V., El Yazidi-Belkoura, I., Guinez, C., Mir, A. M., Michalski, J. C., Vercoutter-Edouart, A. S., and Lefebvre, T. (2012) Serum-stimulated cell cycle entry promotes ncOGT synthesis required for cyclin D expression, Oncogenesis 1, e36.
[59] Lima, V. V., Giachini, F. R., Carneiro, F. S., Carvalho, M. H., Fortes, Z. B., Webb, R. C., and Tostes, R. C. (2011) O-GlcNAcylation contributes to the vascular effects of ET-1 via activation of the RhoA/Rho-kinase pathway, Cardiovasc Res 89, 614-622.
[60] Jeon, J. H., Suh, H. N., Kim, M. O., and Han, H. J. (2013) Glucosamine-induced reduction of integrin beta4 and plectin complex stimulates migration and proliferation in mouse embryonic stem cells, Stem Cells Dev 22, 2975-2989.
[61] Lima, V. V., Lobato, N. S., Filgueira, F. P., Webb, R. C., Tostes, R. C., and Giachini, F. R. (2014) Vascular O-GlcNAcylation augments reactivity to constrictor stimuli by prolonging phosphorylated levels of the myosin light chain, Braz J Med Biol Res 47, 826-833.
[62] Jeon, J. H., Suh, H. N., Kim, M. O., Ryu, J. M., and Han, H. J. (2014) Glucosamine-induced OGT activation mediates glucose production through cleaved Notchl and FoxO1, which coordinately contributed to the regulation of maintenance of self-renewal in mouse embryonic stem cells, Stem Cells Dev 23, 2067-2079.
[63] Suh, H. N., Lee, Y. J., Kim, M. O., Ryu, J. M., and Han, H. J. (2014) Glucosamine-induced Sp1 O-GlcNAcylation ameliorates hypoxia-induced SGLT dysfunction in primary cultured renal proximal tubule cells, J. Cell. Physiol. 229, 1557-1568.
[64] Shi, Y., Tomic, J., Wen, F., Shaha, S., Bahlo, A., Harrison, R., Dennis, J. W., Williams, R., Gross, B. J., Walker, S., Zuccolo, J., Deans, J. P., Hart, G. W., and Spaner, D. E. (2010) Aberrant O-GlcNAcylation characterizes chronic lymphocytic leukemia, Leukemia 24, 1588-1598.
[65] Tomic, J., McCaw, L., Li, Y., Hough, M. R., Ben-David, Y., Moffat, J., and Spaner, D. E. (2013) Resveratrol has anti-leukemic activity associated with decreased O-GlcNAcylated proteins, Exp Hematol 41, 675-686.
[66] Nagae, M., Soga, K., Morita-Matsumoto, K., Hanashima, S., Ikeda, A., Yamamoto, K., and Yamaguchi, Y. (2014) Phytohemagglutinin from Phaseolus vulgaris (PHA-E) displays a novel glycan recognition mode using a common legume lectin fold, Glycobiology 24, 368-378.
[67] Wu, A. M., Wu, J. H., Tsai, M. S., Yang, Z., Sharon, N., and Herp, A. (2007) Differential affinities of Erythrina cristagalli lectin (ECL) toward monosaccharides and polyvalent mammalian structural units, Glycoconj J 24, 591-604.
[68] Kaneda, Y., Whittier, R. F., Yamanaka, H., Carredano, E., Gotoh, M., Sota, H., Hasegawa, Y., and Shinohara, Y. (2002) The high specificities of Phaseolus vulgaris erythro- and leukoagglutinating lectins for bisecting GlcNAc or beta 1-6-linked branch structures, respectively, are attributable to loop B, J. Biol. Chem. 277, 16928-16935.
[69] Nash, R., Neves, L., Faast, R., Pierce, M., and Dalton, S. (2007) The lectin Dolichos biflorus agglutinin recognizes glycan epitopes on the surface of murine embryonic stem cells: a new tool for characterizing pluripotent cells and early differentiation, Stem Cells 25, 974-982.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A compound of Formula (I): 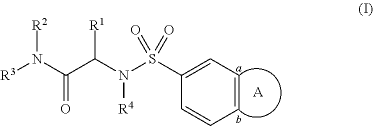
or a pharmaceutically acceptable salt thereof,
wherein
Ring A is of the formula 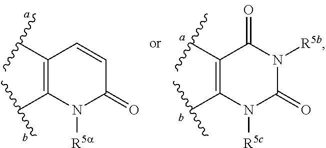
wherein a and b indicate the points of attachment to the phenyl ring;
R¹ is n-butyl, thiophene, —CH₂-Ph, cyclohexyl, or of the formula: 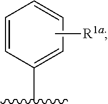
R^{1a} is hydrogen, halogen, —OR⁰, or optionally substituted C₁₋₄ alkyl;
R⁰ is hydrogen or C₁₋₄ alkyl;
each of R² and R³ is independently hydrogen, optionally substituted C₁₋₄ alkyl, optionally substituted thiophenyl-C₁₋₄ alkylene, optionally substituted phenyl-C₁₋₄ alkylene, or optionally substituted furanyl-C₁₋₄ alkylene;
R⁴ is hydrogen, optionally substituted C₁₋₆ alkyl, or a nitrogen protecting group;
each of R^{5a}, R^{5b}, and R^{5c} is independently hydrogen, optionally substituted C₁₋₆ alkyl, or a nitrogen protecting group;
R¹ and R⁴ may optionally be taken together with the intervening nitrogen to form optionally substituted heteroaryl or optionally substituted heterocycle; and
R² and R³ may optionally be taken together with the intervening nitrogen to form optionally substituted six-membered heterocycle.

2. The compound of claim 1 of Formula (II): 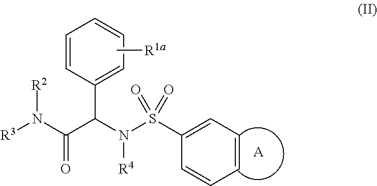
or a pharmaceutically acceptable salt thereof.

3. The compound of claim 2 of Formula (II-a): 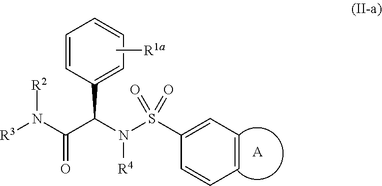
or a pharmaceutically acceptable salt thereof.

4. The compound of claim 2 of Formula (II-b): 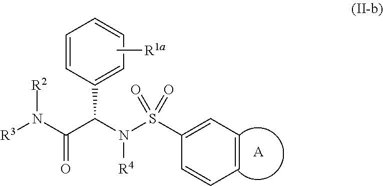
or a pharmaceutically acceptable salt thereof.

5. The compound of claim 1 of Formula (III): 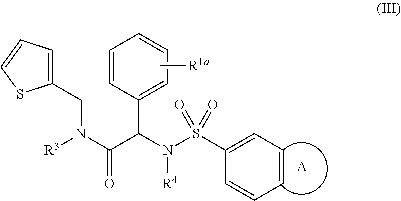
or a pharmaceutically acceptable salt thereof.

6. The compound of claim 5 of Formula (III-a): 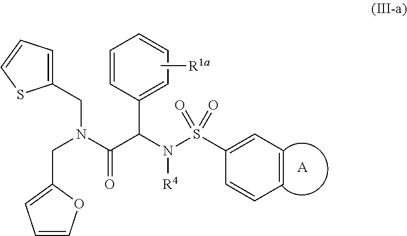
or a pharmaceutically acceptable salt thereof.

7. The compound of claim 6 of Formula (III-a1): 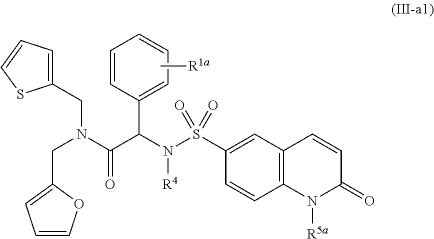
or a pharmaceutically acceptable salt thereof.

8. The compound of claim 7 of Formula (III-a1-i): 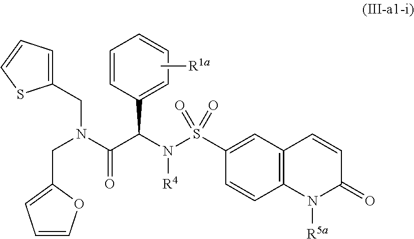
or a pharmaceutically acceptable salt thereof.

9. A compound of claim 7 of Formula (III-a1-ii): 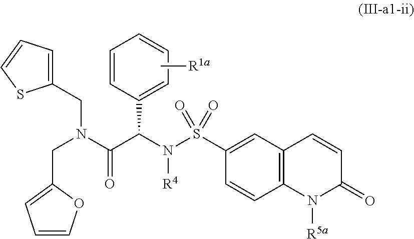
or a pharmaceutically acceptable salt thereof.

10. The compound of claim 6 of Formula (III-a2): 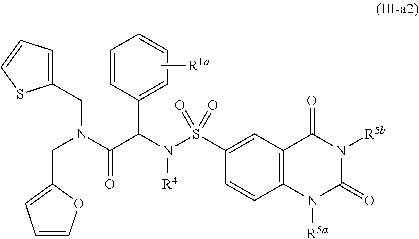
or a pharmaceutically acceptable salt thereof.

11. The compound of claim 10 of Formula (III-a2-i): 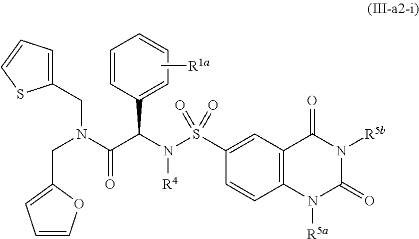
or a pharmaceutically acceptable salt thereof.

12. The compound of claim 10 of Formula (III-a2-ii): 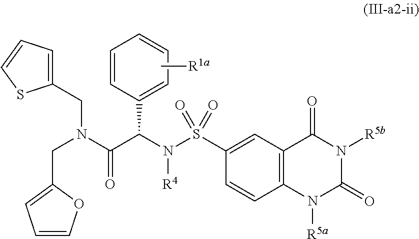
or a pharmaceutically acceptable salt thereof.

13. The compound of claim 1, wherein R¹ is n-butyl.

14. The compound of claim 1, wherein R¹ is —CH₂-Ph.

15. The compound of claim 1, wherein each of R² and R³ is independently optionally substituted thiophenyl-C₁₋₄ alkylene, optionally substituted phenyl-C₁₋₄ alkylene, or optionally substituted furanyl-C₁₋₄ alkylene.

16. The compound of claim 1, wherein each of R² and R³ is independently optionally substituted thiophenyl-C₁₋₄ alkylene or optionally substituted furanyl-C₁₋₄ alkylene.

17. The compound of claim 1, wherein each of R² and R³ is independently optionally substituted thiophenyl-CH₂—, optionally substituted phenyl-CH₂—, or optionally substituted furanyl-CH₂—.

18. is The compound of claim 1, wherein each of R² and R³ independently optionally substituted thiophenyl-CH₂— or optionally substituted furanyl-CH₂—.

19. The compound of claim 1, wherein R² is hydrogen; and R³ is C₁₋₄ alkyl.

20. The compound of claim 1, wherein R² is hydrogen; and R³ is methyl or ethyl.

21. The compound of claim 1, wherein R² is C₁₋₄ alkyl; and R³ is optionally substituted thiophenyl-C₁₋₄alkylene, optionally substituted phenyl-C₁₋₄ alkylene, or optionally substituted furanyl-C₁₋₄ alkylene.

22. R³ The compound of claim 1, wherein R² is C₁₋₄ alkyl; and is optionally substituted thiophenyl-C₁₋₄ alkylene or optionally substituted furanyl-C₁₋₄ alkylene.

23. The compound of claim 1, wherein R² is methyl; and R³ is optionally substituted thiophenyl-CH₂—, optionally substituted phenyl-CH₂—, or optionally substituted furanyl-CH₂—.

24. The compound of claim 1, wherein R² is methyl; and R³ is optionally substituted thiophenyl-CH₂— or optionally substituted furanyl-CH₂—.

25. The compound of claim 1, wherein R² and R³ are taken together with the intervening nitrogen to form optionally substituted six-membered heterocycle.

26. The compound of claim 1, wherein R² and R³ are taken together with the intervening nitrogen to form optionally substituted piperidinyl, piperazinyl, or morpholinyl ring.

27. The compound of claim 1, wherein R^{1a} is halogen.

28. The compound of claim 1, wherein R^{1a} is optionally substituted C₁₋₄ alkyl.

29. The compound of claim 1, wherein R^{1a} is substituted C₁₋₄ alkyl.

30. The compound of claim 1, wherein R^{1a} is —CF₃.

31. The compound of claim 1, wherein R^{1a} is unsubstituted C₁₋₄ alkyl.

32. The compound of claim 1, wherein R^{1a} is —CH₃.

33. The compound of claim 1, wherein R^{1a} is —OR⁰.

34. The compound of claim 1, wherein R^{1a} is —OH or —OCH₃.

35. The compound of claim 1, wherein R⁴ is hydrogen.

36. The compound of claim 1, wherein R⁴ is optionally substituted C₁₋₆ alkyl.

37. The compound of claim 1, wherein R¹ and R⁴ are taken together with the intervening nitrogen to form optionally substituted heteroaryl or optionally substituted heterocycle.

38. The compound of claim 37, wherein R¹ and R⁴ are taken together with the intervening nitrogen to form optionally substituted 5,6-membered heterocycle.

39. The compound of claim 37, wherein R¹ and R⁴ are taken together with the intervening nitrogen to form an optionally substituted isoindoline ring.

40. The compound of claim 37, wherein R¹ and R⁴ are taken together with the intervening nitrogen to form optionally substituted 6,6-membered heterocycle.

41. The compound of claim 37, wherein R¹ and R⁴ are taken together with the intervening nitrogen to form an optionally substituted dihydro-isoquinoline ring.

42. The compound of any preceding claims, wherein R^{5a} is hydrogen.

43. The compound of any preceding claims, wherein R^{5b} is hydrogen.

44. The compound of any preceding claims, wherein R^{5c} is hydrogen.

45. The compound of claim 1 of one of the following formulae:

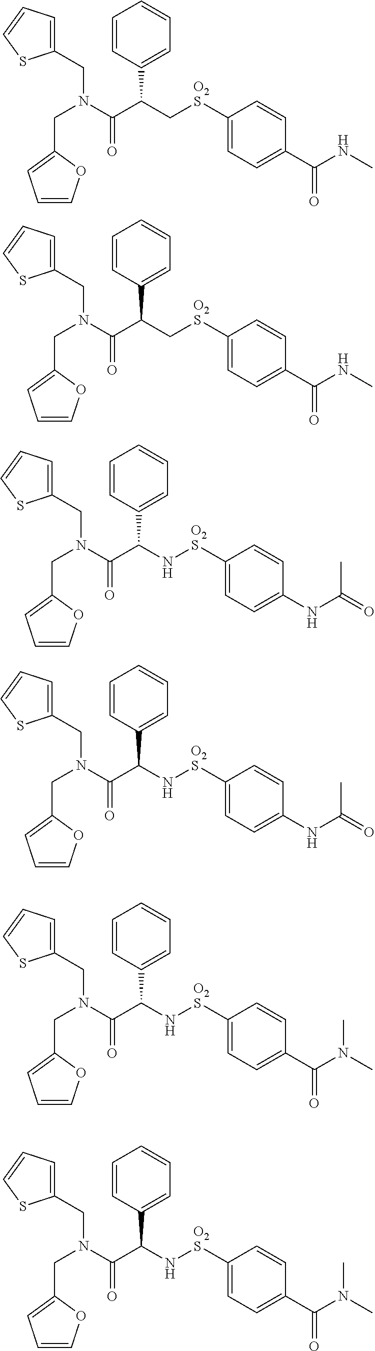

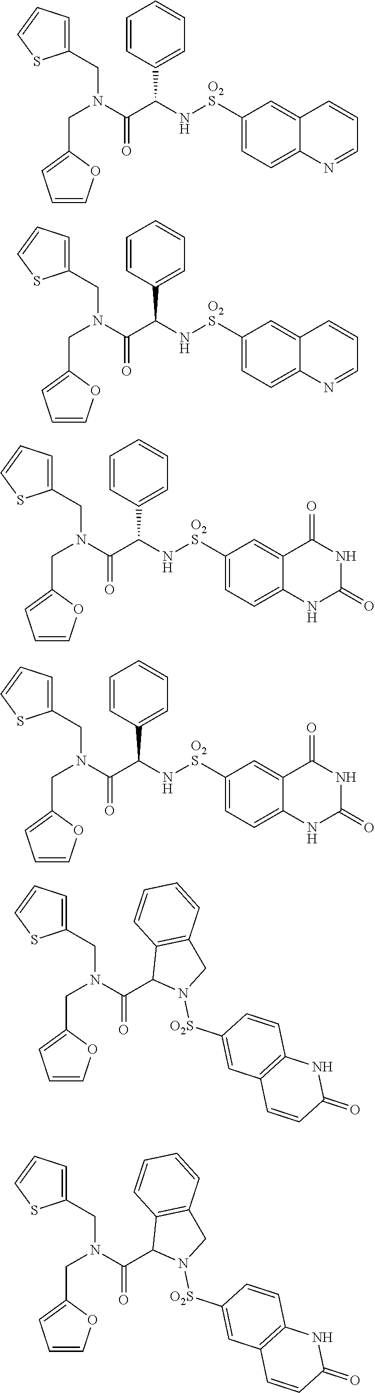

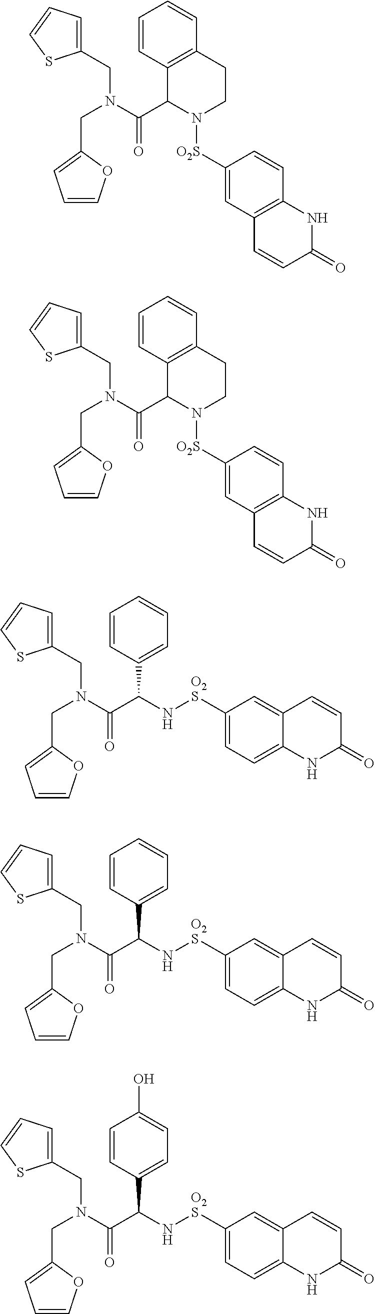 [SMILES: O=C(C1c2ccccc2CCN1S(=O)(=O)c1ccc2[nH]c(=O)ccc2c1)N(Cc1ccco1)Cc1cccs1 ; O=C(C1c2ccccc2CCN1S(=O)(=O)c1ccc2[nH]c(=O)ccc2c1)N(Cc1ccco1)Cc1cccs1 ; O=C([C@@H](NS(=O)(=O)c1ccc2[nH]c(=O)ccc2c1)c1ccccc1)N(Cc1ccco1)Cc1cccs1 ; O=C([C@H](NS(=O)(=O)c1ccc2[nH]c(=O)ccc2c1)c1ccccc1)N(Cc1ccco1)Cc1cccs1 ; O=C([C@H](NS(=O)(=O)c1ccc2[nH]c(=O)ccc2c1)c1ccc(O)cc1)N(Cc1ccco1)Cc1cccs1]

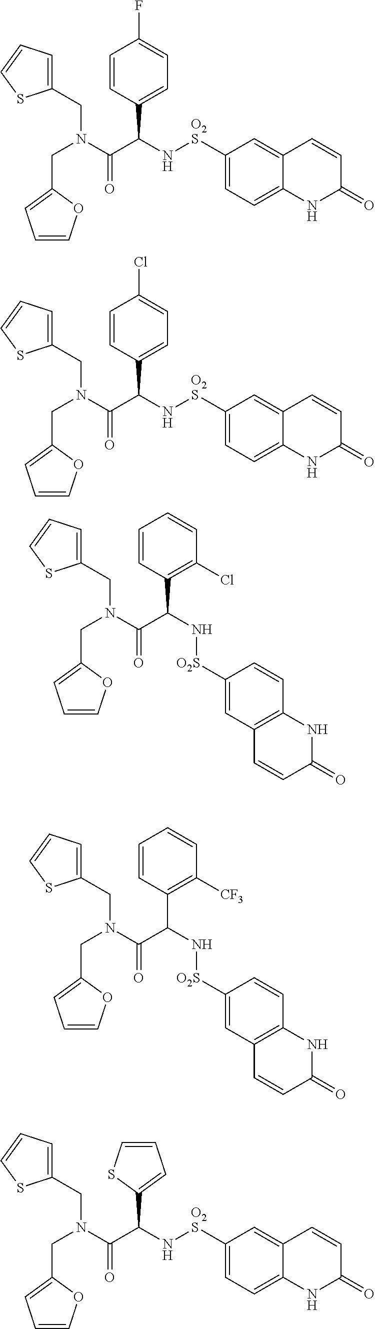

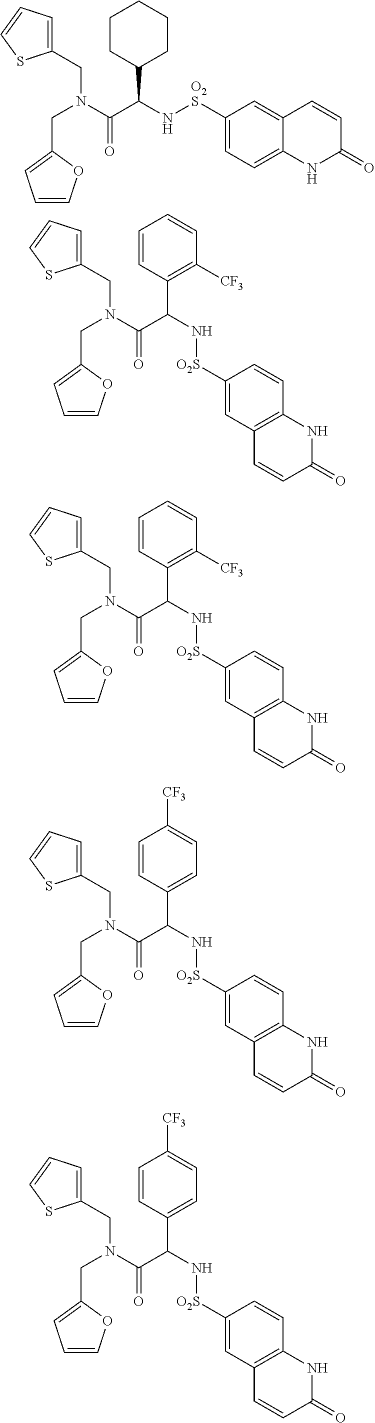

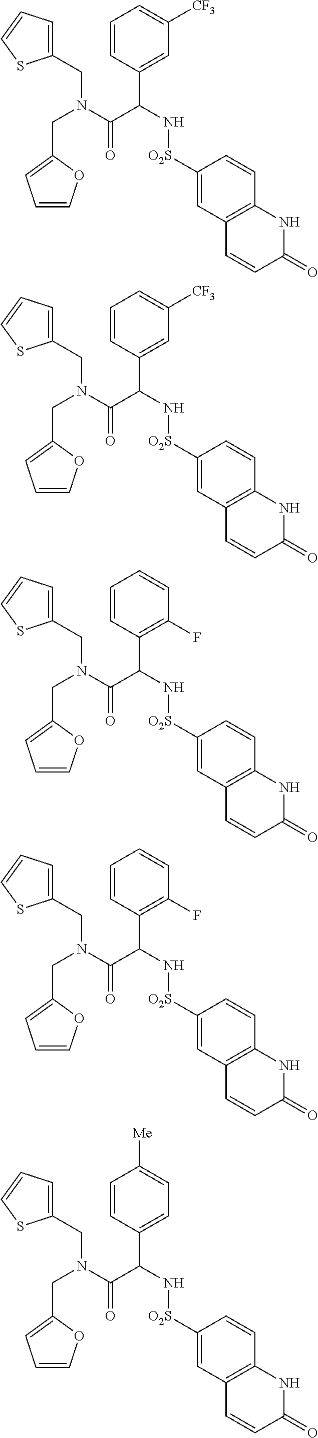

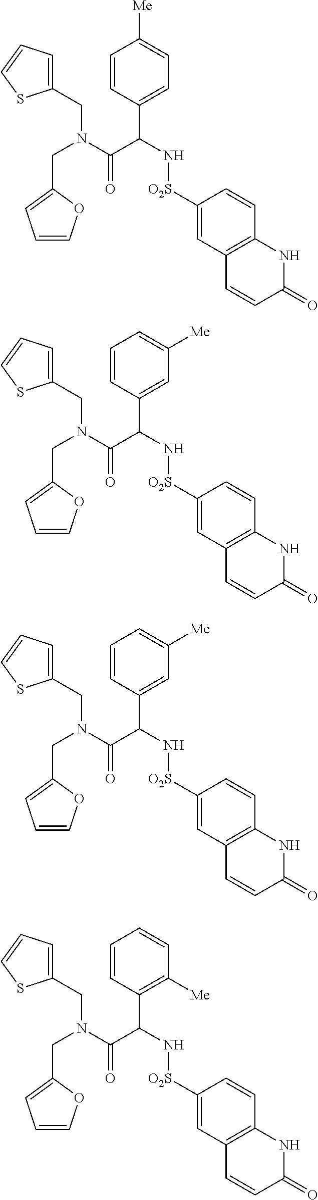 [SMILES: Cc1ccc(C(NS(=O)(=O)c2ccc3[nH]c(=O)ccc3c2)C(=O)N(Cc2ccco2)Cc2cccs2)cc1 ; Cc1cccc(C(NS(=O)(=O)c2ccc3[nH]c(=O)ccc3c2)C(=O)N(Cc2ccco2)Cc2cccs2)c1 ; Cc1cccc(C(NS(=O)(=O)c2ccc3[nH]c(=O)ccc3c2)C(=O)N(Cc2ccco2)Cc2cccs2)c1 ; Cc1ccccc1C(NS(=O)(=O)c1ccc2[nH]c(=O)ccc2c1)C(=O)N(Cc1ccco1)Cc1cccs1]

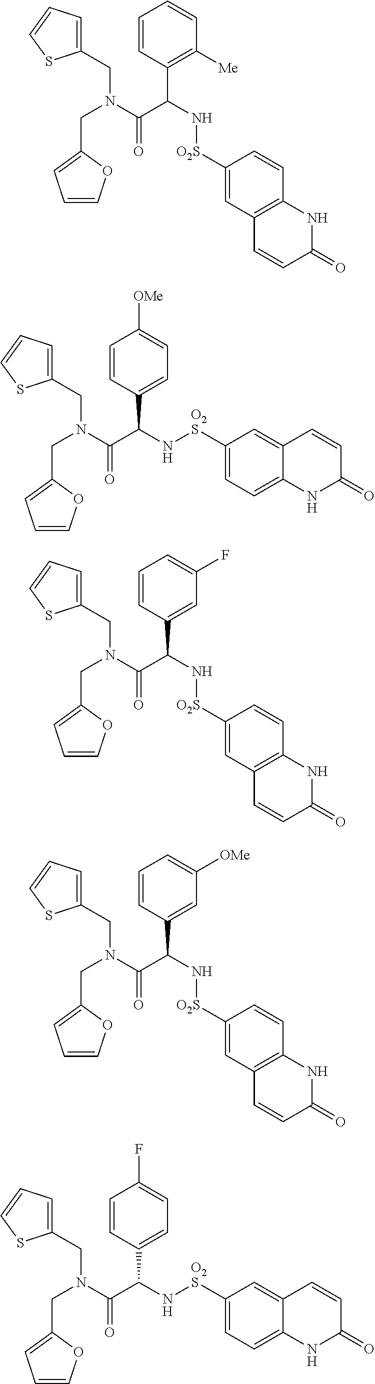

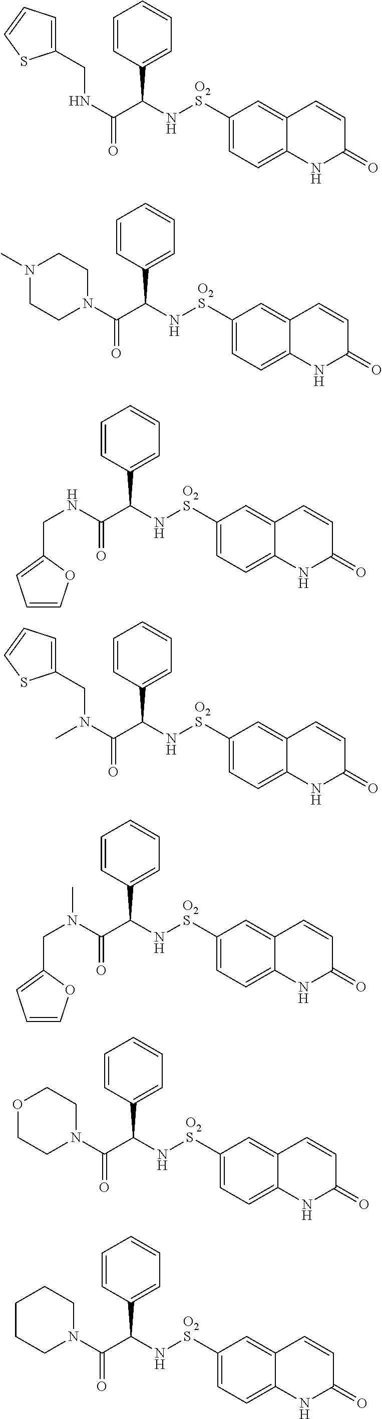

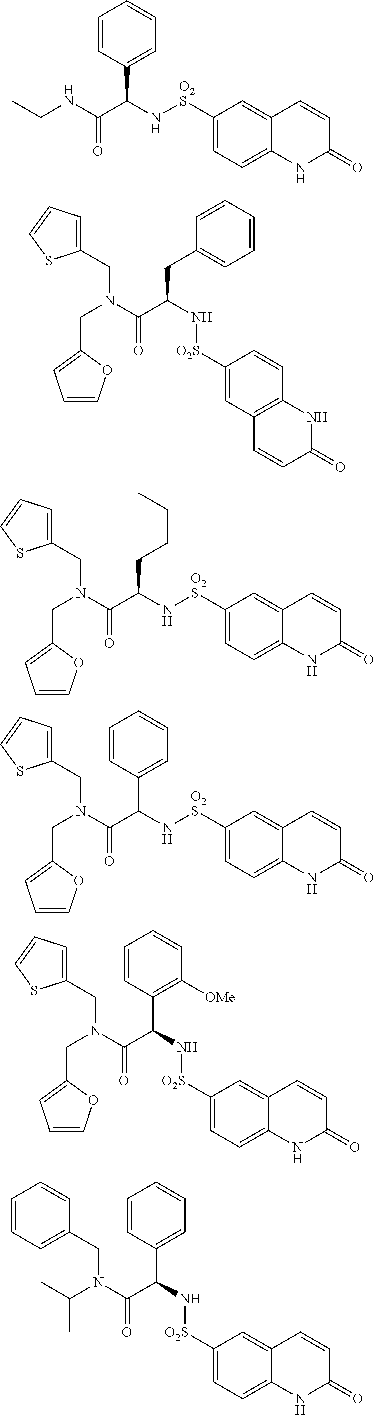

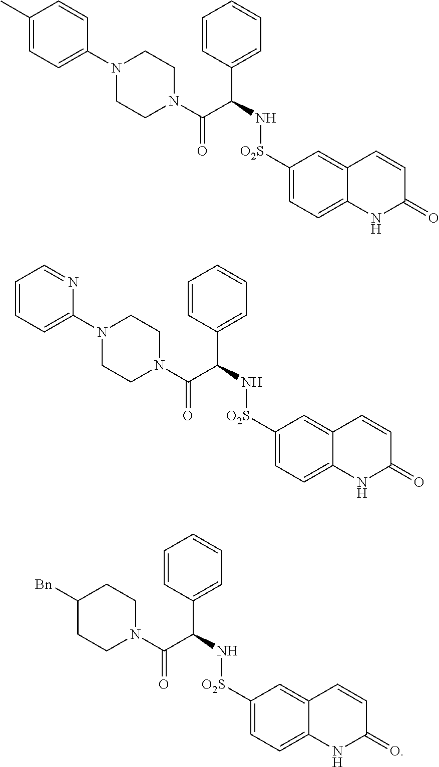 [SMILES: Cc1ccc(N2CCN(C(=O)[C@H](NS(=O)(=O)c3ccc4[nH]c(=O)ccc4c3)c3ccccc3)CC2)cc1 ; O=C([C@H](NS(=O)(=O)c1ccc2[nH]c(=O)ccc2c1)c1ccccc1)N1CCN(c2ccccn2)CC1 ; O=C([C@H](NS(=O)(=O)c1ccc2[nH]c(=O)ccc2c1)c1ccccc1)N1CCC(Cc2ccccc2)CC1]

46. The compound of claim 1, wherein the compound of formula (I) is not one of the following formulae:

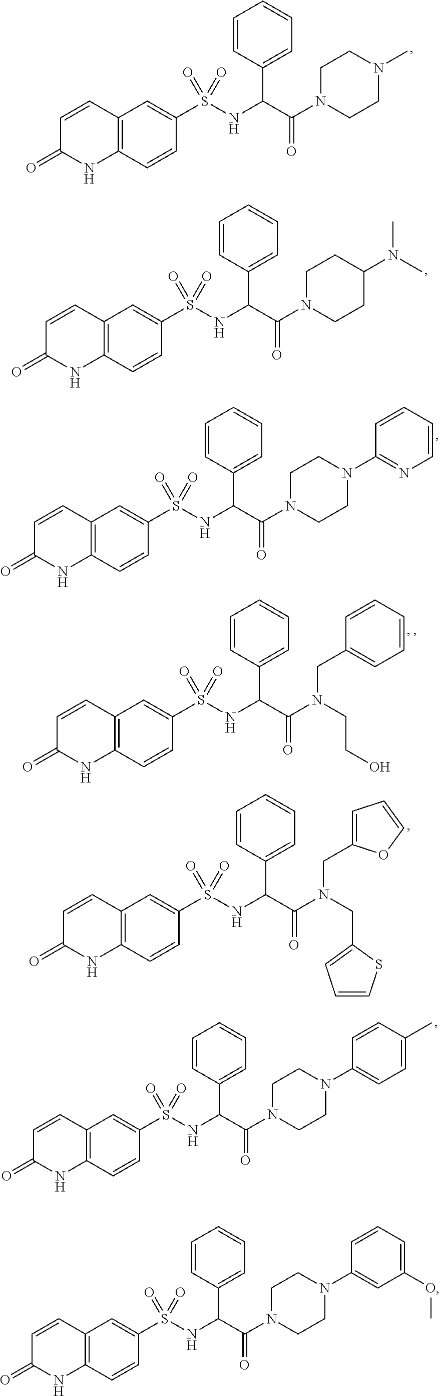

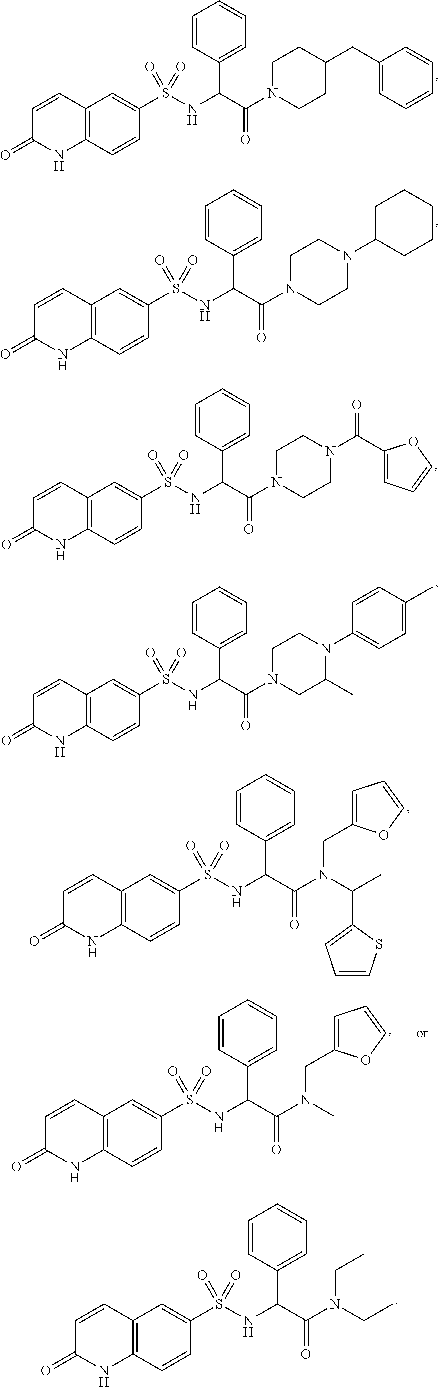

47. A pharmaceutical composition comprising a therapeutically effective amount of a compound of any one of claims 1-46; and a pharmaceutically acceptable excipient.

48. The composition of claim 47, administered in an amount sufficient to deliver about 0.001 mg/kg to about 100 mg/kg of subject body weight per day.

49. The composition of claim 47, administered in an amount sufficient to deliver about 0.01 mg/kg to about 10 mg/kg.

40. The composition of claim 47, administered in an amount sufficient to deliver about 0.1 mg/kg to about 40 mg/kg.

51. The composition of claim 47, administered in an amount sufficient to deliver about 1 mg/kg to about 25 mg/kg.

52. A method for treating an OGT-associated disease or condition in a subject comprising administering to a subject in need of such treatment a therapeutically effective amount of a compound of any one of claims 1-46 or a composition of claim 47.

53. The method of claim 52, wherein the subject is human.

54. The method of claim 52, wherein the compound or composition is administered in combination with an additional drug for treating an OGT-associated disease or disorder.

55. The method of claim 54, wherein the OGT-associated disease or condition is a neurodegenerative disease, cancer, metabolic disease, autoimmune disease, or inflammatory disease.

56. The method of claim 55, wherein the metabolic disease is diabetes mellitus type I, diabetes mellitus type II, insulin resistance, or a complication of diabetes.

57. The method of claim 56, wherein the complication of diabetes is insulin resistance, vascular disease, skin ulcers, circulatory damage, diabetic nephropathy, diabetic retinopathy, diabetic keratopathy, microvascular disease, macrovascular disease, or diabetic neuropathy.

58. The method of claim 55, wherein the neurodegenerative disease is Alzheimer's disease, progressive supranuclear palsy, corticobasal degeneration, frontotemporal lobar degeneration, or Pick's disease.

59. The method of claim 55, wherein the cancer is of the breast; biliary tract; bladder; bone; brain, including glioblastomas and medulloblastomas; central and peripheral nervous system; cervix; colon; connective tissue; endocrine glands (e.g., thyroid and adrenal cortex); esophagus; endometrium; germ cells; gastrointestinal tract; head and neck; kidney; liver; lung; larynx and hypopharynx; mesothelioma; muscle; ovary, including those arising from epithelial cells, stromal cells, germ cells and mesenchymal cells; pancreas; prostate; rectum; renal, including adenocarcinoma and Wilms tumor; small intestine; soft tissue; testis, including germinal tumors such as seminoma, non-seminoma (teratomas, choriocarcinomas), stromal tumors, and germ cell tumors; thyroid, including thyroid adenocarcinoma and medullar carcinoma; stomach; skin, including melanoma, Kaposi's sarcoma, basocellular cancer, and squamous cell cancer; ureter; vagina; and vulva; retinoblastoma; leukemia and lymphoma, namely non-Hodgkins disease, lymphocytic lymphomas, chronic and acute myeloid leukemia (CML/AML), acute lymphoblastic leukemia (ALL), chronic lymphocytic leukemia (CLL), Hodgkins disease, multiple myeloma, and T-cell lymphoma; myelodysplastic syndrome; plasma cell neoplasia; paraneoplastic syndromes; intraepithelial neoplasms including Bowen's disease and Paget's disease; neuroblastomas; oral cancer including squamous cell carcinoma; sarcomas including leiomyosarcoma, rhabdomyosarcoma, liposarcoma, fibrosarcoma, and osteosarcoma; cancers of unknown primary site; or AIDS-related malignancies.

60. The method of claim 55, wherein the autoimmune disease is inflammatory bowel disease, arthritis, systemic lupus erythematosus, rheumatoid arthritis, psoriatic arthritis, osteoarthritis, Still's disease, juvenile arthritis, diabetes, myasthenia gravis, Hashimoto's thyroiditis, Ord's thyroiditis, Graves' disease, Sjogren's syndrome, multiple sclerosis, Guillain-Barre syndrome, acute disseminated encephalomyelitis, Addison's disease, opsoclonus-myoclonus syndrome, ankylosing spondylosis, antiphospholipid antibody syndrome, aplastic anemia, autoimmune hepatitis, celiac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behcet's disease, chronic fatigue, dysautonomia, endometriosis, interstitial cystitis, neuromyotonia, scleroderma, or vulvodynia.

61. The method of claim 55, wherein the inflammatory disease is asthma, appendicitis, Blau syndrome, blepharitis, bronchiolitis, bronchitis, bursitis, cervicitis, cholangitis, cholecystitis, chronic obstructive pulmonary disease (COPD), chronic recurrent multifocal osteomyelitis (CRMO), colitis, conjunctivitis, cryopyrin associated periodic syndrome (CAPS), cystitis, dacryoadenitis, dermatitis, dermatomyositis, dry eye syndrome, encephalitis, endocarditis, endometritis, enteritis, enterocolitis, epicondylitis, epididymitis, familial cold-induced autoinflammatory syndrome, familial Mediterranean fever (FMF), fasciitis, fibrositis, gastritis, gastroenteritis, hepatitis, hidradenitis suppurativa, laryngitis, mastitis, meningitis, mevalonate kinase deficiency (MKD), Muckle-Well syndrome, myelitis myocarditis, myositis, nephritis, oophoritis, orchitis, osteitis, inflammatory osteolysis, otitis, pancreatitis, parotitis, pericarditis, peritonitis, pharyngitis, pleuritis, phlebitis, pneumonitis, pneumonia, proctitis, prostatitis, pulmonary fibrosis, pyelonephritis, pyoderma gangrenosum and acne syndrome (PAPA), pyogenic sterile arthritis, rhinitis, salpingitis, sinusitis, stomatitis, synovitis, systemic juvenile rheumatoid arthritis, tendonitis, TNF receptor associated periodic syndrome (TRAPS), tonsillitis, undifferentiated spondyloarthropathy, undifferentiated arthropathy, uveitis, vaginitis, vasculitis, vulvitis, or chronic inflammation resulting from chronic viral or bacteria infections, or psoriasis.

62. A method for inhibiting OGT activity in a cell comprising contacting a cell with an effective amount of a compound of any one of claims 1-46 to inhibit OGT.
